(19) United States
(12) Patent Application Publication
(10) Pub. No.: US 2026/0000757 A1
(43) Pub. Date: Jan. 1, 2026

(54) COMBINATION THERAPY FOR LUNG CANCER

(71) Applicant: Bristol-Myers Squibb Company, Princeton, NJ (US)

(72) Inventors: Mena Makram ABASKHAROUN, South Brunswick, NJ (US); Arun KUMAR, Cranbury, NJ (US)

(21) Appl. No.: 19/141,978

(22) PCT Filed: Dec. 20, 2023
(86) PCT No.: PCT/US2023/085070

§ 371 Date: Jun. 20, 2025

Related U.S. Application Data
(60) Provisional application No. 63/476,596, filed on Dec. 21, 2022.

(51) Int. Cl. A61K 39/395; A61K 31/282; A61K 31/337; A61K 31/4745; A61K 31/519; A61K 31/7048; A61K 31/7068; A61K 33/243; A61N 5/10; A61P 35/00; C07K 14/705; C07K 16/28

(52) U.S. Cl. CPC A61K 39/3955; A61K 31/282; A61K 31/337; A61K 31/4745; A61K 31/519; A61K 31/7048; A61K 31/7068; A61K 33/243; A61N 5/10; A61P 35/00; C07K 14/70532; C07K 16/2803; C07K 16/2818; C07K 16/2827; A61N 2005/1098; C07K 2317/21; C07K 2317/24; C07K 2317/31; C07K 2317/622; C07K 2319/31

(57) ABSTRACT

The disclosure provides a method of treating a human subject afflicted with lung cancer (e.g., non-small cell lung cancer (NSCLC)) with a programmed death-1 (PD-1) pathway inhibitor (e.g., an anti-PD-1 antibody) and a concurrent chemoradiotherapy (CCRT, e.g., a platinum doublet chemotherapy (PDCT) and a radiation therapy) followed by a combination of a PD-1 pathway inhibitor (e.g., an anti-PD-1 antibody) and a lymphocyte activation gene-3 (LAG-3) antagonist (e.g., an anti-LAG-3 antibody). In some aspects, the method comprises a recovery period that begins upon completion of the treatment with the PD-1 pathway inhibitor and the CCRT and ends at the start of the treatment with the combination of the PD-1 pathway inhibitor and the LAG-3 antagonist.

### CROSS REFERENCE TO RELATED APPLICATIONS

This PCT application claims the priority benefit of U.S. Provisional Application No. 63/476,596, filed Dec. 21, 2022, which is incorporated herein by reference in its entirety.

### REFERENCE TO SEQUENCE LISTING SUBMITTED ELECTRONICALLY

The content of the electronically submitted sequence listing (Name: 3338_323PC01_Seqlisting_ST26; Size: 102,064 bytes; and Date of Creation: Dec. 13, 2023) is herein incorporated by reference in its entirety.

### FIELD OF THE INVENTION

The present disclosure provides methods of treating human subjects afflicted with lung cancer comprising administering a programmed death-1 (PD-1) pathway inhibitor and a concurrent chemoradiotherapy followed by administering a PD-1 pathway inhibitor and a lymphocyte activation gene-3 (LAG-3) antagonist.

### BACKGROUND OF THE INVENTION

Lung cancer is the leading cause of cancer deaths worldwide (International Agency for Research on Cancer, GLOBOCAN Cancer Facts Sheet: Lung Cancer, 2020). An estimated 236,740 people will be diagnosed with lung cancer in 2022 in the US (American Cancer Society, Cancer Facts and Figures, 2022.) More than 350 people will die each day from lung cancer, which is more than breast, prostate, and pancreatic cancers combined, and 2.5 times more than colorectal carcinoma, the second leading cause of cancer death (Siegel et al., CA Cancer J. Clin. 2022; 72:7-33).

Non-small cell lung cancer (NSCLC) represents approximately 80% to 85% of all lung cancers and 30% of patients present with unresectable, locally advanced (Stage III) disease (GLOBOCAN Cancer Facts Sheet: Lung Cancer, 2020; Cancer Facts and Figures, 2022; Siegel et al.; Islami et al., CA Cancer J. Clin. 2018; 68:31-54). Unresectable, Stage III NSCLC represents heterogeneous disease requiring challenging, multi-modality treatment paradigms. Historically, the prognosis for this population has been suboptimal, with 5-year survival ranging from 10% to 30% (Curran et al., J. Natl. Cancer Inst. 2011; 103:1452-60; Bradley et al., Lancet Oncol. 2015; 16:187-99). Although considered the new standard-of-care, patient outcomes after definitive concurrent chemoradiotherapy followed by 12 months of durvalumab maintenance leaves a large fraction of patients with Stage III locally advanced NSCLC without appropriate treatment options (Spigel et al., J. Clin. Oncol. 2022; 40:1301-11).

There is a need for improved methods for treating human subjects afflicted with lung cancer.

### SUMMARY OF THE INVENTION

The present disclosure is directed to a method of treating a human subject afflicted with lung cancer, the method comprising administering to the subject: (a) a programmed death-1 (PD-1) pathway inhibitor and a concurrent chemoradiotherapy (CCRT), followed by (b) a PD-1 pathway inhibitor and a lymphocyte activation gene-3 (LAG-3) antagonist.

In some aspects, the method further comprises providing a recovery period to the subject that begins upon completion of the administration in (a) and ends upon the start of the administration in (b). In some aspects, the recovery period is a duration sufficient for the subject to recover from toxicities associated with the CCRT other than fatigue, esophagitis, or alopecia. In some aspects, the recovery period is from about 1 week to about 12 weeks, about 1 week to about 9 weeks, about 1 week to about 6 weeks, about 2 weeks to about 12 weeks, about 2 weeks to about 9 weeks, about 2 weeks to about 6 weeks, about 3 weeks to about 12 weeks, about 3 weeks to about 9 weeks, about 3 weeks to about 6 weeks, about 18 days from final administration of the PD-1 pathway inhibitor in (a) to about 12 weeks, about 18 days from final administration of the PD-1 pathway inhibitor in (a) to about 9 weeks, or about 18 days from final administration of the PD-1 pathway inhibitor in (a) to about 6 weeks.

In some aspects, the method is a first line therapy.

In some aspects, the subject has not received a prior local or systemic anticancer therapy given as primary therapy for locally advanced disease.

In some aspects, the subject has not received a prior systemic therapy for cancer, the subject has not received a prior systemic therapy for lung cancer, or the subject has not received a prior systemic therapy for advanced or metastatic lung cancer.

In some aspects, the subject is naïve to prior immuno-oncology therapy, the subject is naïve to prior immuno-oncology therapy for lung cancer, or the lung cancer is naïve to prior immuno-oncology therapy.

In some aspects, the method is a second line therapy. In some aspects, the method is a third line therapy. In some aspects, the subject has progressed on a prior therapy. In some aspects, the lung cancer is recurrent following multi-modal therapy for locally advanced lung cancer.

In some aspects, the lung cancer is unresectable, advanced, recurrent, and/or metastatic.

In some aspects, the lung cancer comprises small cell lung cancer.

In some aspects, the lung cancer comprises non-small cell lung cancer (NSCLC).

In some aspects, the NSCLC has a squamous or non-squamous histology. In some aspects, the NSCLC comprises locally advanced Stage IIIA, IIIB, or IIIC NSCLC.

In some aspects, the subject is free of progressive lung cancer during the CCRT or the recovery period.

In some aspects, the PD-1 pathway inhibitor in (a) and (b) are the same.

In some aspects, the PD-1 pathway inhibitor in (a) and (b) are different.

In some aspects, the PD-1 pathway inhibitor in (a) and/or (b) comprises an anti-PD-1 antibody and/or an anti-PD-L1 antibody.

In some aspects, the PD-1 pathway inhibitor in (a) and/or (b) comprises an anti-PD-1 antibody. In some aspects, the anti-PD-1 antibody comprises a full-length antibody. In some aspects, the anti-PD-1 antibody comprises a monoclonal, human, humanized, chimeric, or multispecific antibody. In some aspects, the multispecific antibody comprises a dual-affinity re-targeting antibody (DART), a DVD-Ig, or bispecific antibody. In some aspects, the anti-PD-1 antibody comprises a F(ab′)₂ fragment, a Fab′ fragment, a Fab fragment, a Fv fragment, a scFv fragment, a dsFv fragment, a dAb fragment, or a single chain binding polypeptide. In some aspects, the anti-PD-1 antibody comprises nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or an antigen binding portion thereof. In some aspects, the anti-PD-1 antibody comprises nivolumab or an antigen binding portion thereof. In some aspects, the anti-PD-1 antibody comprises CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14. In some aspects, the anti-PD-1 antibody comprises: (a) a heavy chain variable region CDR1 comprising the sequence set forth in SEQ ID NO: 15; (b) a heavy chain variable region CDR2 comprising the sequence set forth in SEQ ID NO: 16; (c) a heavy chain variable region CDR3 comprising the sequence set forth in SEQ ID NO:17; (d) a light chain variable region CDR1 comprising the sequence set forth in SEQ ID NO:18; (e) a light chain variable region CDR2 comprising the sequence set forth in SEQ ID NO:19; and (f) a light chain variable region CDR3 comprising the sequence set forth in SEQ ID NO:20. In some aspects, the anti-PD-1 antibody comprises heavy and light chain variable regions comprising the sequences set forth in SEQ ID NOs: 13 and 14, respectively. In some aspects, the anti-PD-1 antibody comprises heavy and light chains comprising the sequences as set forth in SEQ ID NOs: 11 and 12, respectively.

In some aspects, the PD-1 pathway inhibitor in (a) and/or (b) comprises a soluble PD-L2 polypeptide. In some aspects, the soluble PD-L2 polypeptide comprises a fusion polypeptide. In some aspects, the soluble PD-L2 polypeptide comprises a ligand binding fragment of the PD-L2 extracellular domain. In some aspects, the soluble PD-L2 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble PD-L2 polypeptide comprises AMP-224.

In some aspects, the PD-1 pathway inhibitor in (a) and/or (b) comprises an anti-PD-L1 antibody. In some aspects, the anti-PD-L1 antibody comprises a full-length antibody. In some aspects, the anti-PD-L1 antibody comprises a monoclonal, human, humanized, chimeric, or multispecific antibody. In some aspects, the multispecific antibody comprises a DART, a DVD-Ig, or bispecific antibody. In some aspects, the anti-PD-L1 antibody comprises a F(ab′)₂ fragment, a Fab′ fragment, a Fab fragment, a Fv fragment, a scFv fragment, a dsFv fragment, a dAb fragment, or a single chain binding polypeptide. In some aspects, the anti-PD-L1 antibody comprises BMS-936559, atezolizumab, durvalumab, avelumab, STI-1014, CX-072, KN035, LY3300054, BGB-A333, ICO 36, FAZ053, CK-301, or an antigen binding portion thereof.

In some aspects, the PD-1 pathway inhibitor in (a) and/or (b) comprises BMS-986189.

In some aspects, the PD-1 pathway inhibitor in (a) and/or (b) is administered at a flat dose. In some aspects, the PD-1 pathway inhibitor in (a) and/or (b) is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg. In some aspects, the PD-1 pathway inhibitor in (a) and/or (b) is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the PD-1 pathway inhibitor in (a) and/or (b) is administered as a weight-based dose. In some aspects, the PD-1 pathway inhibitor in (a) and/or (b) is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg. In some aspects, the PD-1 pathway inhibitor in (a) and/or (b) is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose of the PD-1 pathway inhibitor in (a) and (b) is different.

In some aspects, the dose of the PD-1 pathway inhibitor in (a) and/or (b) is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the CCRT comprises a platinum doublet chemotherapy (PDCT). In some aspects, the PDCT comprises a platinum agent in combination with a nucleoside analog, an antimetabolite, a taxane, a vinca alkaloid, or a topisomerase inhibitor. In some aspects, the platinum agent comprises cisplatin, carboplatin, oxaliplatin, satraplatin, picoplatin, nedaplatin, triplatin, lipoplatin, or phenanthriplatin. In some aspects, the platinum agent comprises cisplatin. In some aspects, the platinum agent comprises carboplatin. In some aspects, the nucleoside analog comprises cytarabine, gemcitabine, lamivudine, entecavir, or telbivudine. In some aspects, the nucleoside analog comprises gemcitabine. In some aspects, the antimetabolite comprises capecitabine, cladribine, clofarabine, cytarabine, floxuridine, fludarabine, fluorouracil, mercaptopurine, methotrexate, pemetrexed, pentostatin, pralatrexate, or thioguanine. In some aspects, the antimetabolite comprises pemetrexed. In some aspects, the taxane comprises paclitaxel, albumin-bound paclitaxel, docetaxel, or cabazitaxel. In some aspects, the vinca alkaloid comprises vinblastine, vincristine, vinorelbine, vindesine, vincaminol, vineridine, or vinburnine. In some aspects, the vinca alkaloid comprises vinorelbine or vinblastine. In some aspects, the topoisomerase inhibitor comprises etoposide, mitoxantrone, doxorubicin, irinotecan, topotecan, or camptothecin. In some aspects, the topoisomerase inhibitor comprises etoposide. In some aspects, the topoisomerase inhibitor comprises irinotecan. In some aspects, the PDCT comprises cisplatin or carboplatin in combination with gemcitabine, pemetrexed, paclitaxel, albumin-bound paclitaxel, docetaxel, vinorelbine, vinblastine, etoposide, or irinotecan. In some aspects, the PDCT comprises cisplatin or carboplatin in combination with paclitaxel or albumin-bound paclitaxel. In some aspects, the PDCT comprises cisplatin or carboplatin in combination with pemetrexed. In some aspects, the PDCT comprises cisplatin or carboplatin in combination with etoposide.

In some aspects, the CCRT comprises thoracic radiotherapy and/or volumetric-modulated arc therapy (VMAT), intensity-modulated radiation therapy (IMRT), or 3-dimensional conformal radiation therapy (3DRT).

In some aspects, the LAG-3 antagonist comprises an anti-LAG-3 antibody. In some aspects, the anti-LAG-3 antibody comprises a full-length antibody. In some aspects, the anti-LAG-3 antibody comprises a monoclonal, human, humanized, chimeric, or multispecific antibody. In some aspects, the multispecific antibody comprises a DART, a DVD-Ig, or bispecific antibody. In some aspects, the anti-LAG-3 antibody comprises a F(ab′)₂ fragment, a Fab′ fragment, a Fab fragment, a Fv fragment, a scFv fragment, a dsFv fragment, a dAb fragment, or a single chain binding polypeptide. In some aspects, the anti-LAG-3 antibody comprises BMS-986016 (relatlimab), IMP731 (H5L7BW), MK4280 (28G-10, favezelimab), REGN3767 (fianlimab), GSK2831781, humanized BAP050, IMP-701 (LAG525, ieramilimab), aLAG3 (0414), aLAG3 (0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGN02385, IBI-110, EMB-02, IBI-323, LBL-007, ABL501, or an antigen binding portion thereof. In some aspects, the anti-LAG-3 antibody comprises CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4. In some aspects, the anti-LAG-3 antibody comprises: (a) a heavy chain variable region CDR1 comprising the sequence set forth in SEQ ID NO:5; (b) a heavy chain variable region CDR2 comprising the sequence set forth in SEQ ID NO:6; (c) a heavy chain variable region CDR3 comprising the sequence set forth in SEQ ID NO:7; (d) a light chain variable region CDR1 comprising the sequence set forth in SEQ ID NO:8; (e) a light chain variable region CDR2 comprising the sequence set forth in SEQ ID NO:9; and (f) a light chain variable region CDR3 comprising the sequence set forth in SEQ ID NO: 10. In some aspects, the anti-LAG-3 antibody comprises heavy and light chain variable regions comprising the sequences set forth in SEQ ID NOs: 3 and 4, respectively. In some aspects, the anti-LAG-3 antibody comprises heavy and light chains comprising the sequences set forth in SEQ ID NOs: 1 and 2, respectively. In some aspects, the anti-LAG-3 antibody comprises heavy and light chains comprising the sequences set forth in SEQ ID NOs: 21 and 2, respectively.

In some aspects, the LAG-3 antagonist comprises a soluble LAG-3 polypeptide. In some aspects, the soluble LAG-3 polypeptide comprises a fusion polypeptide. In some aspects, the soluble LAG-3 polypeptide comprises a ligand binding fragment of the LAG-3 extracellular domain. In some aspects, the ligand binding fragment of the LAG-3 extracellular domain comprises an amino acid sequence with at least about 90%, at least about 95%, at least about 98%, at least about 99%, or about 100% sequence identity to SEQ ID NO: 22. In some aspects, the soluble LAG-3 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble LAG-3 polypeptide comprises IMP321 (eftilagimod alpha).

In some aspects, the LAG-3 antagonist is administered at a flat dose. In some aspects, the LAG-3 antagonist is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg. In some aspects, the LAG-3 antagonist is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the LAG-3 antagonist is administered at a weight-based dose. In some aspects, the LAG-3 antagonist is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg. In some aspects, the LAG-3 antagonist is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose of the LAG-3 antagonist is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the PD-1 pathway inhibitor in (a), the CCRT, the PD-1 pathway inhibitor in (b), and/or the LAG-3 antagonist is formulated for intravenous administration. In some aspects, the PD-1 pathway inhibitor in (b) and the LAG-3 antagonist are formulated separately. In some aspects, wherein the PD-1 pathway inhibitor in (b) is administered before the LAG-3 antagonist. In some aspects, the LAG-3 antagonist is administered before the PD-1 pathway inhibitor in (b). In some aspects, the PD-1 pathway inhibitor in (b) and the LAG-3 antagonist are administered concurrently.

In some aspects, the PD-1 pathway inhibitor in (b) and the LAG-3 antagonist are formulated together.

In some aspects, the PD-1 pathway inhibitor in (b) and the LAG-3 antagonist are administered as a maintenance therapy. In some aspects, the maintenance therapy is administered for up to about 1 year.

The present disclosure is directed to a method of treating a human subject afflicted with a NSCLC that has a squamous or non-squamous histology, the method comprising: (a) administering to the subject about 360 mg of an anti-PD-1 antibody comprising CDR1, CDR2 and CDR3 domains of the heavy chain variable region having the sequence set forth in SEQ ID NO: 13, and CDR1, CDR2 and CDR3 domains of the light chain variable region having the sequence set forth in SEQ ID NO:14, and a CCRT comprising a PDCT and radiotherapy, (b) providing the subject with a recovery period that begins upon completion of the administration in (a), followed by (c) administering to the subject a maintenance therapy comprising about 480 mg of an anti-PD-1 antibody comprising CDR1, CDR2 and CDR3 domains of the heavy chain variable region having the sequence set forth in SEQ ID NO: 13, and CDR1, CDR2 and CDR3 domains of the light chain variable region having the sequence set forth in SEQ ID NO:14, and about 480 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4.

In some aspects, the PDCT comprises cisplatin and etoposide. In some aspects, (a) comprises three 21-day cycles, wherein the anti-PD-1 antibody is administered on Day 1 of each cycle, wherein about 100 mg/m2 of the etoposide is administered on Days 1, 2, and 3 of each cycle, and after the anti-PD-1 antibody on Day 1 of each cycle, and wherein about 80 mg/m2 of the cisplatin is administered on Day 1 of each cycle after the etoposide. In some aspects, each of the cisplatin and the etoposide is administered intravenously over about 60 minutes.

In some aspects, the PDCT comprises carboplatin and paclitaxel. In some aspects, (a) comprises three 21-day cycles, wherein the anti-PD-1 antibody is administered on Day 1 of each cycle, wherein about 175 mg/m² or about 200 mg/m² of the paclitaxel is administered on Day 1 of the first cycle, wherein about 45 mg/m² or about 50 mg/m² of the paclitaxel is administered on Days 1, 8, and 15 of the second and third cycles, and wherein the paclitaxel is administered after the anti-PD-1 antibody on Day 1 of each cycle, and wherein the carboplatin at a target AUC of about 5 mg/mL·min or about 6 mg/mL·min is administered on Day 1 of the first cycle, wherein the carboplatin at a target AUC of about 2 mg/mL·min is administered on Days 1, 8, and 15 of the second and third cycles, and wherein the carboplatin is administered after the paclitaxel in each cycle. In some aspects, the carboplatin is administered intravenously over about 30 minutes, and the paclitaxel is administered intravenously over about 180 minutes in the first cycle and over about 60 minutes in the second and third cycles.

In some aspects, the PDCT comprises cisplatin and pemetrexed. In some aspects, (a) comprises three 21-day cycles, wherein the anti-PD-1 antibody is administered on Day 1 of each cycle, wherein about 500 mg/m² of the pemetrexed is administered on Day 1 of each cycle, after the anti-PD-1 antibody, and wherein about 75 mg/m² of the cisplatin is administered on Day 1 of each cycle after the pemetrexed. In some aspects, the cisplatin is administered intravenously over about 60 minutes and the pemetrexed is administered intravenously over about 10 minutes. In some aspects, the cisplatin is replaced with carboplatin at a target area under the concentration time-curve (AUC) of about 5 mg/mL·min if the cisplatin is not tolerated by the subject. In some aspects, the carboplatin is administered intravenously over about 30 minutes. In some aspects, the pemetrexed is replaced with etoposide if the pemetrexed is not tolerated by the subject.

In some aspects, the anti-PD-1 antibody in (a) is administered intravenously over about 30 minutes.

In some aspects, the radiotherapy is administered after the PDCT and comprises a dose of about 60 Gy to about 66 Gy. In some aspects, the radiotherapy comprises thoracic radiotherapy and/or volumetric-modulated arc therapy (VMAT), intensity-modulated radiation therapy (IMRT), or 3-dimensional conformal radiation therapy (3DRT). In some aspects, the radiotherapy begins on Day 1 of the second and third cycles and comprises about 30 to about 33 daily fractions of 2 Gy on a schedule of about 5 days on and 2 days off over about 6 weeks to about 7 weeks.

In some aspects, the recovery period is a duration sufficient for the subject to recover from toxicities associated with the CCRT other than fatigue, esophagitis, or alopecia. In some aspects, the recovery period is from about 1 week to about 12 weeks, about 1 week to about 9 weeks, about 1 week to about 6 weeks, about 2 weeks to about 12 weeks, about 2 weeks to about 9 weeks, about 2 weeks to about 6 weeks, about 3 weeks to about 12 weeks, about 3 weeks to about 9 weeks, about 3 weeks to about 6 weeks, about 18 days from final administration of the anti-PD-1 antibody in (a) to about 12 weeks, about 18 days from final administration of the anti-PD-1 antibody in (a) to about 9 weeks, or about 18 days from final administration of the anti-PD-1 antibody in (a) to about 6 weeks.

In some aspects, the method is a first line therapy.

In some aspects, the subject has not received a prior local or systemic anticancer therapy given as primary therapy for locally advanced disease.

In some aspects, the subject has not received a prior systemic therapy for cancer, the subject has not received a prior systemic therapy for lung cancer, or the subject has not received a prior systemic therapy for advanced or metastatic lung cancer.

In some aspects, the subject is naïve to prior immuno-oncology therapy, the subject is naïve to prior immuno-oncology therapy for lung cancer, or the lung cancer is naïve to prior immuno-oncology therapy.

In some aspects, the method is a second line therapy. In some aspects, the method is a third line therapy. In some aspects, the subject has progressed on a prior therapy. In some aspects, the NSCLC is recurrent following multi-modal therapy for locally advanced NSCLC.

In some aspects, the NSCLC is unresectable, advanced, recurrent, and/or metastatic.

In some aspects, the NSCLC has a squamous or non-squamous histology.

In some aspects, the NSCLC comprises locally advanced Stage IIIA, IIIB, or IIIC NSCLC.

In some aspects, the subject is free of progressive NSCLC during the CCRT or the recovery period.

In some aspects, the anti-PD-1 and the anti-LAG-3 antibodies in (b) are administered once about every 4 weeks.

In some aspects, the anti-PD-1 and the anti-LAG-3 antibodies in (b) are formulated separately. In some aspects, the anti-PD-1 antibody in (b) is administered before the anti-LAG-3 antibody. In some aspects, the anti-LAG-3 antibody is administered before the anti-PD-1 antibody in (b). In some aspects, the anti-PD-1 and the anti-LAG-3 antibodies in (b) are administered concurrently.

In some aspects, the anti-PD-1 and the anti-LAG-3 antibodies in (b) are formulated together.

In some aspects, the anti-PD-1 and the anti-LAG-3 antibodies in (b) are administered intravenously over about 30 minutes.

In some aspects, the anti-PD-1 antibody in (a) and/or (b) comprises a full-length antibody. In some aspects, the anti-PD-1 antibody in (a) and/or (b) comprises a monoclonal, human, humanized, chimeric, or multispecific antibody. In some aspects, the multispecific antibody comprises a DART, a DVD-Ig, or bispecific antibody. In some aspects, the anti-PD-1 antibody in (a) and/or (b) comprises a F(ab′)₂ fragment, a Fab′ fragment, a Fab fragment, a Fv fragment, a scFv fragment, a dsFv fragment, a dAb fragment, or a single chain binding polypeptide. In some aspects, the anti-PD-1 antibody in (a) and/or (b) comprises: (a) a heavy chain variable region CDR1 comprising the sequence set forth in SEQ ID NO:15; (b) a heavy chain variable region CDR2 comprising the sequence set forth in SEQ ID NO:16; (c) a heavy chain variable region CDR3 comprising the sequence set forth in SEQ ID NO:17; (d) a light chain variable region CDR1 comprising the sequence set forth in SEQ ID NO:18; (e) a light chain variable region CDR2 comprising the sequence set forth in SEQ ID NO:19; and (f) a light chain variable region CDR3 comprising the sequence set forth in SEQ ID NO:20. In some aspects, the anti-PD-1 antibody in (a) and/or (b) comprises heavy and light chain variable regions comprising the sequences set forth in SEQ ID NOs: 13 and 14, respectively. In some aspects, the anti-PD-1 antibody in (a) and/or (b) comprises heavy and light chains comprising the sequences as set forth in SEQ ID NOs: 11 and 12, respectively.

In some aspects, the anti-LAG-3 antibody comprises a full-length antibody. In some aspects, the anti-LAG-3 antibody comprises a monoclonal, human, humanized, chimeric, or multispecific antibody. In some aspects, the multispecific antibody comprises a DART, a DVD-Ig, or bispecific antibody. In some aspects, the anti-LAG-3 antibody comprises a F(ab′)₂ fragment, a Fab′ fragment, a Fab fragment, a Fv fragment, a scFv fragment, a dsFv fragment, a dAb fragment, or a single chain binding polypeptide. In some aspects, the anti-LAG-3 antibody comprises: (a) a heavy chain variable region CDR1 comprising the sequence set forth in SEQ ID NO:5; (b) a heavy chain variable region CDR2 comprising the sequence set forth in SEQ ID NO:6; (c) a heavy chain variable region CDR3 comprising the sequence set forth in SEQ ID NO:7; (d) a light chain variable region CDR1 comprising the sequence set forth in SEQ ID NO:8; (e) a light chain variable region CDR2 comprising the sequence set forth in SEQ ID NO:9; and (f) a light chain variable region CDR3 comprising the sequence set forth in SEQ ID NO:10. In some aspects, the anti-LAG-3 antibody comprises heavy and light chain variable regions comprising the sequences set forth in SEQ ID NOs: 3 and 4, respectively. In some aspects, the anti-LAG-3 antibody comprises heavy and light chains comprising the sequences set forth in SEQ ID NOs: 1 and 2, respectively. In some aspects, the anti-LAG-3 antibody comprises heavy and light chains comprising the sequences set forth in SEQ ID NOs: 21 and 2, respectively.

In some aspects, one or more immune cells in tumor tissue from the subject express LAG-3. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3. In some aspects, at least about 1% of the immune cells express LAG-3. In some aspects, the immune cells comprise tumor-infiltrating lymphocytes. In some aspects, the tumor-infiltrating lymphocytes comprise CD8⁺ cells.

In some aspects, one or more nucleated cells in tumor tissue from the subject express LAG-3. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the nucleated cells express LAG-3. In some aspects, at least about 1% of the nucleated cells express LAG-3.

In some aspects, one or more tumor cells in tumor tissue from the subject express PD-L1. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1. In some aspects, at least about 1% of the tumor cells express PD-L1.

In some aspects, any of the methods further comprise administering to the subject an additional therapeutic agent. In some aspects, the additional therapeutic agent comprises an anti-cancer agent. In some aspects, the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof. In some aspects, the tyrosine kinase inhibitor comprises afatinib, erlotinib, dacomitinib, gefitinib, osimertinib, alectinib, brigatinib, ceritinib, crizotinib, lorlatinib, entrectinib, dabrafenib, trametinib, vemurafenib, larotrectinib, or any combination thereof. In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGF receptor (EGFR), or any combination thereof. In some aspects, the anti-angiogenesis agent comprises bevacizumab, ramucirumab, aflibercept, tanibirumab, olaratumab, nesvacumab, AMG780, MEDI3617, vanucizumab, rilotumumab, ficlatuzumab, TAK-701, onartuzumab, emibetuzumab, or any combination thereof. In some aspects, the checkpoint inhibitor comprises a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM⁻¹ inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM⁻¹) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof. In some aspects, the checkpoint inhibitor comprises a CTLA-4 inhibitor. In some aspects, the CTLA-4 inhibitor comprises an anti-CTLA-4 antibody. In some aspects, the anti-CTLA-4 antibody comprises a full-length antibody. In some aspects, the anti-CTLA-4 antibody comprises a monoclonal, human, humanized, chimeric, or multispecific antibody. In some aspects, the multispecific antibody comprises a DART, a DVD-Ig, or bispecific antibody. In some aspects, the anti-CTLA-4 antibody comprises a F(ab′)₂ fragment, a Fab′ fragment, a Fab fragment, a Fv fragment, a scFv fragment, a dsFv fragment, a dAb fragment, or a single chain binding polypeptide. In some aspects, the anti-CTLA-4 antibody comprises ipilimumab, tremelimumab, MK-1308, AGEN-1884, or an antigen binding portion thereof.

### DETAILED DESCRIPTION OF THE INVENTION

The present disclosure provides a method of treating a human subject afflicted with lung cancer (e.g., non-small cell lung cancer (NSCLC)), the method comprising administering to the subject: (a) a programmed death-1 (PD-1) pathway inhibitor (e.g., an anti-PD-1 antibody) and a concurrent chemoradiotherapy (CCRT, e.g., a platinum doublet chemotherapy (PDCT) and a radiation therapy) followed by (b) a PD-1 pathway inhibitor (e.g., an anti-PD-1 antibody) and a lymphocyte activation gene-3 (LAG-3) antagonist (e.g., an anti-LAG-3 antibody). In some aspects, the method comprises a recovery period (e.g., of about 3 weeks to about 6 weeks) that begins upon completion of the administering of the PD-1 pathway inhibitor and the CCRT in (a) and ends upon the start of the administering of the PD-1 pathway inhibitor and the LAG-3 antagonist in (b). In some aspects, the PD-1 pathway inhibitor and the LAG-3 antagonist in (b) are administered as a maintenance therapy (e.g., for up to one year).

### I. Terms

In order that the present disclosure can be more readily understood, certain terms are first defined. As used in this application, except as otherwise expressly provided herein, each of the following terms shall have the meaning set forth below. Additional definitions are set forth throughout the application. It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

The headings provided herein are not limitations of the various aspects of the disclosure, which can be had by reference to the specification as a whole. Accordingly, the terms defined immediately below are more fully defined by reference to the specification in its entirety.

An “antagonist” shall include, without limitation, any molecule capable of blocking, reducing, or otherwise limiting an interaction or activity of a target molecule (e.g., LAG-3). In some aspects, the antagonist is an antibody. In other aspects, the antagonist comprises a small molecule. The terms “antagonist” and “inhibitor” are used interchangeably herein.

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as V_{H}) and a heavy chain constant region (abbreviated herein as C_{H}). The heavy chain constant region comprises three constant domains, C_{H1}, C_{H2} and C_{H3}. Each light chain comprises a light chain variable region (abbreviated herein as V_{L}) and a light chain constant region (abbreviated herein as C_{L}). The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. A heavy chain can have the C-terminal lysine or not. Unless specified otherwise herein, the amino acids in the variable regions are numbered using the Kabat numbering system and those in the constant regions are numbered using the EU system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; single chain antibodies; monospecific antibodies; bispecific antibodies; and multi-specific antibodies. A nonhuman antibody can be humanized by recombinant methods to reduce its immunogenicity in humans. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, that retains the ability to bind specifically to the antigen bound by the whole immunoglobulin. Examples of an “antigen-binding portion” or “antigen-binding fragment” include: (1) a Fab fragment (fragment from papain cleavage) or a similar monovalent fragment consisting of the V_{L}, V_{H}, Le and CHI domains; (2) a F(ab′)2 fragment (fragment from pepsin cleavage) or a similar bivalent fragment comprising two Fab fragments linked by a disulfide bridge at the hinge region; (3) a Fd fragment consisting of the V_{H} and CHI domains; (4) a Fv fragment consisting of the V_{L} and V_{H} domains of a single arm; (5) a single domain antibody (dAb) fragment (Ward et al., (1989) Nature 341:544-46), which consists of a V_{H} domain; (6) a bi-single domain antibody which consists of two V_{H} domains linked by a hinge (dual-affinity re-targeting antibodies (DARTs)); or (7) a dual variable domain immunoglobulin. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883).

An “isolated antibody” refers to an antibody that is substantially free of other antibodies having different antigenic specificities (e.g., an isolated antibody that binds specifically to LAG-3 is substantially free of antibodies that do not bind specifically to LAG-3). An isolated antibody that binds specifically to LAG-3 can, however, have cross-reactivity to other antigens, such as LAG-3 molecules from different species. Moreover, an isolated antibody can be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs can be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-LAG-3 antibody binds specifically to LAG-3.

“LAG-3” refers to Lymphocyte Activation Gene-3. The term “LAG-3” includes variants, isoforms, homologs, orthologs and paralogs. For example, antibodies specific for a human LAG-3 protein can, in certain cases, cross-react with a LAG-3 protein from a species other than human. In other aspects, the antibodies specific for a human LAG-3 protein can be completely specific for the human LAG-3 protein and not exhibit species or other types of cross-reactivity, or can cross-react with LAG-3 from certain other species, but not all other species (e.g., cross-react with monkey LAG-3 but not mouse LAG-3). The term “human LAG-3” refers to human sequence LAG-3, such as the complete amino acid sequence of human LAG-3 having GenBank Accession No. NP_002277 The term “mouse LAG-3” refers to mouse sequence LAG-3, such as the complete amino acid sequence of mouse LAG-3 having GenBank Accession No. NP_032505. LAG-3 is also known in the art as, for example, CD223. The human LAG-3 sequence can differ from human LAG-3 of GenBank Accession No. NP_002277 by having, e.g., conserved mutations or mutations in non-conserved regions, and the LAG-3 has substantially the same biological function as the human LAG-3 of GenBank Accession No. NP_002277. For example, a biological function of human LAG-3 is having an epitope in the extracellular domain of LAG-3 that is specifically bound by an antibody of the instant disclosure or a biological function of human LAG-3 is binding to MHC Class II molecules.

A particular human LAG-3 sequence will generally be at least about 90% identical in amino acid sequence to human LAG-3 of GenBank Accession No. NP_002277 and contains amino acid residues that identify the amino acid sequence as being human when compared to LAG-3 amino acid sequences of other species (e.g., murine). In certain cases, a human LAG-3 can be at least about 95%, or even at least about 96%, at least about 97%, at least about 98%, at least about 99%, or about 100% identical in amino acid sequence to LAG-3 of GenBank Accession No. NP_002277. In certain aspects, a human LAG-3 sequence will display no more than 10 amino acid differences from the LAG-3 sequence of GenBank Accession No. NP_002277. In certain aspects, the human LAG-3 can display no more than 5, or even no more than 4, 3, 2, or 1 amino acid difference from the LAG-3 sequence of GenBank Accession No. NP_002277.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863. “PD-1” and “PD-1 receptor” are used interchangeably herein.

“Cytotoxic T-Lymphocyte Antigen-4 (CTLA-4)” refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

“Programmed Death Ligand-2 (PD-L2)” as used herein includes human PD-L2 (hPD-L2), variants, isoforms, and species homologs of hPD-L2, and analogs having at least one common epitope with hPD-L2. The complete hPD-L2 sequence can be found under GenBank Accession No. Q9BQ51.

A “patient” as used herein includes any patient who is afflicted with a lung cancer (e.g., NSCLC). The terms “subject” and “patient” are used interchangeably herein.

“Administering” refers to the physical introduction of a therapeutic agent to a subject (e.g., a composition or formulation comprising the therapeutic agent), using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, in some aspects, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease. Response Evaluation Criteria In Solid Tumors (RECIST) is a measure for treatment efficacy and are established rules that define when tumors respond, stabilize, or progress during treatment. RECIST 1.1 is the current guideline to solid tumor measurement and definitions for objective assessment of change in tumor size for use in adult and pediatric cancer clinical trials.

As used herein, “maintenance therapy” refers to a therapy that is intended to prevent the occurrence or recurrence of tumors.

As used herein, “a recovery period” is a duration beginning upon the completion of one therapy and ending upon the start of another therapy (e.g., a duration that begins upon the completion of a CCRT as disclosed herein and that ends upon the administration of a combination therapy and/or maintenance therapy as disclosed herein, such as the combination of a PD-1 pathway inhibitor and a LAG-3 antagonist as disclosed herein). In some aspects, the recovery period is a duration sufficient for a subject to recover from adverse events or serious adverse events associated with a therapy (e.g., a duration sufficient for the subject to recover from toxicities associated with a CCRT as disclosed herein other than fatigue, esophagitis, or alopecia).

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of solid tumor. Effective treatment can refer to alleviation of at least one symptom of a solid tumor. Such effective treatment can, e.g., reduce patient pain, reduce the size and/or number of lesions, can reduce or prevent metastasis of a tumor, and/or can slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to delay other unwanted cell proliferation. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of anti-LAG-3 antibody alone or the amount of anti-LAG-3 antibody and the amount an additional therapeutic agent (e.g., anti-PD-1 antibody), in combination, clinically proven to affect a significant decrease in cancer or slowing of progression of cancer, such as an advanced solid tumor.

As used herein, the terms “fixed dose,” “flat dose,” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., an amount in μg or mg).

The use of the term “fixed dose combination” with regard to a composition of the invention means that two or more different inhibitors as described herein (e.g., an anti-LAG-3 antibody and an anti-PD-1 antibody) in a single composition are present in the composition in particular (fixed) ratios with each other. In some aspects, the fixed dose is based on the weight (e.g., mg) of the inhibitors. In certain aspects, the fixed dose is based on the concentration (e.g., mg/ml) of the inhibitors. In some aspects, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first inhibitor to mg second inhibitor. For example, a 1:1 ratio of a first inhibitor and a second inhibitor can mean that a vial can contain about 480 mg of the first inhibitor and 480 mg of the second inhibitor or about 12 mg/ml of the first inhibitor and 12 mg/ml of the second inhibitor.

The term “weight based dose” as referred to herein means that a dose that is administered to a patient is calculated based on the weight of the patient.

“Dosing interval,” as used herein, means the amount of time that elapses between multiple doses of a formulation disclosed herein being administered to a subject. Dosing interval can thus be indicated as ranges.

The term “dosing frequency” as used herein refers to the frequency of administering doses of a formulation disclosed herein in a given time. Dosing frequency can be indicated as the number of doses per a given time, e.g., once a week or once in two weeks, etc.

The terms “about once a week,” “once about every week,” “once about every two weeks,” or any other similar dosing interval terms as used herein means approximate number, and “about once a week” or “once about every week” can include every seven days #two days, i.e., every five days to every nine days. The dosing frequency of “once a week” thus can be every five days, every six days, every seven days, every eight days, or every nine days. “Once about every three weeks” can include every 21 days+3 days, i.e., every 25 days to every 31 days. Similar approximations apply, for example, to once about every two weeks, once about every four weeks, once about every five weeks, once about every six weeks, once about every seven weeks, once about every eight weeks, once about every nine weeks, once about every ten weeks, once about every eleven weeks, and once about every twelve weeks. In some aspects, a dosing interval of once about every six weeks or once about every twelve weeks means that the first dose can be administered any day in the first week, and then the next dose can be administered any day in the sixth or twelfth week, respectively. In other aspects, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity.

The term “tumor” as used herein refers to any mass of tissue that results from excessive cell growth or proliferation, either benign (non-cancerous) or malignant (cancerous), including pre-cancerous lesions.

The term “biological sample” as used herein refers to biological material isolated from a subject. The biological sample can contain any biological material suitable for analysis, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. The biological sample can be a test tissue sample (e.g., a tissue sample comprising tumor cells and tumor-infiltrating inflammatory cells). In one aspect, the sample is a tumor tissue biopsy, e.g., a formalin-fixed, paraffin-embedded (FFPE) tumor tissue or a fresh-frozen tumor tissue or the like. In another aspect, the biological sample is a liquid biopsy that, in some aspects, comprises one or more of blood, serum, plasma, circulating tumor cells, exoRNA, ctDNA, and cfDNA.

By way of example, an “anti-cancer agent” promotes cancer regression in a subject. In preferred aspects, a therapeutically effective amount of the agent promotes cancer regression to the point of eliminating the cancer. “Promoting cancer regression” means that administering an effective amount of the anti-cancer agent, alone or in combination with another agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the agent to promote cancer regression in the patient. Physiological safety refers to the level of toxicity, or other adverse physiological effects at the cellular, organ and/or organism level (adverse effects) resulting from administration of the agent.

By way of example for the treatment of tumors, a therapeutically effective amount of an anti-cancer agent can inhibit cell growth or tumor growth by at least about 20%, at least about 40%, at least about 60%, or at least about 80% relative to untreated subjects. In other aspects of the disclosure, tumor regression can be observed and continue for a period of at least about 20 days, more preferably at least about 40 days, or at least about 60 days. Notwithstanding these measurements of therapeutic effectiveness, evaluation of immunotherapeutic drugs must also make allowance for immune-related response patterns.

As used herein, an “immuno-oncology” therapy or an “I-O” or “IO” therapy refers to a therapy that comprises utilizing an immune response to target and treat a tumor in a subject. As such, as used herein, an I-O therapy is a type of anti-cancer therapy. In some aspects, an I-O therapy comprises administering an antibody to a subject. In some aspects, an I-O therapy comprises administering to a subject an immune cell, e.g., a T cell, e.g., a modified T cell, e.g., a T cell modified to express a chimeric antigen receptor or a particular T cell receptor. In some aspects, the I-O therapy comprises administering a therapeutic vaccine to a subject. In some aspects, the I-O therapy comprises administering a cytokine or a chemokine to a subject. In some aspects, the I-O therapy comprises administering an interleukin to a subject. In some aspects, the I-O therapy comprises administering an interferon to a subject. In some aspects, the I-O therapy comprises administering a colony stimulating factor to a subject.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

A “tumor-infiltrating inflammatory cell” or “tumor-associated inflammatory cell” is any type of cell that typically participates in an inflammatory response in a subject and which infiltrates tumor tissue. Such cells include tumor-infiltrating lymphocytes (TILs), macrophages, monocytes, eosinophils, histiocytes and dendritic cells.

The term “LAG-3 positive” or “LAG-3 expression positive,” relating to LAG-3 expression, refers to tumor tissue (e.g., a test tissue sample) that is scored as expressing LAG-3 based on the proportion (i.e., percentage) of immune cells (e.g., tumor-infiltrating lymphocytes such as CD8+ T cells) expressing LAG-3 (e.g., greater than or equal to 1% expression) or the proportion (i.e., percentage) of nucleated cells expressing LAG-3 (i.e., the immune cells that express LAG-3 as a proportion of total nucleated cells, e.g., greater than or equal to 1% expression).

“LAG-3 negative” or “LAG-3 expression negative,” refers to tumor tissue (e.g., a test tissue sample) that is not scored as expressing LAG-3 (e.g., less than 1% LAG-3 expression in immune cells and/or nucleated cells).

The term “PD-1 positive” or “PD-1 expression positive,” relating to PD-1 expression, refers to tumor tissue (e.g., a test tissue sample) that is scored as expressing PD-1 based on the proportion (i.e., percentage) of immune cells (e.g., tumor-infiltrating lymphocytes such as CD8+ T cells) expressing PD-1 (e.g., greater than or equal to 1% expression) or the proportion (i.e., percentage) of nucleated cells expressing PD-1 (i.e., the immune cells that express PD-1 as a proportion of total nucleated cells, e.g., greater than or equal to 1% expression).

“PD-1 negative” or “PD-1 expression negative,” refers to tumor tissue (e.g., a test tissue sample) that is not scored as expressing PD-1 (e.g., less than 1% PD-1 expression).

The term “PD-L1 positive” or “PD-L1 expression positive,” relating to cell surface PD-L1 expression, refers to tumor tissue (e.g., a test tissue sample) that is scored as expressing PD-L1 based on the proportion (i.e., percentage) of tumor cells expressing PD-L1 (e.g., greater than or equal to 1% expression) or the proportion (i.e., percentage) of nucleated cells expressing PD-L1 (i.e., the tumor cells that express PD-L1 as a proportion of total nucleated cells, e.g., greater than or equal to 1% expression).

The term “PD-L1 negative” or “PD-L1 expression negative” refers to tumor tissue (e.g., a test tissue sample) that is not scored as expressing PD-L1 (e.g., less than 1% expression).

Various aspects of the invention are described in further detail in the following subsections.

### II. Methods of the Disclosure

Provided herein are methods of treating a human subject afflicted with lung cancer, the methods comprising administering to the subject: (a) a programmed death-1 (PD-1) pathway inhibitor (e.g., an anti-PD-1 antibody) and a concurrent chemoradiotherapy (CCRT, i.e., concurrent chemotherapy and radiotherapy, e.g., a platinum double chemotherapy (PDCT) and a radiotherapy), followed by (b) a PD-1 pathway inhibitor (e.g., an anti-PD-1 antibody) and a lymphocyte activation gene-3 (LAG-3) antagonist (e.g., an anti-LAG-3 antibody). The administration in (b) is also interchangeably referred to herein as a combination therapy comprising a PD-1 pathway inhibitor and a LAG-3 antagonist (e.g., a fixed dose combination comprising a PD-1 pathway inhibitor and a LAG-3 antagonist).

In some aspects, the method further comprises providing a recovery period to the subject that begins upon completion of the administration in (a) and ends upon the start of the administration in (b). In some aspects, the recovery period is a duration sufficient for the subject to recover from an adverse event associated with the administration in (a). In some aspects, the adverse event is a serious adverse event. In some aspect, the recovery period is a duration sufficient for the subject to recover from toxicities associated with the CCRT other than fatigue, esophagitis, or alopecia. In some aspects, the PD-1 pathway inhibitor in (a) is administered prior to the CCRT. In some aspects, the recovery period begins upon completion of the CCRT. In some aspects, the chemotherapy of the CCRT is administered prior to the radiotherapy of the CCRT. In some aspects, the recovery period begins upon completion of the radiotherapy. In some aspects, the recovery period is from about 1 week to about 12 weeks, about 1 week to about 9 weeks, about 1 week to about 6 weeks, about 2 weeks to about 12 weeks, about 2 weeks to about 9 weeks, about 2 weeks to about 6 weeks, about 3 weeks to about 12 weeks, about 3 weeks to about 9 weeks, about 3 weeks to about 6 weeks, about 18 days from final administration of the PD-1 pathway inhibitor in (a) (e.g., from the last dose of the PD-1 pathway inhibitor when the administration in (a) comprises multiple doses of the PD-1 pathway inhibitor) to about 12 weeks, about 18 days from final administration of the PD-1 pathway inhibitor in (a) to about 9 weeks, or about 18 days from final administration of the PD-1 pathway inhibitor in (a) to about 6 weeks.

In some aspects, the method is a first line (1L) therapy.

In some aspects, the method is a second line (2L) therapy.

In some aspects, the method is a third line (3L) therapy.

In some aspects, the subject has progressed on a prior therapy (e.g., a standard of care therapy). Standard of care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard of care treatments for a wide variety of cancers. See NCCN GUIDELINES®, 2022-2023, https://www.nccn.org/guidelines/category_1, last accessed Dec. 20, 2022.

In some aspects, the lung cancer is recurrent following multi-modal therapy for locally advanced lung cancer.

In some aspects, the subject has not received a prior local or systemic anticancer therapy given as primary therapy for locally advanced disease.

In some aspects, the subject has not received a prior systemic therapy for cancer, the subject has not received a prior systemic therapy for lung cancer, or the subject has not received a prior systemic therapy for advanced or metastatic lung cancer.

In some aspects, the subject is naïve to prior immuno-oncology (I-O) therapy. In some aspects, the subject has never received I-O therapy, has received I-O therapy for a cancer other than lung cancer, or has received I-O therapy for a previous lung cancer but not a current lung cancer. In some aspects, the subject is naïve to prior I-O therapy, the subject is naïve to prior I-O therapy for lung cancer, or the lung cancer is naïve to prior I-O therapy. In some aspects, the prior I-O therapy is an antibody. In some aspects, the antibody binds to a checkpoint inhibitor. In some aspects, the prior I-O therapy is an anti-PD-1 antibody and/or the combination of an anti-PD-1 antibody and an anti-CTLA-4 antibody.

In some aspects, a method of the disclosure increases duration of progression-free survival (PFS), objective response rate (ORR), overall survival (OS), or any combination thereof as compared to a standard of care therapy (e.g., CCRT followed by maintenance therapy with durvalumab) and/or a prior therapy as disclosed herein.

In some aspects, a method of the disclosure reduces the size of a tumor, inhibits growth of a tumor, eliminates a tumor from the subject, prevents relapse of lung cancer, induces remission of lung cancer, provides a complete response or partial response, or any combination thereof.

In some aspects, the methods of the disclosure are administered to the subject based on the subject's performance status and/or cancer stage. Performance status and/or cancer stage can be indicated by any one or more systems in the art.

In some aspects, the lung cancer is unresectable, advanced, recurrent, and/or metastatic.

In some aspects, performance status is indicated by Eastern Cooperative Oncology Group performance status (ECOG PS), which utilizes standardized criteria for measuring how disease impacts a patient's daily living abilities. Example definitions for ECOG PS include: “0” for a patient who is fully active and able to carry on all pre-disease performance without restriction; “1” for a patient who is restricted in physically strenuous activity but ambulatory and able to carry out work of a light or sedentary nature; “2” for a patient who is ambulatory and capable of all self-care, up and about more than 50% of waking hours, but unable to carry out any work activities; “3” for a patient who is capable of only limited self-care and is confined to a bed or chair more than 50% of waking hours; and “4” for a patient who is completely disabled, cannot carry on any self-care, and is totally confined to bed or chair.

In some aspects the subject has an ECOG PS of 0, 1, 2, 3, or 4. In some aspects, the subject has an ECOG PS of ≤3. In some aspects, the subject has an ECOG PS of ≤2. In some aspects, the subject has an ECOG PS of ≤1.

In some aspects, lung cancer is staged based on a tumor (T)/node (N)/metastasis (M) staging system (T/N/M) such as the American Joint Committee on Cancer (AJCC) classification. See, e.g., https://www.cancer.org/cancer/lung-cancer/detection-diagnosis-staging/staging-nsclc.html, last accessed Dec. 20, 2022.

Stages for lung cancer (e.g., NSCLC) include: occult (hidden) stage, Stage 0 (carcinoma in situ), Stage I (e.g., Stage IA1, Stage IA2, Stage IA3, and Stage IB NSCLC), Stage II (e.g., Stage IIA and Stage IIB NSCLC), Stage III (e.g., Stage IIIA, Stage IIIB, and Stage IIIC NSCLC), and Stage IV (e.g., Stage IVA and Stage IVB NSCLC).

In some aspects the subject is afflicted with an occult (hidden) stage lung cancer (e.g., NSCLC). In the occult stage, the cancer cannot be seen by imaging or bronchoscopy (TX), has not spread to lymph nodes (NO), and has not metastasized (M0) (TX/N0/M0).

In some aspects the subject is afflicted with a Stage 0 lung cancer (e.g., NSCLC). In Stage 0 (Tis/N0/M0), cancer cells are found only in the lining of the airways and have not invaded deeper into other lung tissues (Tis). N0 and M0 are as described above.

In some aspects, the subject is afflicted with a Stage I lung cancer (e.g., NSCLC). Stage I lung cancer is divided, e.g., into Stage IA1, Stage IA2, Stage IA3, and Stage IB for NSCLC. N0 and M0 are as described above. In Stage IA1 (T1mi/N0/M0), the cancer is a minimally invasive adenocarcinoma, and the tumor is no larger than 3 centimeters (cm) across, with the part invading into deeper lung tissues not larger than 0.5 cm across (T1mi). In Stage IA1 (T1a/N0/M0), the tumor is not larger than 1 cm across, has not grown into the membranes surrounding the lungs, and does not affect the main branches of the bronchi (T1a). In Stage IA2 (T1b/N0/M0), the tumor is larger than 1 cm across but not larger than 2 cm across, has not grown into the membranes surrounding the lungs, and does not affect the main branches of the bronchi (T1b). In Stage IA3 (T1c/N0/M0), the tumor is larger than 2 cm across but not larger than 3 cm across, has not grown into the membranes surrounding the lungs, and does not affect the main branches of the bronchi (T1c). In Stage IB (T2a/N0/M0), one or more of the following is true: 1) the tumor is larger than 3 cm across but not larger than 4 cm across; 2) the cancer has spread to the main bronchus and is at least 2 cm below where the trachea joins the bronchus; 3) the cancer has spread to the innermost layer of the membrane that covers the lung and is not larger than 4 cm across; or 4) the tumor is not larger than 4 cm across but is partially clogging the airways (T2a).

In some aspects, the subject is afflicted with a Stage II lung cancer (e.g., NSCLC) Stage II is divided, e.g., into Stage IIA (T2b/N0/M0) and IIB (T1a/T1b/T1c/N1/M0 or T2a/T2b/N1/M0 or T3/N0/M0) for NSCLC. N0 and M0 are as described above. In Stage IIA, one or more of the following is true: 1) the tumor is larger than 4 cm across but not larger than 5 cm across; 2) the cancer has spread to the main bronchus, is at least 2 centimeters below where the trachea joins the bronchus, and the tumor is larger than 4 cm across but not larger than 5 cm across; 3) the cancer has spread to the innermost layer of the membrane that covers the lung, and the tumor is larger than 4 cm across but not larger than 5 cm across; or 4) the tumor is larger than 4 cm across but not larger than 5 cm across and is partially clogging the airways (T2b). In stage IIB, the cancer has either spread to the lymph nodes or not. If the cancer has spread to the lymph nodes, then the cancer can only have spread to the lymph nodes on the same side of the chest as the tumor, and the lymph nodes with cancer are within the lung or near the bronchus (N1). For T1a/T1b/T1c/N1/M0, the tumor is no larger than 3 cm across, has not grown into the membranes surrounding the lungs, and does not affect the main branches of the bronchi (T1a/T1b/T1c). For T2a/T2b/N1/M0, one or more of the following is true: 1) the tumor is larger than 3 cm across but not larger than 5 cm across; 2) the cancer has spread to the main bronchus, is at least 2 centimeters below where the trachea joins the bronchus, and the tumor is not larger than 5 cm across; 3) the cancer has spread to the innermost layer of the membrane that covers the lung and is not larger than 5 cm across; or 4) the tumor is not larger than 5 cm across and is partially clogging the airways (T2a/T2b). In T3/N0/M0, the cancer has not spread to the lymph nodes or metastasized but one or more of the following is true: 1) the cancer is larger than 5 cm across but not larger than 7 cm across; 2) the cancer has grown into the chest wall, the parietal pleura, the phrenic nerve, or the parietal pericardium; or 3) the same lobe of a lung has 2 or more separate tumor nodules (T3).

In some aspects, the subject is afflicted with a Stage III lung cancer (e.g., NSCLC). Stage III is divided, e.g., into Stage IIIA (T1a/T1b/T1c/N2/M0 or T2a/T2b/N2/M0 or T3/N1/M0 or T4/N0 or N1/M0), IIIB (T1a/T1b/T1c/N3/M0 or T2a/T2b/N3/M0 or T3/N2/M0 or T4/N2/M0), and IIIC (T3/N3/M0 or T4/N3/M0) for NSCLC. T1a/T1b/T1c, T2a/T2b, T3, N0, N1, and M0 are as described above. For N2, the cancer has spread to lymph nodes around the carina or in the mediastinum on the same side as the tumor. For N3, the cancer has spread to lymph nodes on either side of the body near the collarbone, and/or has spread on the other side of the body from the main tumor to hilar or mediastinal lymph nodes. For T4, one or more of the following is true: 1) the tumor is larger than 7 cm across; 2) the tumor has grown into the mediastinum, the heart, the large blood vessels near the heart (e.g., the aorta), the trachea, the esophagus, the diaphragm, the backbone, or the carina; or 3) 2 or more tumor nodules are present in different lobes of the same lung.

In some aspects, the subject is afflicted with a Stage IV lung cancer (e.g., NSCLC). Stage IV is divided, e.g., into Stage IVA (Any T/Any N/M1a or Any T/Any N/M1b) and IVB (Any T/Any N/M1c) for NSCLC. For Any T, the tumor can be of any size and can have grown into nearby structures or not have grown into them. For Any N, the cancer can have reached nearby lymph nodes or not have reached them. For M1a, one or more of the following is true: 1) the cancer has spread to both lungs; 2) cancer cells are found in the fluid around the lung; 3) cancer cells are found in the fluid around the heart. For M1b, the cancer has spread as a single tumor to a distant lymph node or another organ (e.g., the liver, bones, or brain). For M1c, the cancer has spread as two or more tumors to distant lymph nodes and/or another organ (e.g., the liver, bones, or brain).

In some aspects, the lung cancer is small cell lung cancer (SCLC). In some aspects, staging of the SCLC is by T/N/M staging. In some aspects, rather than T/N/M staging, the SCLC is staged as either limited stage or extensive stage. Limited stage SCLC is confined to one lung and/or the local lymph nodes. Extensive stage SCLC is found in both lungs and/or distant sites in the body.

In some aspects, the lung cancer is non-small cell lung cancer (NSCLC). NSCLC includes NSCLC with a histology that is “not otherwise specified” (NOS), NSCLC with a squamous histology (SQ), and NSCLC with a non-squamous histology (NSQ, including adenocarcinoma, large cell, and undifferentiated carcinoma). In some aspects, the NSCLC has a squamous histology. In some aspects, the NSCLC has a non-squamous histology. In some aspects, staging of the NSCLC is by T/N/M staging. In some aspects, the NSCLC comprises locally advanced Stage IIIA, IIIB, or IIIC NSCLC. In some aspects, the subject is free of progressive lung cancer during the CCRT or the recovery period of a method as disclosed herein.

Surgery (i.e., surgical resection), radiotherapy (RT, also interchangeably referred to herein as radiation therapy), and chemotherapy are three modalities commonly used to treat NSCLC patients. As a class, NSCLCs are relatively insensitive to chemotherapy and RT, compared to small cell carcinoma. In general, for patients with Stage I or II disease, surgical resection has provided the best chance for cure, with chemotherapy often used both pre-operatively and post-operatively. RT can also be used as adjuvant therapy for patients with respectable NSCLC, the primary local treatment, or as palliative therapy for patients with incurable NSCLC. Patients with advanced or metastatic disease (e.g., Stage IV NSCLC) who have a good performance status (PS) can benefit from chemotherapy.

Specific targeted therapies have also been developed for the treatment of advanced or metastatic NSCLC in subjects with sensitizing mutations in genes for epidermal growth factor receptor (EGFR), anaplastic lymphoma kinase (ALK), ROS-1, neurotrophin receptor tyrosine kinase (NTRK), and B-rapidly accelerated fibrosarcoma proto-oncogene (BRAF, e.g., the BRAF V600E mutation).

In some aspects, the subject has an EGFR, ALK, NTRK, ROS-1, or BRAF mutation sensitive to targeted inhibitor therapy.

In some aspects, the subject has no EGFR, ALK, NTRK, ROS-1, or BRAF mutation sensitive to targeted inhibitor therapy.

### II.A PD-1 Pathway Inhibitors

In some aspects, the PD-1 pathway inhibitor administered with the CCRT in the disclosed methods is the same as the PD-1 pathway inhibitor administered with the LAG-3 antagonist in the disclosed methods.

In some aspects, the PD-1 pathway inhibitor administered with CCRT in the disclosed methods is different from the PD-1 pathway inhibitor administered with the LAG-3 antagonist in the disclosed methods.

In some aspects, the PD-1 pathway inhibitor administered with the CCRT and/or the LAG-3 antagonist comprises a PD-1 inhibitor and/or a PD-L1 inhibitor. In some aspects, the PD-1 inhibitor and/or PD-L1 inhibitor comprises a small molecule. In some aspects, the PD-1 inhibitor and/or PD-L1 inhibitor comprises a millamolecule. In some aspects, the PD-1 inhibitor and/or PD-L1 inhibitor comprises a macrocyclic peptide. In certain aspects, the PD-1 inhibitor and/or PD-L1 inhibitor comprises BMS-986189. In some aspects, the PD-1 inhibitor comprises an inhibitor disclosed in International Publication No. WO2014/151634, which is incorporated by reference herein in its entirety. In some aspects, the PD-1 inhibitor is INCMGA00012 (Insight Pharmaceuticals). In some aspects, the PD-1 inhibitor comprises a combination of an anti-PD-1 antibody disclosed herein and a PD-1 small molecule inhibitor. In some aspects, the PD-L1 inhibitor comprises a millamolecule having a formula set forth in formula (I):

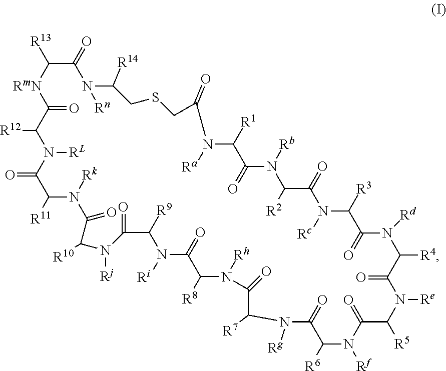

wherein R¹-R¹³ are amino acid side chains, R^{a}-Rⁿ are hydrogen, methyl, or form a ring with a vicinal R group, and R¹⁴ is —C(O)NHR¹⁵, wherein R¹⁵ is hydrogen, or a glycine residue optionally substituted with additional glycine residues and/or tails which can improve pharmacokinetic properties. In some aspects, the PD-L1 inhibitor comprises a compound disclosed in International Publication No. WO2014/151634, which is incorporated by reference herein in its entirety. In some aspects, the PD-L1 inhibitor comprises a compound disclosed in International Publication No. WO2016/039749, WO2016/149351, WO2016/077518, WO2016/100285, WO2016/100608, WO2016/126646, WO2016/057624, WO2017/151830, WO2017/176608, WO2018/085750, WO2018/237153, or WO2019/070643, each of which is incorporated by reference herein in its entirety. In some aspects, the PD-L1 inhibitor comprises a small molecule PD-L1 inhibitor disclosed in International Publication No. WO2015/034820, WO2015/160641, WO2018/044963, WO2017/066227, WO2018/009505, WO2018/183171, WO2018/118848, WO2019/147662, or WO2019/169123, each of which is incorporated by reference herein in its entirety.

In some aspects, the PD-1 pathway inhibitor administered with the CCRT and/or the LAG-3 antagonist comprises a soluble PD-L2 polypeptide. In some aspects, the soluble PD-L2 polypeptide comprises a fusion polypeptide. In some aspects, the soluble PD-L2 polypeptide comprises a ligand binding fragment of the PD-L2 extracellular domain. In some aspects, the soluble PD-L2 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble PD-L2 polypeptide is AMP-224 (see, e.g., US 2013/0017199).

In some aspects, the PD-1 pathway inhibitor administered with the CCRT and/or the LAG-3 antagonist comprises an anti-PD-1 antibody and/or an anti-PD-L1 antibody.

In some aspects, the PD-1 pathway inhibitor administered with the CCRT and/or the LAG-3 antagonist is administered at a flat dose.

In some aspects, the PD-1 pathway inhibitor administered with the CCRT and/or the LAG-3 antagonist is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the PD-1 pathway inhibitor administered with the CCRT and/or the LAG-3 antagonist is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the PD-1 pathway inhibitor administered with the CCRT and/or the LAG-3 antagonist is administered as a weight-based dose.

In some aspects, the PD-1 pathway inhibitor administered with the CCRT and/or the LAG-3 antagonist is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the PD-1 pathway inhibitor administered with the CCRT and/or the LAG-3 antagonist is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose of the PD-1 pathway inhibitor administered with the CCRT is different from the dose of the PD-1 pathway inhibitor administered with the LAG-3 antagonist.

In some aspects, the dose of the PD-1 pathway inhibitor administered with the CCRT and/or the LAG-3 antagonist is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the PD-1 pathway inhibitor is administered with a CCRT as disclosed herein for three 21-day cycles.

In some aspects, the PD-1 pathway inhibitor is administered with a LAG-3 antagonist as disclosed herein once about every four weeks as a maintenance therapy.

### II.A.1 Anti-PD-1 Antibodies

Anti-PD-1 antibodies that are known in the art can be used in the methods of the disclosure. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies that can be used in the methods of the disclosure have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

Anti-PD-1 antibodies that can be used in the methods of the disclosure include nivolumab (also known as OPDIVO®, 5C4, BMS-936558, MDX-1106, and ONO-4538), pembrolizumab (Merck; also known as KEYTRUDA®, lambrolizumab, and MK3475; see WO 2008/156712), PDR001 (Novartis; also known as spartalizumab; see WO 2015/112900 and U.S. Pat. No. 9,683,048), MEDI-0680 (AstraZeneca; also known as AMP-514; see WO 2012/145493), TSR-042 (Tesaro Biopharmaceutical; also known as ANB011 or dostarlimab; see WO 2014/179664), cemiplimab (Regeneron; also known as LIBTAYO® or REGN2810; see WO 2015/112800 and U.S. Pat. No. 9,987,500), JS001 (TAIZHOU JUNSHI PHARMA; also known as toripalimab; see Si-Yang Liu et al., J. Hematol. Oncol. 10:136 (2017)), PF-06801591 (Pfizer; also known as sasanlimab; US 2016/0159905), BGB-A317 (Beigene; also known as tislelizumab; see WO 2015/35606 and US 2015/0079109), BI 754091 (Boehringer Ingelheim; see Zettl M et al., Cancer. Res. (2018); 78 (13 Suppl): Abstract 4558), INCSHR1210 (Jiangsu Hengrui Medicine; also known as SHR-1210 or camrelizumab; see WO 2015/085847; Si-Yang Liu et al., J. Hematol. Oncol. 10:136 (2017)), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10 (2): 183-203 (2018), IBI308 (Innovent; also known as sintilimab; see WO 2017/024465, WO 2017/025016, WO 2017/132825, and WO 2017/133540), and SSI-361 (Lyvgen Biopharma Holdings Limited, US 2018/0346569).

Anti-PD-1 antibodies that can be used in the methods of the disclosure also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab.

In some aspects, the antibodies that cross-compete for binding to human PD-1 with, or bind to the same epitope region as, any anti-PD-1 antibody disclosed herein, e.g., nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

The ability of antibodies to cross-compete for binding to an antigen indicates that the antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region. Cross-competing antibodies can be readily identified based on their ability to cross-compete in standard binding assays such as Biacore analysis, ELISA assays or flow cytometry (see, e.g., WO 2013/173223).

Anti-PD-1 antibodies that can be used in the methods of the disclosure also include antigen-binding portions of any of the above full-length antibodies. It has been amply demonstrated that the antigen-binding function of an antibody can be performed by fragments of a full-length antibody.

Anti-PD-1 antibodies that can be used in the methods of the disclosure are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

In some aspects, the anti-PD-1 antibody comprises a full-length antibody.

In some aspects, the anti-PD-1 antibody comprises a monoclonal, human, humanized, chimeric, or multispecific antibody. In some aspects, the multispecific antibody comprises a dual-affinity re-targeting antibody (DART), a DVD-Ig, or bispecific antibody.

In some aspects, the anti-PD-1 antibody comprises a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-1 antibody comprises nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or an antigen binding portion thereof.

In some aspects, the anti-PD-1 antibody is formulated for intravenous administration.

In some aspects, the anti-PD-1 antibody is administered intravenously for about 30 minutes.

In some aspects, the anti-PD-1 antibody comprises nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol. Res. 2 (9): 846-56).

In some aspects, nivolumab is administered at a flat dose of about 240 mg once about every 2 weeks. In some aspects, nivolumab is administered at a flat dose of about 240 mg once about every 3 weeks. In some aspects, nivolumab is administered at a flat dose of about 360 mg once about every 3 weeks. In some aspects, nivolumab is administered at a flat dose of about 480 mg once about every 4 weeks.

In some aspects, nivolumab is administered intravenously at a dose of about 240 mg for about 30 minutes on Day 1 of a two-week cycle.

In some aspects, nivolumab is administered intravenously at a dose of about 360 mg for about 30 minutes on Day 1 of a three-week cycle.

In some aspects, nivolumab is administered intravenously at a dose of about 480 mg for about 30 minutes on Day 1 of a four-week cycle.

In some aspects, the methods of the disclosure comprise an anti-PD-1 antibody comprising CDR1, CDR2 and CDR3 domains of the heavy chain variable region having the sequence set forth in SEQ ID NO: 13, and CDR1, CDR2 and CDR3 domains of the light chain variable region having the sequence set forth in SEQ ID NO:14.

In some aspects, the methods of the disclosure comprise an anti-PD-1 antibody comprising: (a) a heavy chain variable region CDR1 comprising the sequence set forth in SEQ ID NO:15; (b) a heavy chain variable region CDR2 comprising the sequence set forth in SEQ ID NO:16; (c) a heavy chain variable region CDR3 comprising the sequence set forth in SEQ ID NO:17; (d) a light chain variable region CDR1 comprising the sequence set forth in SEQ ID NO:18; (e) a light chain variable region CDR2 comprising the sequence set forth in SEQ ID NO:19; and (f) a light chain variable region CDR3 comprising the sequence set forth in SEQ ID NO:20.

In some aspects, the methods of the disclosure comprise an anti-PD-1 antibody comprising heavy and light chain variable regions comprising the sequences set forth in SEQ ID NOs: 13 and 14, respectively.

In some aspects, the methods of the disclosure comprise an anti-PD-1 antibody comprising heavy and light chains comprising the sequences as set forth in SEQ ID NOs: 11 and 12, respectively.

In some aspects, the anti-PD-1 antibody comprises pembrolizumab. Pembrolizumab is a humanized monoclonal IgG4 (S228P) antibody directed against human cell surface receptor PD-1. Pembrolizumab is described, for example, in U.S. Pat. Nos. 8,354,509 and 8,900,587.

In some aspects, pembrolizumab is administered at a flat dose of about 200 mg once about every 2 weeks. In some aspects, pembrolizumab is administered at a flat dose of about 200 mg once about every 3 weeks. In some aspects, pembrolizumab is administered at a flat dose of about 400 mg once about every 6 weeks. In some aspects, pembrolizumab is administered at a flat dose of about 300 mg once about every 4-5 weeks.

In some aspects, pembrolizumab is administered intravenously at a dose of about 200 mg on Day 1, then once about every 3 weeks.

In some aspects, the methods of the disclosure comprise an anti-PD-1 antibody comprising CDR1, CDR2 and CDR3 domains of the heavy chain variable region having the sequence set forth in SEQ ID NO: 79, and CDR1, CDR2 and CDR3 domains of the light chain variable region having the sequence set forth in SEQ ID NO:80.

In some aspects, the methods of the disclosure comprise an anti-PD-1 antibody comprising: (a) a heavy chain variable region CDR1 comprising the sequence set forth in SEQ ID NO:81; (b) a heavy chain variable region CDR2 comprising the sequence set forth in SEQ ID NO:82; (c) a heavy chain variable region CDR3 comprising the sequence set forth in SEQ ID NO:83; (d) a light chain variable region CDR1 comprising the sequence set forth in SEQ ID NO:84; (e) a light chain variable region CDR2 comprising the sequence set forth in SEQ ID NO:85; and (f) a light chain variable region CDR3 comprising the sequence set forth in SEQ ID NO:86.

In some aspects, the methods of the disclosure comprise an anti-PD-1 antibody comprising heavy and light chain variable regions comprising the sequences set forth in SEQ ID NOs: 79 and 80, respectively.

In some aspects, the methods of the disclosure comprise an anti-PD-1 antibody comprising heavy and light chains comprising the sequences as set forth in SEQ ID NOs: 77 and 78, respectively.

In some aspects, the anti-PD-1 antibody comprises cemiplimab (REGN2810). Cemiplimab is described, for example, in WO 2015/112800 and U.S. Pat. No. 9,987,500.

In some aspects, cemiplimab is administered intravenously at a dose of about 3 mg/kg or about 350 mg once about every 3 weeks.

In some aspects, the methods of the disclosure comprise an anti-PD-1 antibody comprising CDR1, CDR2 and CDR3 domains of the heavy chain variable region having the sequence set forth in SEQ ID NO:35, and CDR1, CDR2 and CDR3 domains of the light chain variable region having the sequence set forth in SEQ ID NO:36.

In some aspects, the methods of the disclosure comprise an anti-PD-1 antibody comprising: (a) a heavy chain variable region CDR1 comprising the sequence set forth in SEQ ID NO:37; (b) a heavy chain variable region CDR2 comprising the sequence set forth in SEQ ID NO:38; (c) a heavy chain variable region CDR3 comprising the sequence set forth in SEQ ID NO:39; (d) a light chain variable region CDR1 comprising the sequence set forth in SEQ ID NO:40; (e) a light chain variable region CDR2 comprising the sequence set forth in SEQ ID NO:41; and (f) a light chain variable region CDR3 comprising the sequence set forth in SEQ ID NO:42.

In some aspects, the methods of the disclosure comprise an anti-PD-1 antibody comprising heavy and light chain variable regions comprising the sequences set forth in SEQ ID NOs: 35 and 36, respectively.

In some aspects, the methods of the disclosure comprise an anti-PD-1 antibody comprising heavy and light chains comprising the sequences as set forth in SEQ ID NOs: 33 and 34, respectively.

In some aspects, the anti-PD-1 antibody comprises spartalizumab (PDR001). Spartalizumab is described, for example, in WO 2015/112900 and U.S. Pat. No. 9,683,048.

In some aspects, spartalizumab is administered intravenously at a dose of about 300 mg once about every 3 weeks or 400 mg once about every 4 weeks.

In some aspects, the methods of the disclosure comprise an anti-PD-1 antibody comprising CDR1, CDR2 and CDR3 domains of the heavy chain variable region having the sequence set forth in SEQ ID N0.59, and CDR1, CDR2 and CDR3 domains of the light chain variable region having the sequence set forth in SEQ ID NO:60.

In some aspects, the methods of the disclosure comprise an anti-PD-1 antibody comprising: (a) a heavy chain variable region CDR1 comprising the sequence set forth in SEQ ID NO:61; (b) a heavy chain variable region CDR2 comprising the sequence set forth in SEQ ID NO:62; (c) a heavy chain variable region CDR3 comprising the sequence set forth in SEQ ID NO:63; (d) a light chain variable region CDR1 comprising the sequence set forth in SEQ ID NO:64; (e) a light chain variable region CDR2 comprising the sequence set forth in SEQ ID NO:65; and (f) a light chain variable region CDR3 comprising the sequence set forth in SEQ ID NO:66.

In some aspects, the methods of the disclosure comprise an anti-PD-1 antibody comprising heavy and light chain variable regions comprising the sequences set forth in SEQ ID NOs: 59 and 60, respectively.

In some aspects, the methods of the disclosure comprise an anti-PD-1 antibody comprising heavy and light chains comprising the sequences as set forth in SEQ ID NOs: 57 and 58, respectively.

### II.A.2 Anti-PD-L1 Antibodies

Anti-PD-L1 antibodies that are known in the art can be used in the methods of the disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

Anti-PD-L1 antibodies that can be used in the methods of the disclosure include BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31 (suppl): 3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl): TPS3113 (2018)), ICO 36, FAZ053 (Novartis), and CK-301 (Checkpoint Therapeutics; see Gorelik et al., AACR: Abstract 4606 (April 2016)).

Anti-PD-L1 antibodies that can be used in the methods of the disclosure also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. In certain aspects, the antibodies that cross-compete for binding to human PD-L1 with, or bind to the same epitope region as, any anti-PD-L1 antibody disclosed herein, e.g., atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies that can be used in the methods of the disclosure also include antigen-binding portions of any of the above full-length antibodies.

Anti-PD-L1 antibodies that can be used in the methods of the disclosure are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

In some aspects, an anti-PD-L1 antibody is substituted for the anti-PD-1 antibody in any of the methods disclosed herein.

In some aspects, the anti-PD-L1 antibody comprises a full-length antibody.

In some aspects, the anti-PD-L1 antibody comprises a monoclonal, human, humanized, chimeric, or multispecific antibody. In some aspects, the multispecific antibody comprises a DART, a DVD-Ig, or bispecific antibody.

In some aspects, the anti-PD-L1 antibody comprises a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-L1 antibody comprises BMS-936559, atezolizumab, durvalumab, avelumab, STI-1014, CX-072, KN035, LY3300054, BGB-A333, ICO 36, FAZ053, CK-301, or an antigen binding portion thereof.

In some aspects, the PD-L1 antibody comprises atezolizumab. Atezolizumab is a fully humanized IgG1 monoclonal anti-PD-L1 antibody. In some aspects, atezolizumab is administered as a flat dose of about 800 mg once about every 2 weeks. In some aspects, atezolizumab is administered as a flat dose of about 840 mg once about every 2 weeks.

In some aspects, atezolizumab is administered intravenously at a dose of about 1,200 mg on Day 1 of a three-week cycle.

In some aspects, the PD-L1 antibody comprises durvalumab. Durvalumab is a human IgG1 kappa monoclonal anti-PD-L1 antibody. In some aspects, durvalumab is administered at a dose of about 10 mg/kg once about every 2 weeks. In some aspects, durvalumab is administered at a dose of about 10 mg/kg once about every 2 weeks for up to 12 months. In some aspects, durvalumab is administered as a flat dose of about 800 mg/kg once about every 2 weeks. In some aspects, durvalumab is administered as a flat dose of about 1200 mg/kg once about every 3 weeks.

In some aspects, the PD-L1 antibody comprises avelumab. Avelumab is a human IgG1 lambda monoclonal anti-PD-L1 antibody. In some aspects, avelumab is administered as a flat dose of about 800 mg once about every 2 weeks.

### II.B CCRT

Concurrent chemoradiotherapies (CCRT) that are known in the art as well as CCRTs disclosed herein can be used in the disclosed methods.

In some aspects, the CCRT comprises a platinum doublet chemotherapy (PDCT).

In some aspects, the PDCT comprises a platinum agent in combination with a nucleoside analog, an antimetabolite, a taxane, a vinca alkaloid, or a topisomerase inhibitor.

In some aspects, the platinum agent comprises cisplatin, carboplatin, oxaliplatin, satraplatin, picoplatin, nedaplatin, triplatin, lipoplatin, or phenanthriplatin. In some aspects, the platinum agent comprises cisplatin. In some aspects, the platinum agent comprises carboplatin.

In some aspects, the nucleoside analog comprises cytarabine, gemcitabine, lamivudine, entecavir, or telbivudine. In some aspects, the nucleoside analog comprises gemcitabine.

In some aspects, the antimetabolite comprises capecitabine, cladribine, clofarabine, cytarabine, floxuridine, fludarabine, fluorouracil, mercaptopurine, methotrexate, pemetrexed, pentostatin, pralatrexate, or thioguanine. In some aspects, the antimetabolite comprises pemetrexed.

In some aspects, the taxane comprises paclitaxel, albumin-bound paclitaxel (also called nab-paclitaxel), docetaxel, or cabazitaxel. In some aspects, the taxane comprises paclitaxel.

In some aspects, the vinca alkaloid comprises vinblastine, vincristine, vinorelbine, vindesine, vincaminol, vineridine, or vinburnine. In some aspects, the vinca alkaloid comprises vinorelbine or vinblastine.

In some aspects, the topoisomerase inhibitor comprises etoposide, mitoxantrone, doxorubicin, irinotecan, topotecan, or camptothecin. In some aspects, the topoisomerase inhibitor comprises etoposide. In some aspects, the topoisomerase inhibitor comprises irinotecan.

In some aspects, the PDCT is administered for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 weeks.

In some aspects, the PDCT is administered about every three weeks for about 1, about 2, about 3, about 4, about 5, about 6, about 7, or about 8 cycles. In some aspects, the PDCT is administered about every three weeks for about 1, about 2, about 3, about 4, about 5, or about 6 cycles. In some aspect, the PDCT is administered about every three weeks for about 1, about 2, about 3, or about 4 cycles. In some aspect, the PDCT is administered for three 21-day cycles.

In some aspects, the platinum agent comprises cisplatin. In some aspects, cisplatin is administered at a dose of about 25 mg/m² to about 150 mg/m², about 50 mg/m² to about 100 mg/m², about 75 mg/m² to about 100 mg/m², or about 75 mg/m² to about 80 mg/m². In some aspects, cisplatin is administered at a dose of about 50 mg/m², about 55 mg/m², about 60 mg/m², about 65 mg/m², about 70 mg/m², about 75 mg/m², about 76 mg/m², about 77 mg/m², about 78 mg/m², about 79 mg/m², about 80 mg/m², about 85 mg/m², about 90 mg/m², about 95 mg/m², or about 100 mg/m². In some aspects, cisplatin is administered intravenously for about 60 minutes. In some aspects, cisplatin is administered on Day 1 of each of three 21-day cycles.

In some aspects, the platinum agent comprises carboplatin. In some aspects, carboplatin is administered at a dose for a target area under the concentration-time curve (AUC) of about 1 mg/mL·min to about 10 mg/mL·min. In some aspects, carboplatin is administered in a dose for a target AUC of about 1 mg/mL·min, about 2 mg/mL·min, about 3 mg/mL·min, about 4 mg/mL·min, about 5 mg/mL·min, about 6 mg/mL·min, about 7 mg/mL·min, about 8 mg/mL·min, about 9 mg/mL·min, or about 10 mg/mL·min. In some aspects, carboplatin is administered in a dose for a target AUC of about 2 mg/mL·min. In some aspects, carboplatin is administered in a dose for a target AUC of about 5 mg/mL·min or about 6 mg/mL·min. In some aspects, carboplatin is administered in a dose for a target AUC of about 5 mg/mL·min. In some aspects, carboplatin is administered in a dose for a target AUC of about 6 mg/mL·min. In some aspects, carboplatin is administered intravenously for about 30 minutes. In some aspects, carboplatin is administered on Day 1 of each of three 21-day cycles. In some aspects, carboplatin is administered on Day 1 of a first 21-day cycle and on Days 1, 8, and 15 of each of a second and third 21-day cycle.

Carboplatin dose can be calculated according to methods known in the art. In some aspects, carboplatin dose is calculated using the Calvert formula as follows:

Carboplatin dose (mg)=target AUC×(CrCl [mL/min]+25).
The Creatine Clearance (CrCl) calculation in the Calvert formula can be determined using the Cockcroft-Gault formula:

Cockcroft-Gault CrCl=[(140−age)×(Weight in kg)×(0.85 if female)]/(72×Cr).
The Cockcroft-Gault formula includes a subject's most recent weight (kg) and most recent serum creatinine (Cr) concentration (mg/dL). In some aspects, if calculation of CrCl by the Cockcroft-Gault formula yields a result of >125 mL/min, then a CrCl is calculated by an alternative formula per institutional standards or capped at 125 mL/min.

In some aspects, the PDCT comprises cisplatin or carboplatin in combination with gemcitabine, paclitaxel, albumin-bound paclitaxel, docetaxel, pemetrexed, vinorelbine, vinblastine, etoposide, or irinotecan.

In some aspects, the PDCT comprises cisplatin or carboplatin in combination with gemcitabine. In some aspects, gemcitabine is administered at a dose of about 1,000 mg/m² to about 1,250 mg/m². In some aspects, gemcitabine is administered at a dose of about 1,000 mg/m², about 1,050 mg/m², about 1,100 mg/m², about 1,150 mg/m², about 1,200 mg/m², or about 1,250 mg/m². In some aspects, gemcitabine is administered intravenously over about 30 minutes. In some aspects, gemcitabine is administered on Days 1, 8, and 15 of a three-week cycle for up to about 3, about 4, about 5, or about 6 cycles. In some aspects, gemcitabine is administered on Days 1 and 8 of a three-week cycle for up to about 3, about 4, about 5, or about 6 cycles. In some aspects, the PDCT comprises a dose of about 1,000 mg/m² to about 1,250 mg/m² gemcitabine administered on Days 1 and 8 of a three-week cycle for about 3 cycles and a dose of about 75 mg/m² to about 80 mg/m² cisplatin administered on Day 1 of each cycle. In some aspects, gemcitabine is administered intravenously over about 30 minutes and cisplatin is administered intravenously over about 60 minutes. In some aspects, the PDCT comprises a dose of about 1,000 mg/m² gemcitabine administered on Days 1, 8, and 15 of a three-week cycle for about 3 cycles and carboplatin administered in a dose for a target AUC of about 5 mg/mL·min on Day 1 of each cycle. In some aspects, gemcitabine and carboplatin are each administered intravenously over about 30 minutes.

In some aspects, the PDCT comprises cisplatin or carboplatin in combination with paclitaxel or albumin-bound paclitaxel.

In some aspects, the PDCT comprises cisplatin or carboplatin in combination with paclitaxel. In some aspects, paclitaxel is administered at a dose of about 45 mg/m² to about 200 mg/m². In some aspects, paclitaxel is administered at a dose of about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², or about 200 mg/m². In some aspects, paclitaxel is administered intravenously over about 60 minutes to about 180 minutes. In some aspects, the PDCT comprises a dose of about 175 mg/m² or about 200 mg/m² paclitaxel administered on Day 1 of a three-week cycle for about 3 cycles and a dose of about 75 mg/m² to about 80 mg/m² cisplatin administered on Day 1 of each cycle. In some aspects, the PDCT comprises a dose of about 135 mg/m² paclitaxel administered on Day 1 of a three-week cycle for about 3 cycles and a dose of about 75 mg/m² cisplatin administered on Day 1 of each cycle. In some aspects, paclitaxel is administered intravenously over about 180 minutes and cisplatin is administered intravenously over about 60 minutes. In some aspects, the PDCT comprises a dose of about 175 mg/m² or about 200 mg/m² paclitaxel administered on Day 1 of a first three-week cycle and carboplatin administered in a dose for a target AUC of about 5 mg/mL·min or about 6 mg/mL·min on Day 1 of the cycle. In some aspects, paclitaxel is administered intravenously over about 180 minutes and carboplatin is administered intravenously over about 30 minutes in the first cycle. In some aspects, the PDCT comprises a dose of about 45 mg/m² or about 50 mg/m² paclitaxel administered on Days 1, 8, and 15 of a second and third three-week cycle and carboplatin administered in a dose for a target AUC of about 2 mg/mL·min on Days 1, 8, and 15 of each of the second and third cycles. In some aspects, paclitaxel is administered intravenously over about 60 minutes and carboplatin is administered intravenously over about 30 minutes in the second and third cycles. In some aspects, the carboplatin is administered after the paclitaxel in each cycle.

In some aspects, the PDCT comprises cisplatin or carboplatin in combination with albumin-bound paclitaxel. In some aspects, albumin-bound paclitaxel is administered at a dose of about 100 mg/m². In some aspects, albumin-bound paclitaxel is administered intravenously over about 30 minutes. In some aspects, the PDCT comprises a dose of about 100 mg/m² albumin-bound paclitaxel administered on Days 1, 8, and 15 of a three-week cycle for about 3 cycles and a dose of about 75 mg/m² to about 80 mg/m² cisplatin administered on Day 1 of each cycle. In some aspects, albumin-bound paclitaxel is administered intravenously over about 30 minutes and cisplatin is administered intravenously over about 60 minutes. In some aspects, the PDCT comprises a dose of about 100 mg/m² albumin-bound paclitaxel administered on Days 1, 8, and 15 of a three-week cycle for about 3 cycles and carboplatin administered in a dose for a target AUC of about 6 mg/mL·min on Day 1 of each cycle. In some aspects, albumin-bound paclitaxel and carboplatin are each administered intravenously over about 30 minutes.

In some aspects, the PDCT comprises cisplatin or carboplatin in combination with docetaxel. In some aspects, docetaxel is administered at a dose of about 75 mg/m². In some aspects, docetaxel is administered intravenously over about 60 minutes. In some aspects, the PDCT comprises a dose of about 75 mg/m² docetaxel administered on Day 1 of a three-week cycle for about 3 cycles and a dose of about 75 mg/m² cisplatin administered on Day 1 of each cycle. In some aspects, docetaxel and cisplatin are each administered intravenously over about 60 minutes. In some aspects, the PDCT comprises a dose of about 75 mg/m² docetaxel administered on Day 1 of a three-week cycle for about 3 cycles and carboplatin administered in a dose for a target AUC of about 6 mg/mL·min on Day 1 of each cycle. In some aspects, docetaxel is administered intravenously over about 60 minutes and carboplatin is administered intravenously over about 30 minutes.

In some aspects, the PDCT comprises cisplatin or carboplatin in combination with pemetrexed. In some aspects, pemetrexed is administered at a dose of about 500 mg/m². In some aspects, pemetrexed is administered intravenously over about 10 minutes. In some aspects, the PDCT comprises a dose of about 500 mg/m² pemetrexed administered on Day 1 of a three-week cycle for about 3 cycles and a dose of about 75 mg/m² cisplatin administered on Day 1 of each cycle. In some aspects, pemetrexed is administered intravenously over about 10 minutes and cisplatin is administered intravenously over about 60 minutes. In some aspects, cisplatin is administered after pemetrexed. In some aspects, the cisplatin is replaced with carboplatin if the cisplatin is not tolerated by the subject. In some aspects, the PDCT comprises a dose of about 500 mg/m² pemetrexed administered on Day 1 of a three-week cycle for about 3 cycles and carboplatin administered in a dose for a target AUC of about 5 mg/mL·min on Day 1 of each cycle. In some aspects, pemetrexed is administered intravenously over about 10 minutes and carboplatin is administered intravenously over about 30 minutes. In some aspects, carboplatin is administered after pemetrexed. In some aspects, the pemetrexed is replaced with etoposide if the pemetrexed is not tolerated by the subject.

In some aspects, the PDCT comprises cisplatin or carboplatin in combination with etoposide. In some aspects, etoposide is administered at a dose of about 50 mg/m² to about 100 mg/m². In some aspects, etoposide is administered intravenously over about 30 minutes to about 60 minutes. In some aspects, the PDCT comprises a dose of about 100 mg/m² etoposide administered on Days 1, 2, and 3 of a three-week cycle for about 3 cycles and a dose of about 80 mg/m² cisplatin administered on Day 1 of each cycle. In some aspects, etoposide and cisplatin are each administered intravenously over about 60 minutes. In some aspects, cisplatin is administered after etoposide. In some aspects, the PDCT comprises a dose of about 100 mg/m² etoposide administered on Days 1, 2, and 3 of a three-week cycle for about 3 cycles and carboplatin administered in a dose for a target AUC of about 5 mg/mL·min on Day 1 of each cycle. In some aspects, etoposide and carboplatin are each administered intravenously over about 30 minutes. In some aspects, carboplatin is administered after etoposide.

In some aspects, the PDCT comprises cisplatin and vinorelbine. In some aspects, vinorelbine is administered at a dose of about 25 mg/m² to about 30 mg/m². In some aspects, vinorelbine is administered intravenously over about 5 minutes to about 10 minutes. In some aspects, the PDCT comprises a dose of about 25 mg/m² to about 30 mg/m² vinorelbine administered on Days 1 and 8 of a three-week cycle for about 3 cycles and a dose of about 75 mg/m² to about 80 mg/m² cisplatin administered on Day 1 of each cycle. In some aspects, vinorelbine is administered intravenously over about 5 minutes to about 10 minutes and cisplatin is administered intravenously over about 60 minutes. In some aspects, the PDCT comprises a dose of about 25 mg/m² vinorelbine administered on Days 1, 8, 15, and 22 of a three-week cycle for about 3 cycles and a dose of about 50 mg/m² cisplatin administered on Days 1 and 8 of each cycle. In some aspects, vinorelbine is administered intravenously over about 5 minutes to about 10 minutes and cisplatin is administered intravenously over about 60 minutes. In some aspects, the PDCT comprises a dose of about 30 mg/m² vinorelbine administered on Days 1, 8, 15, and 22 of a three-week cycle for about 3 cycles and a dose of about 100 mg/m² cisplatin administered on Day 1 of each cycle. In some aspects, vinorelbine is administered intravenously over about 5 minutes to about 10 minutes and cisplatin is administered intravenously over about 60 minutes.

In some aspects, the CCRT comprises thoracic radiotherapy and/or volumetric-modulated arc therapy (VMAT), intensity-modulated radiation therapy (IMRT), or 3-dimensional conformal radiation therapy (3DRT).

In some aspects, the radiotherapy begins on Day 1 of the second and third cycles and comprises about 30 to about 33 daily fractions of 2 Gy on a schedule of about 5 days on and 2 days off over about 6 weeks to about 7 weeks.

### II.C LAG-3 Antagonists

A LAG-3 antagonist for use in the methods of the disclosure includes, but is not limited to, LAG-3 binding agents and soluble LAG-3 polypeptides. LAG-3 binding agents include antibodies that specifically bind to LAG-3 (i.e., an “anti-LAG-3 antibody”). The term “LAG-3 antagonist” as used herein is interchangeable with the term “LAG-3 inhibitor.”

In some aspects, the LAG-3 antagonist comprises an anti-LAG-3 antibody.

Antibodies that bind to LAG-3 have been disclosed, for example, in Int'l Publ. No. WO/2015/042246 and U.S. Publ. Nos. 2014/0093511 and 2011/0150892, each of which is incorporated by reference herein in its entirety.

An exemplary LAG-3 antibody useful in the present disclosure comprises 25F7 (described in U.S. Publ. No. 2011/0150892). An additional exemplary LAG-3 antibody useful in the present disclosure comprises BMS-986016 (relatlimab). In some aspects, an anti-LAG-3 antibody useful in the present disclosure cross-competes with 25F7 or BMS-986016. In some aspects, an anti-LAG-3 antibody useful in the present disclosure binds to the same epitope as 25F7 or BMS-986016. In some aspects, an anti-LAG-3 antibody comprises six CDRs of 25F7 or BMS-986016.

Other art-recognized anti-LAG-3 antibodies that can be used in the methods of the disclosure include IMP731 (H5L7BW) described in US 2011/007023, MK-4280 (28G-10, favezelimab) described in WO2016/028672 and U.S. Publication No. 2020/0055938, REGN3767 (fianlimab) described in Burova E, et al., J. Immunother. Cancer (2016); 4 (Supp. 1): P195 and U.S. Pat. No. 10,358,495, humanized BAP050 described in WO2017/019894, GSK2831781, IMP-701 (LAG525; ieramilimab) described in U.S. Pat. No. 10,711,060 and U.S. Publ. No. 2020/0172617, aLAG3 (0414), aLAG3 (0416), Sym022, TSR-033, TSR-075, XmAb841 (previously XmAb22841), MGD013 (tebotelimab), BI754111, FS118, P 13B02-30, AVA-017, AGEN1746, RO7247669, INCAGN02385, IBI-110, EMB-02, IBI-323, LBL-007, and ABL501. These and other anti-LAG-3 antibodies useful in the claimed invention can be found in, for example: U.S. Pat. No. 10,188,730, WO 2016/028672, WO 2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO2018/185046, WO2018/185043, WO2018/217940, WO19/011306, WO2018/208868, WO2014/140180, WO2018/201096, WO2018/204374, and WO2019/018730. The contents of each of these references are incorporated by reference in their entirety.

Anti-LAG-3 antibodies that can be used in the methods of the disclosure also include isolated antibodies that bind specifically to human LAG-3 and cross-compete for binding to human LAG-3 with any anti-LAG-3 antibody disclosed herein, e.g., relatlimab. In some aspects, the anti-LAG-3 antibody binds the same epitope as any of the anti-LAG-3 antibodies described herein, e.g., relatlimab.

In some aspects, the antibodies that cross-compete for binding to human LAG-3 with, or bind to the same epitope region as, any anti-LAG-3 antibody disclosed herein, e.g., relatlimab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-LAG-3 antibodies that can be used in the methods of the disclosure also include antigen-binding portions of any of the above full-length antibodies.

In some aspects, the anti-LAG-3 antibody comprises a full-length antibody.

In some aspects, the anti-LAG-3 antibody comprises a monoclonal, human, humanized, chimeric, or multispecific antibody. In some aspects, the multispecific antibody comprises a DART, a DVD-Ig, or bispecific antibody.

In some aspects, the anti-LAG-3 antibody comprises a F(ab′)₂ fragment, a Fab′ fragment, a Fab fragment, a Fv fragment, a scFv fragment, a dsFv fragment, a dAb fragment, or a single chain binding polypeptide.

In some aspects, the anti-LAG-3 antibody comprises BMS-986016 (relatlimab), IMP731 (H5L7BW), MK4280 (28G-10, favezelimab), REGN3767 (fianlimab), GSK2831781, humanized BAP050, IMP-701 (LAG525, ieramilimab), aLAG3 (0414), aLAG3 (0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGN02385, IBI-110, EMB-02, IBI-323, LBL-007, ABL501, or an antigen binding portion thereof.

In some aspects, the anti-LAG-3 antibody is formulated for intravenous administration.

In some aspects, the anti-LAG-3 antibody is administered intravenously for about 30 minutes.

In some aspects, the anti-LAG-3 antibody comprises relatlimab.

In some aspects, the methods of the disclosure comprise an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4.

In some aspects, the methods of the disclosure comprise an anti-LAG-3 antibody comprising: (a) a heavy chain variable region CDR1 comprising the sequence set forth in SEQ ID NO:5; (b) a heavy chain variable region CDR2 comprising the sequence set forth in SEQ ID NO:6; (c) a heavy chain variable region CDR3 comprising the sequence set forth in SEQ ID NO:7; (d) a light chain variable region CDR1 comprising the sequence set forth in SEQ ID NO:8; (e) a light chain variable region CDR2 comprising the sequence set forth in SEQ ID NO:9; and (f) a light chain variable region CDR3 comprising the sequence set forth in SEQ ID NO:10.

In some aspects, the methods of the disclosure comprise an anti-LAG-3 antibody comprising heavy and light chain variable regions comprising the sequences set forth in SEQ ID NOs: 3 and 4, respectively.

In some aspects, the methods of the disclosure comprise an anti-LAG-3 antibody comprising heavy and light chains comprising the sequences set forth in SEQ ID NOs: 1 and 2, respectively.

In some aspects, the methods of the disclosure comprise an anti-LAG-3 antibody comprising heavy and light chains comprising the sequences set forth in SEQ ID NOs: 21 and 2, respectively.

In some aspects, the anti-LAG-3 antibody comprises MGD013 (tebotelimab), which is a bispecific PD-1×LAG-3 DART. In some aspects, tebotelimab is administered intravenously at a dose of about 300 mg or about 600 mg once about every 2 or 3 weeks. In some aspects, tebotelimab is administered intravenously at a dose of about 300 mg once about every 2 weeks. In some aspects, tebotelimab is administered intravenously at a dose of about 600 mg once about every 3 weeks.

In some aspects, the anti-LAG-3 antibody comprises REGN3767 (fianlimab). In some aspects, fianlimab is administered intravenously at a dose of about 1 mg/kg, about 3 mg/kg, about 10 mg/kg, or about 20 mg/kg once about every 3 weeks. In some aspects, fianlimab is administered intravenously at a dose of about 1600 mg once about every 3 weeks.

In some aspects, the methods of the disclosure comprise an anti-LAG-3 antibody comprising CDR1, CDR2 and CDR3 domains of the heavy chain variable region having the sequence set forth in SEQ ID NO:25, and CDR1, CDR2 and CDR3 domains of the light chain variable region having the sequence set forth in SEQ ID NO:26.

In some aspects, the methods of the disclosure comprise an anti-LAG-3 antibody comprising: (a) a heavy chain variable region CDR1 comprising the sequence set forth in SEQ ID NO:27; (b) a heavy chain variable region CDR2 comprising the sequence set forth in SEQ ID NO:28; (c) a heavy chain variable region CDR3 comprising the sequence set forth in SEQ ID NO:29; (d) a light chain variable region CDR1 comprising the sequence set forth in SEQ ID NO:30; (e) a light chain variable region CDR2 comprising the sequence set forth in SEQ ID NO:31; and (f) a light chain variable region CDR3 comprising the sequence set forth in SEQ ID NO:32.

In some aspects, the methods of the disclosure comprise an anti-LAG-3 antibody comprising heavy and light chain variable regions comprising the sequences set forth in SEQ ID NOs: 25 and 26, respectively.

In some aspects, the methods of the disclosure comprise an anti-LAG-3 antibody comprising heavy and light chains comprising the sequences as set forth in SEQ ID NOs: 23 and 24, respectively.

In some aspects, the anti-LAG-3 antibody comprises LAG525 (ieramilimab). In some aspects, ieramilimab is administered intravenously at a dose of about 300 mg, about 400 mg, about 500 mg, about 600 mg, about 700 mg, about 800 mg, about 900 mg, about 1000 mg, about 1100 mg, about 1200 mg, or about 1300 mg once about every 2, 3, or 4 weeks.

In some aspects, the methods of the disclosure comprise an anti-LAG-3 antibody comprising CDR1, CDR2 and CDR3 domains of the heavy chain variable region having the sequence set forth in SEQ ID NO:47, and CDR1, CDR2 and CDR3 domains of the light chain variable region having the sequence set forth in SEQ ID NO:49.

In some aspects, the methods of the disclosure comprise an anti-LAG-3 antibody comprising CDR1, CDR2 and CDR3 domains of the heavy chain variable region having the sequence set forth in SEQ ID NO:48, and CDR1, CDR2 and CDR3 domains of the light chain variable region having the sequence set forth in SEQ ID NO:50.

In some aspects, the methods of the disclosure comprise an anti-LAG-3 antibody comprising: (a) a heavy chain variable region CDR1 comprising the sequence set forth in SEQ ID NO:51; (b) a heavy chain variable region CDR2 comprising the sequence set forth in SEQ ID NO:52; (c) a heavy chain variable region CDR3 comprising the sequence set forth in SEQ ID NO:53; (d) a light chain variable region CDR1 comprising the sequence set forth in SEQ ID NO:54; (e) a light chain variable region CDR2 comprising the sequence set forth in SEQ ID NO:55; and (f) a light chain variable region CDR3 comprising the sequence set forth in SEQ ID NO:56.

In some aspects, the methods of the disclosure comprise an anti-LAG-3 antibody comprising heavy and light chain variable regions comprising the sequences set forth in SEQ ID NOs: 47 and 49, respectively.

In some aspects, the methods of the disclosure comprise an anti-LAG-3 antibody comprising heavy and light chain variable regions comprising the sequences set forth in SEQ ID NOs: 48 and 50, respectively.

In some aspects, the methods of the disclosure comprise an anti-LAG-3 antibody comprising heavy and light chains comprising the sequences as set forth in SEQ ID NOs: 43 and 45, respectively.

In some aspects, the methods of the disclosure comprise an anti-LAG-3 antibody comprising heavy and light chains comprising the sequences as set forth in SEQ ID NOs: 44 and 46, respectively.

In some aspects, the anti-LAG-3 antibody comprises MK4280 (favezelimab). In some aspects, favezelimab is administered intravenously at a dose of about 7 mg, about 21 mg, about 70 mg, about 210 mg, about 700 mg, or about 800 mg once about every 3 weeks or once about every 6 weeks. In some aspects, favezelimab is administered intravenously at a dose of about 200 mg once about every 3 weeks. In some aspects, favezelimab is administered intravenously at a dose of about 800 mg once about every 6 weeks. In some aspects, favezelimab is administered intravenously at a dose of about 800 mg on Day 1, then once about every 3 weeks. In some aspects, favezelimab is administered for up to 35 cycles. In some aspects, favezelimab is administered intravenously at a dose of about 800 mg for about 30 minutes on Day 1 of a three-week cycle for up to 35 cycles.

In some aspects, the methods of the disclosure comprise an anti-LAG-3 antibody comprising CDR1, CDR2 and CDR3 domains of the heavy chain variable region having the sequence set forth in SEQ ID NO:69, and CDR1, CDR2 and CDR3 domains of the light chain variable region having the sequence set forth in SEQ ID NO:70.

In some aspects, the methods of the disclosure comprise an anti-LAG-3 antibody comprising: (a) a heavy chain variable region CDR1 comprising the sequence set forth in SEQ ID NO:71; (b) a heavy chain variable region CDR2 comprising the sequence set forth in SEQ ID NO:72; (c) a heavy chain variable region CDR3 comprising the sequence set forth in SEQ ID NO:73; (d) a light chain variable region CDR1 comprising the sequence set forth in SEQ ID NO:74; (e) a light chain variable region CDR2 comprising the sequence set forth in SEQ ID NO:75; and (f) a light chain variable region CDR3 comprising the sequence set forth in SEQ ID NO:76.

In some aspects, the methods of the disclosure comprise an anti-LAG-3 antibody comprising heavy and light chain variable regions comprising the sequences set forth in SEQ ID NOs: 69 and 70, respectively.

In some aspects, the methods of the disclosure comprise an anti-LAG-3 antibody comprising heavy and light chains comprising the sequences as set forth in SEQ ID NOs: 67 and 68, respectively.

In some aspects, an anti-LAG-3 antibody is used to determine LAG-3 expression. In some aspects, an anti-LAG-3 antibody is selected for its ability to bind to LAG-3 in formalin-fixed, paraffin-embedded (FFPE) tissue specimens. In some aspects, an anti-LAG-3 antibody is capable of binding to LAG-3 in frozen tissues. In some aspects, an anti-LAG-3 antibody is capable of distinguishing membrane bound, cytoplasmic, and/or soluble forms of LAG-3.

In some aspects, an anti-LAG-3 antibody useful for assaying, detecting, and/or quantifying LAG-3 expression in accordance with the methods disclosed herein is the 17B4 mouse IgG1 anti-human LAG-3 monoclonal antibody. See, e.g., Matsuzaki, J et al., PNAS (2010); 107:7875.

In some aspects, the LAG-3 antagonist comprises a soluble LAG-3 polypeptide. In some aspects, the soluble LAG-3 polypeptide is a fusion polypeptide, e.g., a fusion protein comprising the extracellular portion of LAG-3. In some aspects, the soluble LAG-3 polypeptide is a LAG-3-Fc fusion polypeptide capable of binding to MHC Class II. In some aspects, the soluble LAG-3 polypeptide comprises a ligand binding fragment of the LAG-3 extracellular domain. In some aspects, the ligand binding fragment of the LAG-3 extracellular domain comprises an amino acid sequence with at least about 90%, at least about 95%, at least about 98%, at least about 99%, or about 100% sequence identity to SEQ ID NO: 22. In some aspects, the soluble LAG-3 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble LAG-3 polypeptide is IMP321 (eftilagimod alpha). See, e.g., Brignone C, et al., J. Immunol. (2007); 179:4202-4211 and WO2009/044273. In some aspects, eftilagimod alpha is administered at a dose of about 30 mg. In some aspects, eftilagimod alpha is administered subcutaneously at a dose of about 30 mg once about every 2 weeks.

In some aspects, the LAG-3 antagonist is administered at a flat dose.

In some aspects, the LAG-3 antagonist is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the LAG-3 antagonist is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the LAG-3 antagonist is administered at a weight-based dose.

In some aspects, the LAG-3 antagonist is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the LAG-3 antagonist is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the PD-1 pathway inhibitor administered with the CCRT, the CCRT, the PD-1 pathway inhibitor administered with the LAG-3 antagonist, and/or the LAG-3 antagonist are formulated for intravenous administration.

In some aspects, the PD-1 pathway inhibitor administered with the LAG-3 antagonist is formulated separately from the LAG-3 antagonist. In some aspects, the PD-1 pathway inhibitor is administered before the LAG-3 antagonist. In some aspects, the LAG-3 antagonist is administered before the PD-1 pathway inhibitor. In some aspects, the PD-1 pathway inhibitor and the LAG-3 antagonist are administered concurrently.

In some aspects, the PD-1 pathway inhibitor administered with the LAG-3 antagonist is formulated together with the LAG-3 antagonist (e.g., a fixed dose combination comprising the PD-1 pathway inhibitor and the LAG-3 antagonist).

In some aspects, the PD-1 pathway inhibitor is administered with the LAG-3 antagonist about once every four weeks. In some aspects, the PD-1 pathway inhibitor is administered with the LAG-3 antagonist as a maintenance therapy. In some aspects, the maintenance therapy is administered for up to about 1 year.

In some aspects, the PD-1 antagonist administered with the LAG-3 antagonist in the methods of the disclosure comprise nivolumab and relatlimab.

In some aspects, the PD-1 antagonist administered with the LAG-3 antagonist in the methods of the disclosure comprise: (a) an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14; and (b) an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4.

In some aspects, the PD-1 antagonist administered with the LAG-3 antagonist in the methods of the disclosure comprise: (a) an anti-PD-1 antibody comprising a heavy chain variable region CDR1, CDR2, and CDR3 comprising the sequence set forth in SEQ ID NO: 15, SEQ ID NO:16, and SEQ ID NO:17, respectively, and a light chain variable region CDR1, CDR2, and CDR3 comprising the sequence set forth in SEQ ID NO:18, SEQ ID NO: 19, and SEQ ID NO:20, respectively; and (b) an anti-LAG-3 antibody comprising a heavy chain variable region CDR1, CDR2, and CDR3 comprising the sequence set forth in SEQ ID NO:5, SEQ ID NO:6, and SEQ ID NO:7, respectively, and a light chain variable region CDR1, CDR2, and CDR3 comprising the sequence set forth in SEQ ID NO:8, SEQ ID NO: 9, and SEQ ID NO:10, respectively.

In some aspects, the PD-1 antagonist administered with the LAG-3 antagonist in the methods of the disclosure comprise: (a) an anti-PD-1 antibody comprising heavy and light chain variable regions comprising the sequences set forth in SEQ ID NOs: 13 and 14, respectively; and (b) an anti-LAG-3 antibody comprising heavy and light chain variable regions comprising the sequences set forth in SEQ ID NOs: 3 and 4, respectively.

In some aspects, the PD-1 antagonist administered with the LAG-3 antagonist in the methods of the disclosure comprise: (a) an anti-PD-1 antibody comprises heavy and light chains comprising the sequences as set forth in SEQ ID NOs: 11 and 12, respectively; and (b) an anti-LAG-3 antibody comprising heavy and light chains comprising the sequences set forth in SEQ ID NOs: 1 and 2, respectively.

In some aspects, the PD-1 antagonist administered with the LAG-3 antagonist in the methods of the disclosure comprise: (a) an anti-PD-1 antibody comprises heavy and light chains comprising the sequences as set forth in SEQ ID NOs: 11 and 12, respectively; and (b) an anti-LAG-3 antibody comprising heavy and light chains comprising the sequences set forth in SEQ ID NOs: 21 and 2, respectively.

In some aspects, the PD-1 antagonist administered with the LAG-3 antagonist in the methods of the disclosure comprise pembrolizumab and favezelimab.

In some aspects, the PD-1 antagonist administered with the LAG-3 antagonist in the methods of the disclosure comprise: (a) an anti-PD-1 antibody comprising CDR1, CDR2 and CDR3 domains of the heavy chain variable region having the sequence set forth in SEQ ID NO:79, and CDR1, CDR2 and CDR3 domains of the light chain variable region having the sequence set forth in SEQ ID NO:80; and (b) an anti-LAG-3 antibody comprising CDR1, CDR2 and CDR3 domains of the heavy chain variable region having the sequence set forth in SEQ ID NO:69, and CDR1, CDR2 and CDR3 domains of the light chain variable region having the sequence set forth in SEQ ID NO:70.

In some aspects, the PD-1 antagonist administered with the LAG-3 antagonist in the methods of the disclosure comprise: (a) an anti-PD-1 antibody comprising a heavy chain variable region CDR1, CDR2, and CDR3 comprising the sequence set forth in SEQ ID NO: 81, SEQ ID NO:82, and SEQ ID NO:83, respectively, and a light chain variable region CDR1, CDR2, and CDR3 comprising the sequence set forth in SEQ ID NO:84, SEQ ID NO: 85, and SEQ ID NO:86, respectively; and (b) an anti-LAG-3 antibody comprising a heavy chain variable region CDR1, CDR2, and CDR3 comprising the sequence set forth in SEQ ID NO:71, SEQ ID NO:72, and SEQ ID NO:73, respectively, and a light chain variable region CDR1, CDR2, and CDR3 comprising the sequence set forth in SEQ ID NO: 74, SEQ ID NO:75, and SEQ ID NO: 76, respectively.

In some aspects, the PD-1 antagonist administered with the LAG-3 antagonist in the methods of the disclosure comprise: (a) an anti-PD-1 antibody comprising heavy and light chain variable regions comprising the sequences set forth in SEQ ID NOs: 79 and 80, respectively; and (b) an anti-LAG-3 antibody comprising heavy and light chain variable regions comprising the sequences set forth in SEQ ID NOs: 69 and 70, respectively.

In some aspects, the PD-1 antagonist administered with the LAG-3 antagonist in the methods of the disclosure comprise: (a) an anti-PD-1 antibody comprises heavy and light chains comprising the sequences as set forth in SEQ ID NOs: 77 and 78, respectively; and (b) an anti-LAG-3 antibody comprising heavy and light chains comprising the sequences set forth in SEQ ID NOs: 67 and 68, respectively.

In some aspects, the PD-1 antagonist administered with the LAG-3 antagonist in the methods of the disclosure comprise cemiplimab and fianlimab.

In some aspects, the PD-1 antagonist administered with the LAG-3 antagonist in the methods of the disclosure comprise: (a) an anti-PD-1 antibody comprising CDR1, CDR2 and CDR3 domains of the heavy chain variable region having the sequence set forth in SEQ ID NO:35, and CDR1, CDR2 and CDR3 domains of the light chain variable region having the sequence set forth in SEQ ID NO:36; and (b) an anti-LAG-3 antibody comprising CDR1, CDR2 and CDR3 domains of the heavy chain variable region having the sequence set forth in SEQ ID NO:25, and CDR1, CDR2 and CDR3 domains of the light chain variable region having the sequence set forth in SEQ ID NO:26.

In some aspects, the PD-1 antagonist administered with the LAG-3 antagonist in the methods of the disclosure comprise: (a) an anti-PD-1 antibody comprising a heavy chain variable region CDR1, CDR2, and CDR3 comprising the sequence set forth in SEQ ID NO: 37, SEQ ID NO:38, and SEQ ID N0.39, respectively, and a light chain variable region CDR1, CDR2, and CDR3 comprising the sequence set forth in SEQ ID NO:40, SEQ ID NO: 41, and SEQ ID NO:42, respectively; and (b) an anti-LAG-3 antibody comprising a heavy chain variable region CDR1, CDR2, and CDR3 comprising the sequence set forth in SEQ ID NO:27. SEQ ID NO:28, and SEQ ID NO:29, respectively, and a light chain variable region CDR1, CDR2, and CDR3 comprising the sequence set forth in SEQ ID NO: 30, SEQ ID NO:31, and SEQ ID NO:32, respectively.

In some aspects, the PD-1 antagonist administered with the LAG-3 antagonist in the methods of the disclosure comprise: (a) an anti-PD-1 antibody comprising heavy and light chain variable regions comprising the sequences set forth in SEQ ID NOs: 35 and 36, respectively; and (b) an anti-LAG-3 antibody comprising heavy and light chain variable regions comprising the sequences set forth in SEQ ID NOs: 25 and 26, respectively.

In some aspects, the PD-1 antagonist administered with the LAG-3 antagonist in the methods of the disclosure comprise: (a) an anti-PD-1 antibody comprises heavy and light chains comprising the sequences as set forth in SEQ ID NOs: 33 and 34, respectively; and (b) an anti-LAG-3 antibody comprising heavy and light chains comprising the sequences set forth in SEQ ID NOs: 23 and 24, respectively

In some aspects, the PD-1 antagonist administered with the LAG-3 antagonist in the methods of the disclosure comprise spartalizumab and ieramilimab.

In some aspects, the PD-1 antagonist administered with the LAG-3 antagonist in the methods of the disclosure comprise: (a) an anti-PD-1 antibody comprising CDR1, CDR2 and CDR3 domains of the heavy chain variable region having the sequence set forth in SEQ ID NO:59, and CDR1, CDR2 and CDR3 domains of the light chain variable region having the sequence set forth in SEQ ID NO:60; and (b) an anti-LAG-3 antibody comprising CDR1, CDR2 and CDR3 domains of the heavy chain variable region having the sequence set forth in SEQ ID NO:47, and CDR1, CDR2 and CDR3 domains of the light chain variable region having the sequence set forth in SEQ ID NO:49.

In some aspects, the PD-1 antagonist administered with the LAG-3 antagonist in the methods of the disclosure comprise: (a) an anti-PD-1 antibody comprising CDR1, CDR2 and CDR3 domains of the heavy chain variable region having the sequence set forth in SEQ ID NO:59, and CDR1, CDR2 and CDR3 domains of the light chain variable region having the sequence set forth in SEQ ID NO:60; and (b) an anti-LAG-3 antibody comprising CDR1, CDR2 and CDR3 domains of the heavy chain variable region having the sequence set forth in SEQ ID NO:48, and CDR1, CDR2 and CDR3 domains of the light chain variable region having the sequence set forth in SEQ ID NO:50.

In some aspects, the PD-1 antagonist administered with the LAG-3 antagonist in the methods of the disclosure comprise: (a) an anti-PD-1 antibody comprising a heavy chain variable region CDR1, CDR2, and CDR3 comprising the sequence set forth in SEQ ID NO: 61, SEQ ID NO:62, and SEQ ID NO:63, respectively, and a light chain variable region CDR1, CDR2, and CDR3 comprising the sequence set forth in SEQ ID NO:64, SEQ ID NO: 65, and SEQ ID NO:66, respectively; and (b) an anti-LAG-3 antibody comprising a heavy chain variable region CDR1, CDR2, and CDR3 comprising the sequence set forth in SEQ ID NO:51, SEQ ID NO:52, and SEQ ID NO:53, respectively, and a light chain variable region CDR1, CDR2, and CDR3 comprising the sequence set forth in SEQ ID NO: 54, SEQ ID NO:55, and SEQ ID NO:56, respectively.

In some aspects, the PD-1 antagonist administered with the LAG-3 antagonist in the methods of the disclosure comprise: (a) an anti-PD-1 antibody comprising heavy and light chain variable regions comprising the sequences set forth in SEQ ID NOs: 59 and 60, respectively; and (b) an anti-LAG-3 antibody comprising heavy and light chain variable regions comprising the sequences set forth in SEQ ID NOs: 47 and 49, respectively.

In some aspects, the PD-1 antagonist administered with the LAG-3 antagonist in the methods of the disclosure comprise: (a) an anti-PD-1 antibody comprising heavy and light chain variable regions comprising the sequences set forth in SEQ ID NOs: 59 and 60, respectively; and (b) an anti-LAG-3 antibody comprising heavy and light chain variable regions comprising the sequences set forth in SEQ ID NOs: 48 and 50, respectively.

In some aspects, the PD-1 antagonist administered with the LAG-3 antagonist in the methods of the disclosure comprise: (a) an anti-PD-1 antibody comprises heavy and light chains comprising the sequences as set forth in SEQ ID NOs: 57 and 58, respectively; and (b) an anti-LAG-3 antibody comprising heavy and light chains comprising the sequences set forth in SEQ ID NOs: 43 and 45, respectively.

In some aspects, the PD-1 antagonist administered with the LAG-3 antagonist in the methods of the disclosure comprise: (a) an anti-PD-1 antibody comprises heavy and light chains comprising the sequences as set forth in SEQ ID NOs: 57 and 58, respectively; and (b) an anti-LAG-3 antibody comprising heavy and light chains comprising the sequences set forth in SEQ ID NOs: 44 and 46, respectively.

Provided herein is a method of treating a human subject afflicted with a NSCLC that has a squamous or non-squamous histology, the method comprising: (a) administering to the subject about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, and a CCRT comprising a PDCT and radiotherapy, (b) providing the subject with a recovery period that begins upon the completion of the administration in (a), followed by (c) administering to the subject a maintenance therapy comprising about 480 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, and about 480 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4.

In some aspects, the PDCT comprises cisplatin and etoposide. In some aspects, (a) comprises three 21-day cycles, wherein the anti-PD-1 antibody is administered on Day 1 of each cycle, wherein about 100 mg/m² of the etoposide is administered on Days 1, 2, and 3 of each cycle, and after the anti-PD-1 antibody on Day 1 of each cycle, and wherein about 80 mg/m² of the cisplatin is administered on Day 1 of each cycle after the etoposide. In some aspects, each of the cisplatin and the etoposide is administered intravenously over about 60 minutes.

In some aspects, the PDCT comprises carboplatin and paclitaxel. In some aspects, (a) comprises three 21-day cycles, wherein the anti-PD-1 antibody is administered on Day 1 of each cycle, wherein about 175 mg/m² or about 200 mg/m² of the paclitaxel is administered on Day 1 of the first cycle, wherein about 45 mg/m² or about 50 mg/m² of the paclitaxel is administered on Days 1, 8, and 15 of the second and third cycles, and wherein the paclitaxel is administered after the anti-PD-1 antibody on Day 1 of each cycle, and wherein the carboplatin at a target AUC of about 5 mg/mL·min or about 6 mg/mL·min is administered on Day 1 of the first cycle, wherein the carboplatin at a target AUC of about 2 mg/mL·min is administered on Days 1, 8, and 15 of the second and third cycles, and wherein the carboplatin is administered after the paclitaxel in each cycle. In some aspects, the carboplatin is administered intravenously over about 30 minutes, and the paclitaxel is administered intravenously over about 180 minutes in the first cycle and over about 60 minutes in the second and third cycles.

In some aspects, the PDCT comprises cisplatin and pemetrexed. In some aspects, (a) comprises three 21-day cycles, wherein the anti-PD-1 antibody is administered on Day 1 of each cycle, wherein about 500 mg/m² of the pemetrexed is administered on Day 1 of each cycle, after the anti-PD-1 antibody, and wherein about 75 mg/m² of the cisplatin is administered on Day 1 of each cycle after the pemetrexed. In some aspects, the cisplatin is administered intravenously over about 60 minutes and the pemetrexed is administered intravenously over about 10 minutes. In some aspects, the cisplatin is replaced with carboplatin at a target area under the concentration time-curve (AUC) of about 5 mg/mL·min if the cisplatin is not tolerated by the subject. In some aspects, the carboplatin is administered intravenously over about 30 minutes. In some aspects, the pemetrexed is replaced with etoposide if the pemetrexed is not tolerated by the subject.

In some aspects, the anti-PD-1 antibody in (a) is administered intravenously over about 30 minutes.

In some aspects, the radiotherapy is administered after the PDCT and comprises a dose of about 60 Gy to about 66 Gy.

In some aspects, the radiotherapy comprises thoracic radiotherapy and/or volumetric-modulated arc therapy (VMAT), intensity-modulated radiation therapy (IMRT), or 3-dimensional conformal radiation therapy (3DRT). In some aspects, the radiotherapy begins on Day 1 of the second and third cycles and comprises about 30 to about 33 daily fractions of 2 Gy on a schedule of about 5 days on and 2 days off over about 6 weeks to about 7 weeks.

In some aspects, the recovery period is a duration sufficient for the subject to recover from toxicities associated with the CCRT other than fatigue, esophagitis, or alopecia. In some aspects, the recovery period is from about 1 week to about 12 weeks, about 1 week to about 9 weeks, about 1 week to about 6 weeks, about 2 weeks to about 12 weeks, about 2 weeks to about 9 weeks, about 2 weeks to about 6 weeks, about 3 weeks to about 12 weeks, about 3 weeks to about 9 weeks, about 3 weeks to about 6 weeks, about 18 days from final administration of the anti-PD-1 antibody in (a) to about 12 weeks, about 18 days from final administration of the anti-PD-1 antibody in (a) to about 9 weeks, or about 18 days from final administration of the anti-PD-1 antibody in (a) to about 6 weeks.

In some aspects, the method is a first line therapy.

In some aspects, the subject has not received a prior local or systemic anticancer therapy given as primary therapy for locally advanced disease.

In some aspects, the subject has not received a prior systemic therapy for cancer, the subject has not received a prior systemic therapy for lung cancer, or the subject has not received a prior systemic therapy for advanced or metastatic lung cancer.

In some aspects, the subject is naïve to prior immuno-oncology therapy, the subject is naïve to prior immuno-oncology therapy for lung cancer, or the lung cancer is naïve to prior immuno-oncology therapy.

In some aspects, the method is a second line therapy.

In some aspects, the method is a third line therapy.

In some aspects, the subject has progressed on a prior therapy.

In some aspects, the NSCLC is recurrent following multi-modal therapy for locally advanced NSCLC.

In some aspects, the NSCLC is unresectable, advanced, recurrent, and/or metastatic. In some aspects, the NSCLC has a squamous or non-squamous histology.

In some aspects, the NSCLC comprises locally advanced Stage IIIA, IIIB, or IIIC NSCLC.

In some aspects, the subject is free of progressive NSCLC during the CCRT or the recovery period.

In some aspects, the anti-PD-1 and the anti-LAG-3 antibodies in (b) are administered once about every 4 weeks.

In some aspects, the anti-PD-1 and the anti-LAG-3 antibodies in (b) are formulated separately. In some aspects, the anti-PD-1 antibody in (b) is administered before the anti-LAG-3 antibody. In some aspects, the anti-LAG-3 antibody is administered before the anti-PD-1 antibody in (b). In some aspects, the anti-PD-1 and the anti-LAG-3 antibodies in (b) are administered concurrently.

In some aspects, the anti-PD-1 and the anti-LAG-3 antibodies in (b) are formulated together.

In some aspects, the anti-PD-1 and the anti-LAG-3 antibodies in (b) are administered intravenously over about 30 minutes.

In some aspects, the anti-PD-1 antibody in (a) and/or (b) comprises a full-length antibody.

In some aspects, the anti-PD-1 antibody in (a) and/or (b) comprises a monoclonal, human, humanized, chimeric, or multispecific antibody. In some aspects, the multispecific antibody comprises a DART, a DVD-Ig, or bispecific antibody.

In some aspects, the anti-PD-1 antibody in (a) and/or (b) comprises a F(ab′)₂ fragment, a Fab′ fragment, a Fab fragment, a Fv fragment, a scFv fragment, a dsFv fragment, a dAb fragment, or a single chain binding polypeptide

In some aspects, the anti-PD-1 antibody in (a) and/or (b) comprises: (a) a heavy chain variable region CDR1 comprising the sequence set forth in SEQ ID NO:15; (b) a heavy chain variable region CDR2 comprising the sequence set forth in SEQ ID NO:16; (c) a heavy chain variable region CDR3 comprising the sequence set forth in SEQ ID NO: 17; (d) a light chain variable region CDR1 comprising the sequence set forth in SEQ ID NO: 18; (e) a light chain variable region CDR2 comprising the sequence set forth in SEQ ID NO: 19; and (f) a light chain variable region CDR3 comprising the sequence set forth in SEQ ID NO:20. In some aspects, the anti-PD-1 antibody in (a) and/or (b) comprises heavy and light chain variable regions comprising the sequences set forth in SEQ ID NOs: 13 and 14, respectively. In some aspects, the anti-PD-1 antibody in (a) and/or (b) comprises heavy and light chains comprising the sequences as set forth in SEQ ID NOs: 11 and 12, respectively.

In some aspects, the anti-LAG-3 antibody comprises a full-length antibody.

In some aspects, the anti-LAG-3 antibody comprises a monoclonal, human, humanized, chimeric, or multispecific antibody. In some aspects, the multispecific antibody comprises a DART, a DVD-Ig, or bispecific antibody.

In some aspects, the anti-LAG-3 antibody comprises a F(ab′)₂ fragment, a Fab′ fragment, a Fab fragment, a Fv fragment, a scFv fragment, a dsFv fragment, a dAb fragment, or a single chain binding polypeptide.

In some aspects, the anti-LAG-3 antibody comprises: (a) a heavy chain variable region CDR1 comprising the sequence set forth in SEQ ID NO:5; (b) a heavy chain variable region CDR2 comprising the sequence set forth in SEQ ID NO:6; (c) a heavy chain variable region CDR3 comprising the sequence set forth in SEQ ID NO:7; (d) a light chain variable region CDR1 comprising the sequence set forth in SEQ ID NO:8; (e) a light chain variable region CDR2 comprising the sequence set forth in SEQ ID NO:9; and (f) a light chain variable region CDR3 comprising the sequence set forth in SEQ ID NO:10. In some aspects, the anti-LAG-3 antibody comprises heavy and light chain variable regions comprising the sequences set forth in SEQ ID NOs: 3 and 4, respectively. In some aspects, the anti-LAG-3 antibody comprises heavy and light chains comprising the sequences set forth in SEQ ID NOs: 1 and 2, respectively. In some aspects, the anti-LAG-3 antibody comprises heavy and light chains comprising the sequences set forth in SEQ ID NOs: 21 and 2, respectively.

II.D LAG-3 and/or PD-L1 Expression

In some aspects, one or more immune cells in tumor tissue from the subject in the methods disclosed herein express LAG-3 (i.e., tumor tissue from the patient is LAG-3 positive) and/or one or more nucleated cells in tumor tissue from the subject express LAG-3 (i.e., tumor tissue from the patient is LAG-3 positive) and/or one or more tumor cells in tumor tissue from the subject express PD-L1 (i.e., tumor tissue from the patient is PD-L1 positive).

In some aspects, one or more immune cells in tumor tissue from the subject express LAG-3. In some aspects, at least about 1%, at least about 2%, at least about 3%, at least about 4%, at least about 5%, at least about 7%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3. In some aspects, at least about 1% of the immune cells express LAG-3. In some aspects, greater than about 1% of the immune cells express LAG-3. In some aspects, at least about 5% of the immune cells express LAG-3. In some aspects, the immune cells comprise tumor-infiltrating lymphocytes. In some aspects, the tumor-infiltrating lymphocytes comprise CD8⁺ cells.

In some aspects, one or more nucleated cells in tumor tissue from the subject express LAG-3. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the nucleated cells express LAG-3. In some aspects, at least about 1% of the nucleated cells express LAG-3. In some aspects, greater than about 1% of the nucleated cells express LAG-3. In some aspects, at least about 5% of the nucleated cells express LAG-3.

In some aspects, one or more tumor cells in tumor tissue from the subject express PD-L1. In some aspects, at least about 1%, at least about 2%, at least about 3%, at least about 4%, at least about 5%, at least about 7%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1. In some aspects, at least about 1% of the tumor cells express PD-L1. In some aspects, at least about 1% of the tumor cells express PD-L1. In some aspects, greater than about 1% of the tumor cells express PD-L1. In some aspects, at least about 5% of the tumor cells express PD-L1.

In some aspects, one or more nucleated cells in tumor tissue from the subject express PD-L1. In some aspects, at least about 1%, at least about 2%, at least about 3%, at least about 4%, at least about 5%, at least about 7%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the nucleated cells express PD-L1. In some aspects, at least about 1% of the nucleated cells express PD-L1. In some aspects, at least about 1% of the nucleated cells express PD-L1. In some aspects, greater than about 1% of the nucleated cells express PD-L1. In some aspects, at least about 5% of the nucleated cells express PD-L1.

In some aspects, any of the values of “at least about X %” is “≥X %”).

In some aspects, tumor tissue from the patient is LAG-3 negative. In some aspects, the tumor tissue is LAG-3 negative when less than about 1% of the immune cells express LAG-3. In some aspects, the tumor tissue is LAG-3 negative when less than about 1% of the nucleated cells express LAG-3.

In some aspects, tumor tissue from the patient is PD-1 negative. In some aspects, the tumor tissue is PD-1 negative when less than about 1% of the immune cells express PD-1. In some aspects, the tumor tissue is PD-1 negative when less than about 1% of the nucleated cells express PD-1.

In some aspects, tumor tissue from the patient is PD-L1 negative. In some aspects, the tumor tissue is PD-L1 negative when less than about 1% of the tumor cells express PD-L1. In some aspects, the tumor tissue is PD-L1 negative when less than about 1% of the nucleated cells express PD-L1.

In some aspects, LAG-3, PD-1, and/or PD-L1 expression in the subject's tumor tissue is determined from a test tissue sample. In some aspects, a test tissue sample includes, but is not limited to, any clinically relevant tissue sample, such as a tumor biopsy, a core biopsy, an incisional biopsy, an excisional biopsy, a surgical specimen, a fine needle aspirate, or a sample of bodily fluid, such as blood, plasma, serum, lymph, ascites fluid, cystic fluid, or urine. In some aspects, the test tissue sample is from a primary tumor. In some aspects, the test tissue sample is from a metastasis. In some aspects, test tissue samples are from multiple time points, for example, before treatment, during treatment, and/or after treatment. In some aspects, test tissue samples are from different locations in the subject, for example, from a primary tumor and from a metastasis.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen tissue sample. In some aspects, the test tissue sample is a fresh frozen (FF) tissue (e.g., tumor) sample. In some aspects, the test tissue sample is a cell isolated from a fluid. In some aspects, the test tissue sample comprises circulating tumor cells (CTCs). In some aspects, the test tissue sample comprises tumor-infiltrating lymphocytes (TILs). In some aspects, the test tissue sample comprises tumor cells and tumor-infiltrating lymphocytes (TILs). In some aspects, the test tissue sample comprises circulating lymphocytes. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×10⁶ cells or more. In some aspects, the sample size is about 1 cell to about 1×10⁵ cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell.

In some aspects, LAG-3, PD-1, and/or PD-L1 expression is assessed by performing an assay to detect the presence of LAG-3, PD-1, and/or PD-L1 RNA, respectively. In some aspects, the presence of LAG-3, PD-1, and/or PD-L1 RNA is detected by RT-PCR, in situ hybridization or RNase protection.

In some aspects, LAG-3, PD-1, and/or PD-L1 expression is assessed by performing an assay to detect the presence of LAG-3, PD-1, and/or PD-L1 polypeptide, respectively. In some aspects, the presence of LAG-3, PD-1, and/or PD-L1 polypeptide is detected by immunohistochemistry (IHC), enzyme-linked immunosorbent assay (ELISA), in vivo imaging, or flow cytometry.

### II.E. Additional Therapeutic Agents

The methods disclosed herein can comprise an additional therapeutic agent and/or anti-cancer therapy, which can comprise any known therapeutic agent or anti-cancer therapy, including a standard of care in the art for the treatment of a subject afflicted with lung cancer. In some aspects, the therapeutic agent and/or therapy is described by the NCCN Guidelines® for treatment of NSCLC. See, e.g., therapeutic agents and therapies described at: https://www.cancertherapyadvisor.com/home/cancer-topics/lung-cancer/lung-cancer-treatment-regimens-landing-page/non-small-cell-lung-cancer-treatment-regimens/, last accessed Dec. 20, 2022.

### II.E.1. Therapeutic Agents

In some aspects, the additional therapeutic agent comprises an anti-cancer agent. In some aspects, the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

In some aspects, the tyrosine kinase inhibitor comprises sorafenib (e.g., sorafenib tosylate, also known as NEXAVAR®), lenvatinib (e.g., lenvatinib mesylate, also known as LENVIMA®), regorafenib (e.g., STIVARGA®), cabozantinib (e.g., cabozantinib S-malate, also known as CABOMETYX®), sunitinib (e.g., sunitinib malate, also known as SUTENT®), brivanib, linifanib, pemigatinib (also known as PEMAZYRE™), everolimus (also known as AFINITOR® or ZORTRESS®), gefitinib (IRESSA®, a small-molecule TKI of EGFR), imatinib (e.g., imatinib mesylate), lapatinib (e.g., lapatinib ditosylate, also known as TYKERB®), nilotinib (e.g., nilotinib hydrochloride, also known as TASIGNA®), pazopanib (e.g., pazopanib hydrochloride, also known as VOTRIENT®), temsirolimus (also known as TORISEL®), erlotinib (e.g., erlotinib hydrochloride, also known as TARCEVA®, a small-molecule TKI of EGFR), afatinib (GILOTRIF®, a small-molecule TKI of EGFR), dacomitinib (VIZIMPRO®, a small-molecule TKI of EGFR), osimeritinb (TAGRISSO®, a small-molecule TKI of EGFR), alectinib (ALECENSA®, a small-molecule TKI of ALK), ceritinib (ZYKADIA®, a small-molecule TKI of ALK and ROS-1), brigatinib (ALUNBRIG®, a small-molecule TKI of ALK), crizotinib (XALKORI®, a small-molecule TKI of ALK and ROS-1), lorlatinib (LORBRENA®, a small-molecule TKI of ALK and ROS-1), entrectinib (ROZLYTREK®, a small-molecule TKI of ROS-1 and NTRK), dabrafenib (TAFINLAR®, a small-molecule TKI of BRAF) trametinib (MEKINIST®, a small-molecule TKI of BRAF), vemurafenib (ZELBORAF®, a small-molecule TKI of BRAF), larotrectinib (ROZLYTREK®, a small-molecule TKI of NTRK), or any combination thereof.

In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGFR, or any combination thereof. In some aspects, the anti-angiogenesis agent comprises bevacizumab (also known as AVASTIN®), ramucirumab (also known as CYRAMZA®), aflibercept (also known as EYLEA® or ZALTRAP®), tanibirumab, olaratumab (also known as LARTRUVO™), nesvacumab, AMG780, MEDI3617, vanucizumab, rilotumumab, ficlatuzumab, TAK-701, onartuzumab, emibetuzumab, or any combination thereof.

In some aspects, the anti-angiogenesis agent is bevacizumab. In some aspects, bevacizumab is administered at a dose of about 15 mg/kg. In some aspects, bevacizumab is administered at a dose of about 15 mg/kg on Day 1 of a three-week cycle.

In some aspects, the checkpoint stimulator comprises an agonist of B7-1, B7-2, CD28, 4-1BB (CD137), 4-1BBL, GITR, inducible T cell co-stimulator (ICOS), ICOS-L, OX40, OX40L, CD70, CD27, CD40, death receptor 3 (DR3), CD28H, or any combination thereof.

In some aspects, the chemotherapeutic agent comprises an alkylating agent, an antimetabolite, an antineoplastic antibiotic, a mitotic inhibitor, a hormone or hormone modulator, a protein tyrosine kinase inhibitor, an epidermal growth factor inhibitor, a proteasome inhibitor, other neoplastic agent, or any combination thereof.

In some aspects, the immunotherapeutic agent comprises an antibody that specifically binds to EGFR (e.g., cetuximab (ERBITUX®)), ALK, ROS-1, NTRK, BRAF, ICOS, CD137 (4-1BB), CD134 (OX40), NKG2A, CD27, CD96, GITR, Herpes Virus Entry Mediator (HVEM), PD-1, PD-L1, CTLA-4, BTLA, TIM-3, A2aR, Killer cell Lectin-like Receptor G1 (KLRG-1), Natural Killer Cell Receptor 2B4 (CD244), CD160, TIGIT, VISTA, KIR, TGFβ, IL-10, IL-8, B7-H4, Fas ligand, CSF1R, CXCR4, mesothelin, CEACAM⁻¹, CD52, HER2, MICA, MICB, or any combination thereof.

In some aspects, the platinum agent comprises cisplatin, carboplatin, oxaliplatin, satraplatin, picoplatin, nedaplatin, triplatin (e.g., triplatin tetranitrate), lipoplatin, phenanthriplatin, or any combination thereof.

In some aspects, the alkylating agent comprises altretamine, bendamustine, busulfan, carboplatin, carmustine, chlorambucil, cisplatin, cyclophosphamide, dacarbazine, ifosfamide, lomustine, mechlorethamine, melphalan, oxaliplatin, procarbazine, streptozocin, temozolomide, thiotepa, or any combination thereof.

In some aspects, the taxane comprises paclitaxel, albumin-bound paclitaxel (i.e., nab-paclitaxel), docetaxel, cabazitaxel, or any combination thereof.

In some aspects, the nucleoside analog comprises cytarabine, gemcitabine, lamivudine, entecavir, telbivudine, or any combination thereof.

In some aspects, the antimetabolite comprises capecitabine, cladribine, clofarabine, cytarabine, floxuridine, fludarabine, fluorouracil, mercaptopurine, methotrexate, pemetrexed, pentostatin, pralatrexate, thioguanine, or any combination thereof.

In some embodiments, the topoisomerase inhibitor comprises etoposide, mitoxantrone, doxorubicin, irinotecan, topotecan, camptothecin, or any combination thereof.

In some aspects, the anthracycline is doxorubicin, daunorubicin, epirubicin, idarubicin, or any combination thereof.

In some aspects, the vinca alkaloid is vinblastine, vincristine, vinorelbine, vindesine, vincaminol, vineridine, vinburnine, or any combination thereof.

### II.E.2. Checkpoint Inhibitors

In some aspects, the anti-cancer agent that is administered as an additional therapeutic agent in the methods of the disclosure is a checkpoint inhibitor.

In some aspects, the checkpoint inhibitor comprises a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM⁻¹ inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor (e.g., an indoleamine 2,3-dioxygenase 1 (IDO1) inhibitor, epacadostat (INCB24360), navoximod (GDC-0919), or linrodostat (BMS-986205), including a linrodostat salt such as, for example, linrodostat mesylate), a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM⁻¹) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

In some aspects, the checkpoint inhibitor is formulated for intravenous administration.

In some aspects, the checkpoint inhibitor is administered at a flat dose.

In some aspects, the checkpoint inhibitor is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the checkpoint inhibitor is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the checkpoint inhibitor is administered as a weight-based dose.

In some aspects, the checkpoint inhibitor is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the checkpoint inhibitor is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose of the checkpoint inhibitor is administered every one week, every two weeks, every three weeks, every four weeks, every five weeks, every six weeks, every seven weeks, every eight weeks, every nine weeks, every ten weeks, every eleven weeks, or every twelve weeks.

### II.E.3 CTLA-4 Inhibitors

In some aspects, the checkpoint inhibitor as disclosed herein comprises a CTLA-4 inhibitor. In some aspects, the CTLA-4 inhibitor comprises an anti-CTLA-4 antibody.

Anti-CTLA-4 antibodies that can be used in the methods of the disclosure bind to human CTLA-4 and disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances, or prolongs the activation of such T cells, thereby inducing, enhancing or prolonging an immune response.

Human monoclonal antibodies that bind specifically to CTLA-4 with high affinity have been disclosed in U.S. Pat. Nos. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 109 M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

Anti-CTLA-4 antibodies that can be used in the methods of the disclosure include ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2 (3): 133-39 (2007)).

In some aspects, the anti-CTLA-4 antibody binds specifically to human CTLA-4 and cross-competes for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab.

In some aspects, the antibodies that cross-compete for binding to human CTLA-4 with, or bind to the same epitope region as, any anti-CTLA-4 antibody disclosed herein, e.g., ipilimumab and/or tremelimumab, are monoclonal antibodies. For administration to human subjects, these cross-competing antibodies are chimeric antibodies, engineered antibodies, or humanized or human antibodies.

Anti-CTLA-4 antibodies that can be used in the methods of the disclosure also include antigen-binding portions of any of the above full-length antibodies.

In some aspects, the anti-CTLA-4 antibody comprises a full-length antibody. In some aspects, the anti-CTLA-4 antibody comprises a monoclonal, human, humanized, chimeric, or multispecific antibody. In some aspects, the multispecific antibody comprises a DART, a DVD-Ig, or bispecific antibody.

In some aspects, the anti-CTLA-4 antibody comprises a F(ab′)₂ fragment, a Fab′ fragment, a Fab fragment, a Fv fragment, a scFv fragment, a dsFv fragment, a dAb fragment, or a single chain binding polypeptide.

In some aspects, the anti-CTLA-4 antibody comprises ipilimumab, tremelimumab, MK-1308, AGEN-1884, or an antigen binding portion thereof.

In some aspects, the anti-CTLA-4 antibody comprises ipilimumab. Ipilimumab is a fully human, IgG1 monoclonal antibody that blocks the binding of CTLA-4 to its B7 ligands, thereby stimulating T cell activation. In some aspects, ipilimumab is administered at a dose of about 3 mg/kg once about every 3 weeks. In some aspects, ipilimumab is administered at a dose of about 10 mg/kg once about every 3 weeks. In some aspects, ipilimumab is administered at a dose of about 10 mg/kg once about every 12 weeks. In some aspects, the ipilimumab is administered for four doses. In some aspects, ipilimumab is administered on Day 1 of each cycle.

### II.E.4. Therapies for Sensitizing Mutations

In some aspects, a method of the disclosure comprises treatment of a subject with a mutation sensitive to targeted inhibitor therapy such as a sensitizing mutation in a gene such as EGFR, ALK, ROS-1, NTRK, or BRAF. Such methods can further comprise administration of a targeted inhibitor of the mutated genes, including standard of care therapies for subjects having such mutations who are afflicted with NSCLC.

In some aspects, a method of the disclosure comprises administering to a subject afflicted with advanced or metastatic NSCLC who has a sensitizing EGFR mutation afatinib (e.g., 40 mg orally once daily), erlotinib (e.g., 150 mg orally once daily), dacomitinib (e.g., 45 mg orally once daily), gefitinib (e.g., 250 mg orally once daily), or osimertinib (e.g., 80 mg orally once daily).

In some aspects, a method of the disclosure comprises administering to a subject afflicted with advanced or metastatic NSCLC who has a sensitizing EGFR mutation afatinib and cetuximab (e.g., 40 mg afatinib orally once daily on Days 1-14 and cetuximab at 500 mg/m² on Day 1 in 2-week cycles) or osimertinib (e.g., 80 mg orally once daily).

In some aspects, a method of the disclosure comprises administering to a subject afflicted with advanced or metastatic NSCLC who has a sensitizing ALK mutation (e.g., ALK rearrangement) alectinib (e.g., 600 mg orally twice daily), brigatinib (e.g., a 4 week cycle of 90 mg orally once daily on Days 1-7, 180 mg orally once daily on days 8-28 followed by 180 mg orally once daily on Days 29-56), ceritinib (e.g., 450 mg orally once daily), or crizotinib (e.g., 250 mg orally twice daily).

In some aspects, a method of the disclosure comprises administering to a subject afflicted with advanced or metastatic NSCLC who has a sensitizing ALK mutation (e.g., ALK rearrangement) lorlatinib (e.g., 100 mg orally once daily).

In some aspects, a method of the disclosure comprises administering to a subject afflicted with advanced or metastatic NSCLC who has a sensitizing ROS-1 mutation (e.g., ROS-1 rearrangement) ceritinib (e.g., 450 mg orally once daily), crizotinib (e.g., 250 mg orally twice daily), or entrectinib (e.g., 600 mg orally once daily). In some aspects, a method of the disclosure comprises administering to a subject afflicted with advanced or metastatic NSCLC who has a sensitizing ROS-1 mutation (e.g., ROS-1 rearrangement) lorlatinib (e.g., 100 mg orally once daily).

In some aspects, a method of the disclosure comprises administering to a subject afflicted with advanced or metastatic NSCLC who has a sensitizing BRAF mutation (e.g., BRAF V600E) dabrafenib (e.g., 150 mg orally twice daily), dabrafenib and trametinib (e.g., 150 mg orally twice daily and 2 mg orally once daily trametinib), or vemurafenib (e.g., 960 mg orally once daily).

In some aspects, a method of the disclosure comprises administering to a subject afflicted with advanced or metastatic NSCLC who has a sensitizing NTRK mutation (e.g., NTRK gene fusion) entrectinib (e.g., 600 mg orally once daily) or larotrectinib (e.g., 100 mg orally twice daily).

### III. Pharmaceutical Compositions

Therapeutic agents of the present disclosure can be constituted in a composition, e.g., a pharmaceutical composition containing an inhibitor, antibody, and/or agent as disclosed herein and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible.

In some aspects, the carrier for a composition containing an inhibitor, antibody, and/or agent as disclosed herein is suitable for intravenous, intramuscular, subcutaneous, parenteral, spinal or epidermal administration (e.g., by injection or infusion). In some aspects, the carrier is suitable for non-parenteral, e.g., oral, administration. In some aspects, a subcutaneous injection is based on Halozyme Therapeutics' ENHANZE® drug-delivery technology (see U.S. Pat. No. 7,767,429, which is incorporated by reference herein in its entirety). ENHANZE® uses a co-formulation of an antibody with recombinant human hyaluronidase enzyme (rHuPH20), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, anti-oxidant, aqueous and non-aqueous carriers, and/or adjuvants such as preservatives, wetting agents, emulsifying agents and dispersing agents. In some aspects, the pharmaceutical composition for the present disclosure can further comprise recombinant human hyaluronidase enzyme, e.g., rHuPH20.

Treatment is continued as long as clinical benefit is observed or until unacceptable toxicity or disease progression occurs. Dosage and frequency vary depending on the half-life of the inhibitor, antibody, and/or agent in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients (i.e., inhibitors, antibodies, and/or agents) in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

Provided herein is a pharmaceutical composition comprising an anti-LAG-3 antibody and an anti-PD-1 antibody as described herein at any of the doses or combinations of doses described herein.

In some aspects, the pharmaceutical composition is for treating a human subject with lung cancer as described herein (e.g., as a maintenance therapy).

In some aspects, a method for treating a human subject with lung cancer as described herein comprises administering a pharmaceutical composition as described herein.

In some aspects, the pharmaceutical composition comprises a dose of relatlimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is nivolumab.

In some aspects, the pharmaceutical composition comprises a dose of favezelimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is pembrolizumab.

In some aspects, the pharmaceutical composition comprises a dose of fianlimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is cemiplimab.

In some aspects, the pharmaceutical composition comprises a dose of ieramilimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is spartalizumab.

In some aspects, the pharmaceutical composition comprises a ratio of anti-LAG-3 antibody to anti-PD-1 antibody of about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1.

In some aspects, the pharmaceutical composition comprises a ratio of anti-LAG-3 antibody to anti-PD-1 antibody of about 1:3.

In some aspects, the pharmaceutical composition comprises a ratio of anti-LAG-3 antibody to anti-PD-1 antibody of about 1:1

In some aspects, the pharmaceutical composition comprises a ratio of anti-LAG-3 antibody to anti-PD-1 antibody of about 2:1.

In some aspects, the pharmaceutical composition comprises a ratio of anti-LAG-3 antibody to anti-PD-1 antibody of about 4:1.

In some aspects, the total amount of anti-LAG-3 and anti-PD-1 antibodies in the pharmaceutical composition is about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 205 mg/mL, about 210 mg/mL, about 215 mg/mL, about 220 mg/mL, about 225 mg/mL, about 230 mg/mL, about 235 mg/mL, about 240 mg/mL, about 245 mg/mL, about 250 mg/mL, about 255 mg/mL, about 260 mg/mL, about 265 mg/mL, about 270 mg/mL, about 275 mg/mL, about 280 mg/mL, about 285 mg/mL, about 290 mg/mL, about 295 mg/mL, about 300 mg/mL, about 305 mg/mL, about 310 mg/mL, about 315 mg/mL, about 320 mg/mL, about 325 mg/mL, about 330 mg/mL, about 335 mg/mL, about 340 mg/mL, about 345 mg/mL, about 350 mg/mL, about 355 mg/mL, about 360 mg/mL, about 365 mg/mL, about 370 mg/mL, about 375 mg/mL, about 380 mg/mL, about 385 mg/mL, about 390 mg/mL, about 395 mg/mL, about 400 mg/mL, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1580 mg, about 1590 mg, about 1600 mg, about 1610 mg, about 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, or about 1780 mg.

In some aspects, the total amount of anti-LAG-3 and anti-PD-1 antibodies in the pharmaceutical composition is about 25 mg/mL.

In some aspects, the total amount of anti-LAG-3 and anti-PD-1 antibodies in the pharmaceutical composition is about 50 mg/mL.

In some aspects, the total amount of anti-LAG-3 and anti-PD-1 antibodies in the pharmaceutical composition is about 150 mg/mL.

In some aspects, the total amount of anti-LAG-3 and anti-PD-1 antibodies in the pharmaceutical composition is about 50 mg.

In some aspects, the total amount of anti-LAG-3 and anti-PD-1 antibodies in the pharmaceutical composition is about 320 mg.

In some aspects, the total amount of anti-LAG-3 and anti-PD-1 antibodies in the pharmaceutical composition is about 640 mg.

In some aspects, the total amount of anti-LAG-3 and anti-PD-1 antibodies in the pharmaceutical composition is about 720 mg.

In some aspects, the total amount of anti-LAG-3 and anti-PD-1 antibodies in the pharmaceutical composition is about 960 mg.

In some aspects, the total amount of anti-LAG-3 and anti-PD-1 antibodies in the pharmaceutical composition is about 1000 mg.

In some aspects, the total amount of anti-LAG-3 and anti-PD-1 antibodies in the pharmaceutical composition is about 1080 mg.

In some aspects, the total amount of anti-LAG-3 and anti-PD-1 antibodies in the pharmaceutical composition is about 1440 mg.

In some aspects, the pharmaceutical composition comprises about 10 mg/mL, about 12.5 mg/mL, about 15 mg/mL, about 17.5 mg/mL, about 20 mg/mL, about 22.5 mg/mL, about 25 mg/mL, about 27.5 mg/mL, about 30 mg/mL, about 32.5 mg/mL, about 35 mg/mL, about 37.5 mg/mL, about 40 mg/mL, about 42.5 mg/mL, about 45 mg/mL, about 47.5 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 7 mg, about 21 mg, about 40 mg, about 70 mg, about 80 mg, about 160 mg, about 200 mg, about 210 mg, about 300 mg, about 400 mg, about 480 mg, about 500 mg, about 600 mg, about 700 mg, about 800 mg, about 900 mg, about 960 mg, about 1000 mg, about 1100 mg, about 1200 mg, or about 1300 mg of an anti-LAG-3 antibody. In some aspects, the pharmaceutical composition comprises about 5 mg/mL, about 10 mg/mL, about 12.5 mg/mL, about 15 mg/mL, about 17.5 mg/mL, about 20 mg/mL, about 22.5 mg/mL, about 25 mg/mL, about 27.5 mg/ml, about 30 mg/mL, about 32.5 mg/mL, about 35 mg/mL, about 37.5 mg/mL, about 40 mg/mL, about 42.5 mg/mL, about 45 mg/mL, about 47.5 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 10 mg, about 40 mg, about 100 mg, about 200 mg, about 240 mg, about 300 mg, about 350 mg, about 360 mg, about 400 mg, or about 480 mg of an anti-PD-1 antibody.

In some aspects, the pharmaceutical composition comprises about 12.5 mg/mL of an anti-LAG-3 antibody and about 37.5 mg/mL of an anti-PD-1 antibody.

In some aspects, the pharmaceutical composition comprises about 20 mg/mL of an anti-LAG-3 antibody and about 5 mg/mL of an anti-PD-1 antibody.

In some aspects, the pharmaceutical composition comprises about 75 mg/mL of an anti-LAG-3 antibody and about 75 mg/mL of an anti-PD-1 antibody.

In some aspects, the pharmaceutical composition comprises about 100 mg/ml of an anti-LAG-3 antibody and about 50 mg/mL of an anti-PD-1 antibody.

In some aspects, the pharmaceutical composition comprises about 80 mg of an anti-LAG-3 antibody and about 240 mg of an anti-PD-1 antibody.

In some aspects, the pharmaceutical composition comprises about 160 mg of an anti-LAG-3 antibody and about 480 mg of an anti-PD-1 antibody.

In some aspects, the pharmaceutical composition comprises about 360 mg of an anti-LAG-3 antibody and about 360 mg of an anti-PD-1 antibody.

In some aspects, the pharmaceutical composition comprises about 480 mg of an anti-LAG-3 antibody and about 480 mg of an anti-PD-1 antibody.

In some aspects, the pharmaceutical composition comprises about 720 mg of an anti-LAG-3 antibody and about 360 mg of an anti-PD-1 antibody.

In some aspects, the pharmaceutical composition comprises about 800 mg of an anti-LAG-3 antibody and about 200 mg of an anti-PD-1 antibody.

In some aspects, the pharmaceutical composition comprises about 960 mg of an anti-LAG-3 antibody and about 480 mg of an anti-PD-1 antibody.

In some aspects, the pharmaceutical composition comprises from about 5 mM to about 50 mM of histidine, from about 50 mM to about 300 mM of sucrose, from about 5 μM to about 1 mM of diethylenetriaminepentaacetic acid (DTPA) or ethylenediaminetetraacetic acid (EDTA), and from about 0.001% to about 1% (w/v) of polysorbate or poloxamer (e.g., polysorbate 80 (PS80), polysorbate 20 (PS20), poloxamer 188 (PX188), or any combination thereof).

In some aspects, the pharmaceutical composition comprises about 20 mM histidine, about 250 mM sucrose, about 50 μM DTPA, and 0.05% PS80.

In some aspects, the pH of the pharmaceutical composition is from about 5 to about 6.5. In some aspects, the pH is about 5.3 to about 6.3. In some aspects, the pH is 5.8. In some aspects, the pHis 5.7.

Provided herein is a vial, syringe, or intravenous bag comprising a pharmaceutical composition as described herein. In some aspects, the disclosure includes an autoinjector comprising a pharmaceutical composition described herein.

In some aspects, a vial comprises a pharmaceutical composition as described herein, and the vial further comprises a stopper and a seal. In some aspects, the total volume in the vial is about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL.

Also within the scope of the present invention are kits for treating a human subject with lung cancer comprising any of the antibodies, therapeutic agents, and/or anti-cancer therapies described herein.

Kits typically include a label indicating the intended use of the contents of the kit and instructions for use. The term “label” includes any writing, or recorded material supplied on or with the kit, or which otherwise accompanies the kit. In some aspects, the kit comprises instructions for using the kit components in a method for treating a human subject afflicted with lung cancer.

The antibodies can be provided at any of the doses or combinations of doses described herein.

In some aspects, the kit comprises a dose of relatlimab and a dose of an anti-PD-1 antibody as described herein or two different doses of an anti-PD-1 antibody as described herein, wherein the two different doses can be of the same or different anti-PD-1 antibodies.

In some aspects, a dose of an anti-LAG-3 antibody and an anti-PD-1 antibody as disclosed herein are co-packaged in a single unit dosage form.

In some aspects, all doses of antibodies are packaged as separate unit dosage forms.

In some aspects, the kit further comprises therapeutic agents for one or more PDCTs as disclosed herein. In some aspects, the therapeutic agents for one or more PDCTs are carboplatin and paclitaxel, carboplatin and albumin-bound paclitaxel, carboplatin and pemetrexed, and/or cisplatin and pemetrexed. In some aspects, the therapeutic agents are carboplatin, cisplatin, paclitaxel, albumin-bound paclitaxel, and pemetrexed.

All of the references cited above, as well as all references cited herein, are incorporated herein by reference in their entireties.

The following examples are offered by way of illustration and not by way of limitation.

### EXAMPLES

### Example 1

Anti-PD-1 Antibody Plus Concurrent Chemoradiotherapy Followed by Anti-LAG-3 Antibody in Combination with Anti-PD-1 Antibody for Treatment of Lung Cancer

A multi-center, double-blind, randomized Phase 3 global trial will evaluate the efficacy and safety of concurrent chemoradiotherapy (CCRT) plus nivolumab followed by maintenance therapy with a nivolumab and relatlimab fixed dose combination (FDC) versus CCRT plus placebo followed by maintenance therapy with durvalumab in subjects with untreated, unresectable Stage IIIA, IIIB, IIIC, locally advanced non-small cell lung cancer (NSCLC).

Approximately 850 male and female adults ≥18 years of age, or the local age of consent in the jurisdiction in which the study is taking place at the time of signing the informed consent form, will be randomized (1:1) across 2 treatment arms (Arms A and B) and stratified by PD-L1 expression level (≥1%, <1%, or non-quantifiable [NQ]) as well as disease stage (IIIA, IIIB, IIIC) per American Joint Committee on Cancer (AJCC) 8th edition of TNM (tumor, node, metastases) in lung cancer (Amin et al., AJCC Cancer Staging Manual (8th edition). Springer International Publishing: American Joint Commission on Cancer; 2017).

Each Arm will include a CCRT phase, a recovery period, and a maintenance phase. Arm A
CCRT phase (three 21-day cycles): nivolumab (360 mg IV infusion over 30±5 minutes every three weeks (Q3W)±3 days and platinum doublet chemotherapy (PDCT, IV Q3W) in each of Cycles 1, 2, and 3 plus radiotherapy (dose of 60-66 Gy) for Cycles 2 and 3.
Maintenance phase (28-day cycles for up to one year of treatment): nivolumab 480 mg+relatlimab 480 mg FDC IV infusion over 60 minutes Q4W

### Arm B

CCRT phase (three 21-day cycles): placebo (IV infusion over 30±5 minutes Q3W±3 days) and PDCT (IV Q3W) in each of Cycles 1, 2, and 3 plus radiotherapy (dose of 60-66 Gy) for Cycles 2 and 3.
Maintenance phase (28-day cycles for up to one year of treatment): durvalumab 1500 mg IV infusion over 60 minutes Q4W.

During the CCRT phase, nivolumab (Arm A) or placebo (Arm B) will be administered on Day 1 of each cycle prior to administration of chemotherapy (i.e., PDCT).

Subjects with squamous histology NSCLC will receive cisplatin/etoposide or carboplatin/paclitaxel as the PDCT during the CCRT phase.

Subjects with non-squamous histology NSCLC will receive either cisplatin/etoposide, carboplatin/paclitaxel, or cisplatin/pemetrexed as the PDCT during the CCRT phase.

For cisplatin/etoposide PDCT, 80 mg/m² cisplatin IV infusion over 60 minutes (or per local standard) will be administered on Day 1 of each of Cycles 1, 2, and 3, and 100 mg/m² etoposide IV infusion over 60 minutes (or per local standard) will be administered on Days 1, 2, and 3 of each of Cycles 1, 2, and 3. Days 2 and 3 may be interrupted, delayed, or discontinued depending on how well the participant tolerates the treatment and per investigator's discretion. Etoposide will be infused before cisplatin. If cisplatin cannot be tolerated, cisplatin may be replaced with carboplatin at a target area under the concentration-time curve (AUC) of 5 mg/mL·min IV infusion over 30 minutes (or per local standard)

For carboplatin/paclitaxel PDCT, carboplatin AUC 5 mg/mL·min or 6 mg/mL·min IV infusion over 30 minutes (or per local standard) and 175 or 200 mg/m² paclitaxel IV infusion over 180 minutes (or per local standard) will be administered on Day 1 of Cycle 1, while carboplatin AUC 2 mg/mL·min IV infusion over 30 minutes (or per local standard) and 45 or 50 mg/m² paclitaxel IV infusion over 60 minutes (or per local standard) will be administered on Days 1, 8, and 15 of Cycles 2 and 3. Paclitaxel infusions will be administered before carboplatin for Cycles 1, 2 and 3. Days 8 and 15 of Cycles 2 and 3 may be interrupted, delayed, or discontinued depending on how well the participant tolerates the treatment and per investigator's discretion. The carboplatin dose must be calculated according to Calvert formula. When calculating creatinine clearance (CrCl), the formula of Cockcroft-Gault (see below) should be used or per local standard:
Calvert Formula: Dose (in mg)=Target AUC×(glomerular filtration rate+25). Cockcroft-Gault: CrCl (mL/min)=([140-age (years)×actual body weight (kg)]/[72× serum creatinine (mg/dL)])×{0.85 if female}

For cisplatin/pemetrexed PDCT, 75 mg/m² cisplatin IV infusion over 60 minutes (or per local standard) and 500 mg/m² pemetrexed IV infusion over 10 minutes (or per local standard) will be administered on Day 1 of each of Cycles 1, 2, and 3. Pemetrexed will be infused before cisplatin. If cisplatin cannot be tolerated, cisplatin may be replaced with carboplatin at AUC 5 mg/mL·min IV infusion over 30 minutes (or per local standard). If pemetrexed cannot be tolerated, pemetrexed may be replaced with etoposide.

The recovery period in each Arm is the time between the CCRT phase and the maintenance phase. The recovery period is variable depending on adverse events/serious adverse events and treatment required, but is expected to last approximately 3 to 6 weeks, but no less than 18 days from the previous dosing of nivolumab (Arm A) or placebo (Arm B) during the Q3W cycles.

The radiotherapy in each Arm will be administered at a dose of 60-66 Gy in 30-33 daily fractions of 2 Gy, typically on a 5 days on/2 days off schedule as appropriate, over 6 to 7 weeks. Each subject will receive thoracic radiotherapy in form of intensity-modulated radiotherapy (IMRT), volumetric-modulated arc therapy (VMAT), or 3-dimensional conformal radiation therapy (3DRT). Irradiation commences on Day 1 of Cycle 2 of the CCRT phase.

### Key Inclusion Criteria for CCRT

Locally advanced Stage IIIA, IIIB, or IIIC (T1-2 N2-3 M0, T3 N1-3 M0, or T4 N0-3 M0) pathologically confirmed (including cytology) NSCLC per AJCC 8th edition of TNM in lung cancer eligible for definitive CCRT. Participants who are not planned for potential curative surgical resection are eligible.
Eastern Cooperative Oncology Group performance status≤1.
Treatment-naïve, with no prior local or systemic anticancer therapy given as primary therapy for locally advanced disease.
Measurable disease per Response Evaluation Criteria in Solid Tumors version 1.1 (RECIST v1.1) criteria.
All participants must have sufficient fresh or archival tumor tissue available for biomarker analyses.
Investigators shall counsel participants who are women of childbearing potential (WOCBP), and male participants who are sexually active with WOCBP, on the importance of pregnancy prevention, the implications of an unexpected pregnancy, and the potential of fetal toxicity occurring due to transmission of study intervention, present in seminal fluid, to a developing fetus, even if the participant has undergone a successful vasectomy or if the partner is pregnant.

### Key Exclusion Criteria for CCRT

Participants with a history of myocarditis, regardless of etiology.
Concurrent malignancy (present during screening) requiring treatment or history of prior malignancy active within 2 years prior to randomization.
Participants with an active, known, or suspected autoimmune disease.
Participants with a condition requiring systemic treatment with either corticosteroids (>10 mg daily prednisone equivalent) or other immunosuppressive medications within 14 days of start of randomization.
Presence of pleural/pericardial effusion on computed tomography (CT) scan and/or x-ray.

### Key Inclusion Criteria for Maintenance

No BICR-confirmed progressive disease per RECIST v1.1 during CCRT or during Recovery Period.
No current or prior use of immunosuppressive medication (not to exceed 10 mg/day of daily prednisone or equivalent) within 14 days prior to the first dose of maintenance.
Toxicity from CCRT must resolve to Grade 1 or baseline (except for Grade 2 fatigue, esophagitis, or alopecia).

### Key Exclusion Criteria for Maintenance

Serum creatinine >1.5× upper limit of normal (ULN), unless creatinine clearance ≥40 mL/min (measured or calculated using the Cockcroft-Gault formula).
Aspartate aminotransferase/alanine aminotransferase >3.0×ULN.
Total bilirubin >1.5×ULN (except participants with Gilbert syndrome who must have a total bilirubin level of <3.0×ULN).
Troponin T or I >2× institutional ULN.

The study will demonstrate that nivolumab plus CCRT followed by maintenance with nivolumab and relatlimab FDC (Arm A) compared to CCRT plus placebo followed by maintenance with durvalumab (Arm B) improves progression-free survival (PFS) in participants with locally advanced, previously untreated, unresectable, Stage III NSCLC.

SEQUENCES
Heavy Chain Amino Acid Sequence; Anti-LAG-3 mAb (BMS-986016)
SEQ ID NO: 1
QVQLQQWGAGLLKPSETLSLTCAVYGGSFSDYYWNWIRQPPGKGLEWIGEINHRGSTNSNPSLKS
RVTLSLDTSKNQFSLKLRSVTAADTAVYYCAFGYSDYEYNWFDPWGQGTLVTVSSASTKGPSVFP
LAPCSRSTSESTAALGCLVKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
Light Chain Amino Acid Sequence; Anti-LAG-3 mAb (BMS-986016)
SEQ ID NO: 2
EIVLTQSPATLSLSPGERATLSCRASQSISSYLAWYQQKPGQAPRLLIYDASNRATGIPARFSGS
GSGTDFTLTISSLEPEDFAVYYCQQRSNWPLTFGQGTNLEIKRTVAAPSVFIFPPSDEQLKSGTA
SVVCLLNNFYPREAKVQWKVDNALQSGNSQESVTEQDSKDSTYSLSSTLTLSKADYEKHKVYACE
VTHQGLSSPVTKSFNRGEC
Heavy Chain Variable Region (VH) Amino Acid Sequence; Anti-LAG-3 mAb
(BMS-986016)
SEQ ID NO: 3
QVQLQQWGAGLLKPSETLSLTCAVYGGSFSDYYWNWIRQPPGKGLEWIGEINHRGSTNSNPSLKS
RVTLSLDTSKNQFSLKLRSVTAADTAVYYCAFGYSDYEYNWFDPWGQGTLVTVSS
Light Chain Variable Region (VL) Amino Acid Sequence;
Anti-LAG-3 mAb (BMS-986016)
SEQ ID NO: 4
EIVLTQSPATLSLSPGERATLSCRASQSISSYLAWYQQKPGQAPRLLIYDASNRATGIPARFSGS
GSGTDFTLTISSLEPEDFAVYYCQQRSNWPLTFGQGTNLEIK
Heavy Chain CDR1 Amino Acid Sequence; Anti-LAG-3 mAb (BMS-986016)
SEQ ID NO: 5
DYYWN
Heavy Chain CDR2 Amino Acid Sequence; Anti-LAG-3 mAb (BMS-986016)
SEQ ID NO: 6
EINHRGSTNSNPSLKS
Heavy Chain CDR3 Amino Acid Sequence; Anti-LAG-3 mAb (BMS-986016)
SEQ ID NO: 7
GYSDYEYNWFDP
Light Chain CDR1 Amino Acid Sequence; Anti-LAG-3 mAb (BMS-986016)
SEQ ID NO: 8
RASQSISSYLA
Light Chain CDR2 Amino Acid Sequence; Anti-LAG-3 mAb (BMS-986016)
SEQ ID NO: 9
DASNRAT
Light Chain CDR3 Amino Acid Sequence; Anti-LAG-3 mAb (BMS-986016)
SEQ ID NO: 10
QQRSNWPLT
Heavy Chain Amino Acid Sequence; Anti-PD-1 mAb (BMS-936558)
SEQ ID NO: 11
QVQLVESGGGVVQPGRSLRLDCKASGITFSNSGMHWVRQAPGKGLEWVAVIWYDGSKRYYADSVK
GRFTISRDNSKNTLFLQMNSLRAEDTAVYYCATNDDYWGQGTLVTVSSASTKGPSVFPLAPCSRS
TSESTAALGCLVKDYFPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGK
Light Chain Amino Acid Sequence; Anti-PD-1 mAb (BMS-936558)
SEQ ID NO: 12
EIVLTQSPATLSLSPGERATLSCRASQSVSSYLAWYQQKPGQAPRLLIYDASNRATGIPARFSGS
GSGTDFTLTISSLEPEDFAVYYCQQSSNWPRTFGQGTKVEIKRTVAAPSVFIFPPSDEQLKSGTA
SVVCLLNNFYPREAKVQWKVDNALQSGNSQESVTEQDSKDSTYSLSSTLLSKADYEKHKVYACEV
THQGLSSPVTKSFNRGEC
Heavy Chain Variable Region (VH) Amino Acid Sequence; Anti-PD-1 mAb
(BMS-936558)
SEQ ID NO: 13
QVQLVESGGGVVQPGRSLRLDCKASGITFSNSGMHWVRQAPGKGLEWVAVIWYDGSKRYYADSVK
GRFTISRDNSKNTLFLQMNSLRAEDTAVYYCATNDDYWGQGTLVTVSS
Light Chain Variable Region (VL) Amino Acid Sequence; Anti-PD-1 mAb
(BMS-936558)
SEQ ID NO: 14
EIVLTQSPATLSLSPGERATLSCRASQSVSSYLAWYQQKPGQAPRLLIYDASNRATGIPARFSGS
GSGTDFTLTISSLEPEDFAVYYCQQSSNWPRTFGQGTKVEIK
Heavy Chain CDR1 Amino Acid Sequence; Anti-PD-1 mAb (BMS-936558)
SEQ ID NO: 15
NSGMH
Heavy Chain CDR2 Amino Acid Sequence; Anti-PD-1 mAb (BMS-936558)
SEQ ID NO: 16
VIWYDGSKRYYADSVKG
Heavy Chain CDR3 Amino Acid Sequence; Anti-PD-1 mAb (BMS-936558)
SEQ ID NO: 17
NDDY
Light Chain CDR1 Amino Acid Sequence; Anti-PD-1 mAb (BMS-936558)
SEQ ID NO: 18
RASQSVSSYLA
Light Chain CDR2 Amino Acid Sequence; Anti-PD-1 mAb (BMS-936558)
SEQ ID NO: 19
DASNRAT
Light Chain CDR3 Amino Acid Sequence; Anti-PD-1 mAb (BMS-936558)
SEQ ID NO: 20
QQSSNWPRT
Heavy Chain Amino Acid Sequence; Anti-LAG-3 mAb (BMS-986016) without
terminal lysine
SEQ ID NO: 21
QVQLQQWGAGLLKPSETLSLTCAVYGGSFSDYYWNWIRQPPGKGLEWIGEINHRGSTNSNPSLKS
RVTLSLDTSKNQFSLKLRSVTAADTAVYYCAFGYSDYEYNWFDPWGQGTLVTVSSASTKGPSVFP
LAPCSRSTSESTAALGCLVKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
Lymphocyte Activation Gene 3 Protein Amino Acid Sequence
(Homo Sapiens, NP_002277)
SEQ ID NO: 22
MWEAQFLGLLFLQPLWVAPVKPLQPGAEVPVVWAQEGAPAQLPCSPTIPLQDLSLLRRAGVTWQH
QPDSGPPAAAPGHPLAPGPHPAAPSSWGPRPRRYTVLSVGPGGLRSGRLPLQPRVQLDERGRQRG
DFSLWLRPARRADAGEYRAAVHLRDRALSCRLRLRLGQASMTASPPGSLRASDWVILNCSFSRPD
RPASVHWFRNRGQGRVPVRESPHHHLAESFLFLPQVSPMDSGPWGCILTYRDGFNVSIMYNLTVL
GLEPPTPLTVYAGAGSRVGLPCRLPAGVGTRSFLTAKWTPPGGGPDLLVTGDNGDFTLRLEDVSQ
AQAGTYTCHIHLQEQQLNATVTLAIITVTPKSFGSPGSLGKLLCEVTPVSGQERFVWSSLDTPSQ
RSFSGPWLEAQEAQLLSQPWQCQLYQGERLLGAAVYFTELSSPGAQRSGRAPGALPAGHLLLFLI
LGVLSLLLLVTGAFGFHLWRRQWRPRRFSALEQGIHPPQAQSKIEELEQEPEPEPEPEPEPEPEP
EPEQL
Heavy Chain Amino Acid Sequence; Anti-LAG-3 mAb (REGN3767)
SEQ ID NO: 23
QVQLVESGGGVVQPGRSLRLSCVASGFTFSSYGMHWVRQAPGKGLEWVAI IWYDGSNKYY
ADSVKGRFTISRDNSKNTQYLQMNSLRAEDTAVYYCASVATSGDFDYYGMDVWGQGTTVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPPVAGP
SVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWQ
EGNVFSCSVMHEALHNHYTQKSLSLSLGK
Light Chain Amino Acid Sequence; Anti-LAG-3 mAb (REGN3767)
SEQ ID NO: 24
EIVLTQSPATLSLSPGERTTLSCRASQRISTYLAWYQQKPGQAPRLLIYDASKRATGIPA
RFSGSGSGTGFTLTISSLEPEDFAVYYCQQRSNWPLTFGGGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Heavy Chain Variable Region (VH) Amino Acid Sequence; Anti-LAG-3 mAb
(REGN3767)
SEQ ID NO: 25
QVQLVESGGGVVQPGRSLRLSCVASGFTFSSYGMHWVRQAPGKGLEWVAIIWYDGSNKYY
ADSVKGRFTISRDNSKNTQYLQMNSLRAEDTAVYYCASVATSGDFDYYGMDVWGQGTTVT
VSS
Light Chain Variable Region (VL) Amino Acid Sequence; Anti-LAG-3 mAb
(REGN3767)
SEQ ID NO: 26
EIVLTQSPATLSLSPGERTTLSCRASQRISTYLAWYQQKPGQAPRLLIYDASKRATGIPA
RFSGSGSGTGFTLTISSLEPEDFAVYYCQQRSNWPLTFGGGTKVEIK
Heavy Chain CDR1 Amino Acid Sequence; Anti-LAG-3 mAb (REGN3767)
SEQ ID NO: 27
GFTFSSYG
Heavy Chain CDR2 Amino Acid Sequence; Anti-LAG-3 mAb (REGN3767)
SEQ ID NO: 28
IWYDGSNK
Heavy Chain CDR3 Amino Acid Sequence; Anti-LAG-3 mAb (REGN3767)
SEQ ID NO: 29
ASVATSGDFDYYGMDV
Light Chain CDR1 Amino Acid Sequence; Anti-LAG-3 mAb (REGN3767)
SEQ ID NO: 30
QRISTY
Light Chain CDR2 Amino Acid Sequence; Anti-LAG-3 mAb (REGN3767)
SEQ ID NO: 31
DAS
Light Chain CDR3 Amino Acid Sequence; Anti-LAG-3 mAb (REGN3767)
SEQ ID NO: 32
QQRSNWPLT
Heavy Chain Amino Acid Sequence; Anti-PD-1 mAb (REGN2810)
SEQ ID NO: 33
EVQLLESGGVLVQPGGSLRLSCAASGFTFSNFGMTWVRQAPGKGLEWVSGISGGGRDTYF
ADSVKGRFTISRDNSKNTLYLQMNSLKGEDTAVYYCVKWGNIYFDYWGQGTLVTVSSAST
KGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFLGGPSVFLF
PPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTVDKSRWQEGNVF
SCSVMHEALHNHYTQKSLSLSLGK
Light Chain Amino Acid Sequence; Anti-PD-1 mAb (REGN2810)
SEQ ID NO: 34
DIQMTQSPSSLSASVGDSITITCRASLSINTFLNWYQQKPGKAPNLLIYAASSLHGGVPS
RFSGSGSGTDFTLTIRTLQPEDFATYYCQQSSNTPFTFGPGTVVDFRRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Heavy Chain Variable Region (VH) Amino Acid Sequence;
Anti-PD-1 mAb (REGN2810)
SEQ ID NO: 35
EVQLLESGGVLVQPGGSLRLSCAASGFTFSNFGMTWVRQAPGKGLEWVSGISGGGRDTYF
ADSVKGRFTISRDNSKNTLYLQMNSLKGEDTAVYYCVKWGNIYFDYWGQGTLVTVSS
Light Chain Variable Region (VL) Amino Acid Sequence; Anti-PD-1 mAb
(REGN2810)
SEQ ID NO: 36
DIQMTQSPSSLSASVGDSITITCRASLSINTFLNWYQQKPGKAPNLLIYAASSLHGGVPS
RFSGSGSGTDFTLTIRTLQPEDFATYYCQQSSNTPFTFGPGTVVDFR
Heavy Chain CDR1 Amino Acid Sequence; Anti-PD-1 mAb (REGN2810)
SEQ ID NO: 37
GFTFSNFG
Heavy Chain CDR2 Amino Acid Sequence; Anti-PD-1 mAb (REGN2810)
SEQ ID NO: 38
ISGGGRDT
Heavy Chain CDR3 Amino Acid Sequence; Anti-PD-1 mAb (REGN2810)
SEQ ID NO: 39
VKWGNIYFDY
Light Chain CDR1 Amino Acid Sequence; Anti-PD-1 mAb (REGN2810)
SEQ ID NO: 40
LSINTF
Light Chain CDR2 Amino Acid Sequence; Anti-PD-1 mAb (REGN2810)
SEQ ID NO: 41
AAS
Light Chain CDR3 Amino Acid Sequence; Anti-PD-1 mAb (REGN2810)
SEQ ID NO: 42
QQSSNTPFT
Heavy Chain Amino Acid Sequence; Anti-LAG-3 mAb (LAG525)
SEQ ID NO: 43
QVQLVQSGAEVKKPGASVKVSCKASGFTLTNYGMNWVRQARGQRLEWIGWINTDTGEPTY
ADDFKGRFVFSLDTSVSTAYLQISSLKAEDTAVYYCARNPPYYYGTNNAEAMDYWGQGTT
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDKS
RWQEGNVFSCSVMHEALHNHYTQKSLSLSLG
Heavy Chain Amino Acid Sequence; Anti-LAG-3 mAb (LAG525)
SEQ ID NO: 44
QVQLVQSGAEVKKPGASVKVSCKASGFTLTNYGMNWVRQAPGQGLEWMGWINTDTGEPTY
ADDFKGRFVFSLDTSVSTAYLQISSLKAEDTAVYYCARNPPYYYGTNNAEAMDYWGQGTT
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDKS
RWQEGNVFSCSVMHEALHNHYTQKSLSLSLG
Light Chain Amino Acid Sequence; Anti-LAG-3 mAb (LAG525)
SEQ ID NO: 45
DIQMTQSPSSLSASVGDRVTITCSSSQDISNYLNWYLQKPGQSPQLLIYYTSTLHLGVPS
RFSGSGSGTEFTLTISSLQPDDFATYYCQQYYNLPWTFGQGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Light Chain Amino Acid Sequence; Anti-LAG-3 mAb (LAG525)
SEQ ID NO: 46
DIQMTQSPSSLSASVGDRVTITCSSSQDISNYLNWYQQKPGKAPKLLIYYTSTLHLGIPP
RFSGSGYGTDFTLTINNIESEDAAYYFCQQYYNLPWTFGQGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Heavy Chain Variable Region (VH) Amino Acid Sequence;
Anti-LAG-3 mAb (LAG525)
SEQ ID NO: 47
QVQLVQSGAEVKKPGASVKVSCKASGFTLTNYGMNWVRQARGQRLEWIGWINTDTGEPTY
ADDFKGRFVFSLDTSVSTAYLQISSLKAEDTAVYYCARNPPYYYGTNNAEAMDYWGQGTT
VTVSS
Heavy Chain Variable Region (VH) Amino Acid Sequence;
Anti-LAG-3 mAb (LAG525)
SEQ ID NO: 48
QVQLVQSGAEVKKPGASVKVSCKASGFTLTNYGMNWVRQAPGQGLEWMGWINTDTGEPTY
ADDFKGRFVFSLDTSVSTAYLQISSLKAEDTAVYYCARNPPYYYGTNNAEAMDYWGQGTT
VTVSS
Light Chain Variable Region (VL) Amino Acid Sequence;
Anti-LAG-3 mAb (LAG525)
SEQ ID NO: 49
DIQMTQSPSSLSASVGDRVTITCSSSQDISNYLNWYLQKPGQSPQLLIYYTSTLHLGVPS
RFSGSGSGTEFTLTISSLQPDDFATYYCQQYYNLPWTFGQGTKVEIK
Light Chain Variable Region (VL) Amino Acid Sequence;
Anti-LAG-3 mAb (LAG525)
SEQ ID NO: 50
DIQMTQSPSSLSASVGDRVTITCSSSQDISNYLNWYQQKPGKAPKLLIYYTSTLHLGIPP
RFSGSGYGTDFTLTINNIESEDAAYYFCQQYYNLPWTFGQGTKVEIK
Heavy Chain CDR1 Amino Acid Sequence; Anti-LAG-3 mAb (LAG525)
SEQ ID NO: 51
NYGMN
Heavy Chain CDR2 Amino Acid Sequence; Anti-LAG-3 mAb (LAG525)
SEQ ID NO: 52
WINTDTGEPTYADDFKG
Heavy Chain CDR3 Amino Acid Sequence; Anti-LAG-3 mAb (LAG525)
SEQ ID NO: 53
NPPYYYGTNNAEAMDY
Light Chain CDR1 Amino Acid Sequence; Anti-LAG-3 mAb (LAG525)
SEQ ID NO: 54
SSSQDISNYLN
Light Chain CDR2 Amino Acid Sequence; Anti-LAG-3 mAb (LAG525)
SEQ ID NO: 55
YTSTLHL
Light Chain CDR3 Amino Acid Sequence; Anti-LAG-3 mAb (LAG525)
SEQ ID NO: 56
QQYYNLPWT
Heavy Chain Amino Acid Sequence; Anti-PD-1 mAb (PDR001)
SEQ ID NO: 57
EVQLVQSGAEVKKPGESLRISCKGSGYTFTTYWMHWVRQATGQGLEWMGNIYPGTGGSNF
DEKFKNRVTITADKSTSTAYMELSSLRSEDTAVYYCTRWTTGTGAYWGQGTTVTVSSAST
KGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFLGGPSVFLF
PPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTVDKSRWQEGNVF
SCSVMHEALHNHYTQKSLSLSLG
Light Chain Amino Acid Sequence; Anti-PD-1 mAb (PDR001)
SEQ ID NO: 58
EIVLTQSPATLSLSPGERATLSCKSSQSLLDSGNQKNFLTWYQQKPGQAPRLLIYWASTR
ESGVPSRFSGSGSGTDFTFTISSLEAEDAATYYCQNDYSYPYTFGQGTKVEIKRTVAAPS
VFIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Heavy Chain Variable Region (VH) Amino Acid Sequence;
Anti-PD-1 mAb (PDR001)
SEQ ID NO: 59
EVQLVQSGAEVKKPGESLRISCKGSGYTFTTYWMHWVRQATGQGLEWMGNIYPGTGGSNF
DEKFKNRVTITADKSTSTAYMELSSLRSEDTAVYYCTRWTTGTGAYWGQGTTVTVSS
Light Chain Variable Region (VL) Amino Acid Sequence;
Anti-PD-1 mAb (PDR001)
SEQ ID NO: 60
EIVLTQSPATLSLSPGERATLSCKSSQSLLDSGNQKNFLTWYQQKPGQAPRLLIYWASTR
ESGVPSRFSGSGSGTDFTFTISSLEAEDAATYYCQNDYSYPYTFGQGTKVEIK
Heavy Chain CDR1 Amino Acid Sequence; Anti-PD-1 mAb (PDR001)
SEQ ID NO: 61
TYWMH
Heavy Chain CDR2 Amino Acid Sequence; Anti-PD-1 mAb (PDR001)
SEQ ID NO: 62
NIYPGTGGSNFDEKFKN
Heavy Chain CDR3 Amino Acid Sequence; Anti-PD-1 mAb (PDR001)
SEQ ID NO: 63
WTTGTGAY
Light Chain CDR1 Amino Acid Sequence; Anti-PD-1 mAb (PDR001)
SEQ ID NO: 64
KSSQSLLDSGNQKNFLT
Light Chain CDR2 Amino Acid Sequence; Anti-PD-1 mAb (PDR001)
SEQ ID NO: 65
WASTRES
Light Chain CDR3 Amino Acid Sequence; Anti-PD-1 mAb (PDR001)
SEQ ID NO: 66
QNDYSYPYT
Heavy Chain Amino Acid Sequence; Anti-LAG-3 mAb (MK4280)
SEQ ID NO: 67
QMQLVQSGPEVKKPGTSVKVSCKASGYTFTDYNVDWVRQARGQRLEWIGDINPNDGGTIY
AQKFQERVTITVDKSTSTAYMELSSLRSEDTAVYYCARNYRWFGAMDHWGQGTTVTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGN
VFSCSVMHEALHNHYTQKSLSLSLGK
Light Chain Amino Acid Sequence; Anti-LAG-3 mAb (MK4280)
SEQ ID NO: 68
DIVMTQTPLSLSVTPGQPASISCKASQSLDYEGDSDMNWYLQKPGQPPQLLIYGASNLES
GVPDRFSGSGSGTDFTLKISRVEAEDVGVYYCQQSTEDPRTFGGGTKVEIKRTVAAPSVF
IFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Heavy Chain Variable Region (VH) Amino Acid Sequence;
Anti-LAG-3 mAb (MK4280)
SEQ ID NO: 69
QMQLVQSGPEVKKPGTSVKVSCKASGYTFTDYNVDWVRQARGQRLEWIGDINPNDGGTIY
AQKFQERVTITVDKSTSTAYMELSSLRSEDTAVYYCARNYRWFGAMDHWGQGTTVTVSS
Light Chain Variable Region (VL) Amino Acid Sequence;
Anti-LAG-3 Anti-LAG-3 mAb (MK4280)
SEQ ID NO: 70
DIVMTQTPLSLSVTPGQPASISCKASQSLDYEGDSDMNWYLQKPGQPPQLLIYGASNLES
GVPDRFSGSGSGTDFTLKISRVEAEDVGVYYCQQSTEDPRTFGGGTKVEIK
Heavy Chain CDR1 Amino Acid Sequence; Anti-LAG-3 mAb (MK4280)
SEQ ID NO: 71
DYNVD
Heavy Chain CDR2 Amino Acid Sequence; Anti-LAG-3 mAb (MK4280)
SEQ ID NO: 72
DINPNDGGTIYAQKFQE
Heavy Chain CDR3 Amino Acid Sequence; Anti-LAG-3 mAb (MK4280)
SEQ ID NO: 73
NYRWFGAMDH
Light Chain CDR1 Amino Acid Sequence; Anti-LAG-3 mAb (MK4280)
SEQ ID NO: 74
KASQSLDYEGDSDMN
Light Chain CDR2 Amino Acid Sequence; Anti-LAG-3 mAb (MK4280)
SEQ ID NO: 75
GASNLES
Light Chain CDR3 Amino Acid Sequence; Anti-LAG-3 mAb (MK4280)
SEQ ID NO: 76
QQSTEDPRT
Heavy Chain Amino Acid Sequence; Anti-PD-1 mAb (MK3475)
SEQ ID NO: 77
QVQLVQSGVEVKKPGASVKVSCKASGYTFTNYYMYWVRQAPGQGLEWMGGINPSNGGTNF
NEKFKNRVTLTTDSSTTTAYMELKSLQFDDTAVYYCARRDYRFDMGFDYWGQGTTVTVSS
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
Light Chain Amino Acid Sequence; Anti-PD-1 mAb (MK3475)
SEQ ID NO: 78
EIVLTQSPATLSLSPGERATLSCRASKGVSTSGYSYLHWYQQKPGQAPRLLIYLASYLES
GVPARFSGSGSGTDFTLTISSLEPEDFAVYYCQHSRDLPLTFGGGTKVEIKRTVAAPSVF
IFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Heavy Chain Variable Region (VH) Amino Acid Sequence;
Anti-PD-1 mAb (MK3475)
SEQ ID NO: 79
QVQLVQSGVEVKKPGASVKVSCKASGYTFTNYYMYWVRQAPGQGLEWMGGINPSNGGTNF
NEKFKNRVTLTTDSSTTTAYMELKSLQFDDTAVYYCARRDYRFDMGFDYWGQGTTVTVSS
Light Chain Variable Region (VL) Amino Acid Sequence;
Anti-PD-1 mAb (MK3475)
SEQ ID NO: 80
EIVLTQSPATLSLSPGERATLSCRASKGVSTSGYSYLHWYQQKPGQAPRLLIYLASYLES
GVPARFSGSGSGTDFTLTISSLEPEDFAVYYCQHSRDLPLTFGGGTKVEIK
Heavy Chain CDR1 Amino Acid Sequence; Anti-PD-1 mAb (MK3475)
SEQ ID NO: 81
NYYMY
Heavy Chain CDR2 Amino Acid Sequence; Anti-PD-1 mAb (MK3475)
SEQ ID NO: 82
GINPSNGGTNFNEKFKN
Heavy Chain CDR3 Amino Acid Sequence; Anti-PD-1 mAb (MK3475)
SEQ ID NO: 83
RDYRFDMGFDY
Light Chain CDR1 Amino Acid Sequence; Anti-PD-1 mAb (MK3475)
SEQ ID NO: 84
RASKGVSTSGYSYLH
Light Chain CDR2 Amino Acid Sequence; Anti-PD-1 mAb (MK3475)
SEQ ID NO: 85
LASYLES
Light Chain CDR3 Amino Acid Sequence; Anti-PD-1 mAb (MK3475)
SEQ ID NO: 86
QHSRDLPLT

What is claimed is:

1. A method of treating a human subject afflicted with lung cancer, the method comprising administering to the subject:
(a) a programmed death-1 (PD-1) pathway inhibitor and a concurrent chemoradiotherapy (CCRT), followed by
(b) a PD-1 pathway inhibitor and a lymphocyte activation gene-3 (LAG-3) antagonist.

2. The method of claim 1, further comprising providing a recovery period to the subject that begins upon completion of the administration in (a) and ends upon the start of the administration in (b).

3. The method of claim 2, wherein the recovery period is a duration sufficient for the subject to recover from toxicities associated with the CCRT other than fatigue, esophagitis, or alopecia.

4. The method of claim 2 or 3, wherein the recovery period is from about 1 week to about 12 weeks, about 1 week to about 9 weeks, about 1 week to about 6 weeks, about 2 weeks to about 12 weeks, about 2 weeks to about 9 weeks, about 2 weeks to about 6 weeks, about 3 weeks to about 12 weeks, about 3 weeks to about 9 weeks, about 3 weeks to about 6 weeks, about 18 days from final administration of the PD-1 pathway inhibitor in (a) to about 12 weeks, about 18 days from final administration of the PD-1 pathway inhibitor in (a) to about 9 weeks, or about 18 days from final administration of the PD-1 pathway inhibitor in (a) to about 6 weeks.

5. The method of any one of claims 1-4, wherein the method is a first line therapy.

6. The method of any one of claims 1-5, wherein the subject has not received a prior local or systemic anticancer therapy given as primary therapy for locally advanced disease.

7. The method of any one of claims 1-6, wherein the subject has not received a prior systemic therapy for cancer, the subject has not received a prior systemic therapy for lung cancer, or the subject has not received a prior systemic therapy for advanced or metastatic lung cancer.

8. The method of any one of claims 1-7, wherein the subject is naïve to prior immuno-oncology therapy, the subject is naïve to prior immuno-oncology therapy for lung cancer, or the lung cancer is naïve to prior immuno-oncology therapy.

9. The method of any one of claims 1-4, wherein the method is a second line therapy.

10. The method of any one of claims 1-4, wherein the method is a third line therapy.

11. The method of claim 9 or 10, wherein the subject has progressed on a prior therapy.

12. The method of any one of claims 9-11, wherein the lung cancer is recurrent following multi-modal therapy for locally advanced lung cancer.

13. The method of any one of claims 1-12, wherein the lung cancer is unresectable, advanced, recurrent, and/or metastatic.

14. The method of any one of claims 1-13, wherein the lung cancer comprises small cell lung cancer.

15. The method of any one of claims 1-14, wherein the lung cancer comprises non-small cell lung cancer (NSCLC).

16. The method of claim 15, wherein the NSCLC has a squamous or non-squamous histology.

17. The method of claim 15 or 16, wherein the NSCLC comprises locally advanced Stage IIIA, IIIB, or IIIC NSCLC.

18. The method of any one of claims 2-17, wherein the subject is free of progressive lung cancer during the CCRT or the recovery period.

19. The method of any one of claims 1-18, wherein the PD-1 pathway inhibitor in (a) and (b) are the same.

20. The method of any one of claims 1-18, wherein the PD-1 pathway inhibitor in (a) and (b) are different.

21. The method of any one of claims 1-20, wherein the PD-1 pathway inhibitor in (a) and/or (b) comprises an anti-PD-1 antibody and/or an anti-PD-L1 antibody.

22. The method of any one of claims 1-21, wherein the PD-1 pathway inhibitor in (a) and/or (b) comprises an anti-PD-1 antibody.

23. The method of claim 21 or 22, wherein the anti-PD-1 antibody comprises a full-length antibody.

24. The method of any one of claims 21-23, wherein the anti-PD-1 antibody comprises a monoclonal, human, humanized, chimeric, or multispecific antibody.

25. The method of claim 24, wherein the multispecific antibody comprises a dual-affinity re-targeting antibody (DART), a DVD-Ig, or bispecific antibody.

26. The method of claim 21 or 22, wherein the anti-PD-1 antibody comprises a F(ab′)₂ fragment, a Fab′ fragment, a Fab fragment, a Fv fragment, a scFv fragment, a dsFv fragment, a dAb fragment, or a single chain binding polypeptide.

27. The method of any one of claims 21-26, wherein the anti-PD-1 antibody comprises nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or an antigen binding portion thereof.

28. The method of any one of claims 21-27, wherein the anti-PD-1 antibody comprises nivolumab or an antigen binding portion thereof.

29. The method of any one of claims 21-28, wherein the anti-PD-1 antibody comprises CDR1, CDR2 and CDR3 domains of the heavy chain variable region having the sequence set forth in SEQ ID NO: 13, and CDR1, CDR2 and CDR3 domains of the light chain variable region having the sequence set forth in SEQ ID NO: 14.

30. The method of any one of claims 21-29, wherein the anti-PD-1 antibody comprises:
(a) a heavy chain variable region CDR1 comprising the sequence set forth in SEQ ID NO: 15;
(b) a heavy chain variable region CDR2 comprising the sequence set forth in SEQ ID NO: 16;
(c) a heavy chain variable region CDR3 comprising the sequence set forth in SEQ ID NO: 17;
(d) a light chain variable region CDR1 comprising the sequence set forth in SEQ ID NO: 18;
(e) a light chain variable region CDR2 comprising the sequence set forth in SEQ ID NO: 19; and
(f) a light chain variable region CDR3 comprising the sequence set forth in SEQ ID NO: 20.

31. The method of any one of claims 21-30 wherein the anti-PD-1 antibody comprises heavy and light chain variable regions comprising the sequences set forth in SEQ ID NOs: 13 and 14, respectively.

32. The method of any one of claim 21-25 or 27-31, wherein the anti-PD-1 antibody comprises heavy and light chains comprising the sequences as set forth in SEQ ID NOs: 11 and 12, respectively.

33. The method of any one of claims 1-20, wherein the PD-1 pathway inhibitor in (a) and/or (b) comprises a soluble PD-L2 polypeptide.

34. The method of claim 33, wherein the soluble PD-L2 polypeptide comprises a fusion polypeptide.

35. The method of claim 33 or 34, wherein the soluble PD-L2 polypeptide comprises a ligand binding fragment of the PD-L2 extracellular domain.

36. The method of any one of claims 33-35, wherein the soluble PD-L2 polypeptide further comprises a half-life extending moiety.

37. The method of claim 36, wherein the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof.

38. The method of any one of claims 33-37, wherein the soluble PD-L2 polypeptide comprises AMP-224.

39. The method of any one of claims 1-21, wherein the PD-1 pathway inhibitor in (a) and/or (b) comprises an anti-PD-L1 antibody.

40. The method of claim 21 or 39, wherein the anti-PD-L1 antibody comprises a full-length antibody.

41. The method of any one of claim 21 or 39-40, wherein the anti-PD-L1 antibody comprises a monoclonal, human, humanized, chimeric, or multispecific antibody.

42. The method of claim 41, wherein the multispecific antibody comprises a DART, a DVD-Ig, or bispecific antibody.

43. The method of any one of claim 21 or 39, wherein the anti-PD-L1 antibody comprises a F(ab′)₂ fragment, a Fab′ fragment, a Fab fragment, a Fv fragment, a scFv fragment, a dsFv fragment, a dAb fragment, or a single chain binding polypeptide.

44. The method of any one of claim 21 or 39-43, wherein the anti-PD-L1 antibody comprises BMS-936559, atezolizumab, durvalumab, avelumab, STI-1014, CX-072, KN035, LY3300054, BGB-A333, ICO 36, FAZ053, CK-301, or an antigen binding portion thereof.

45. The method of claim 1-20, wherein the PD-1 pathway inhibitor in (a) and/or (b) comprises BMS-986189.

46. The method of any one of claims 1-45, wherein the PD-1 pathway inhibitor in (a) and/or (b) is administered at a flat dose.

47. The method of any one of claims 1-46, wherein the PD-1 pathway inhibitor in (a) and/or (b) is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

48. The method of any one of claims 1-47, wherein the PD-1 pathway inhibitor in (a) and/or (b) is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

49. The method of any one of claims 1-45, wherein the PD-1 pathway inhibitor in (a) and/or (b) is administered as a weight-based dose.

50. The method of any one of claim 1-45 or 49, wherein the PD-1 pathway inhibitor in (a) and/or (b) is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

51. The method of any one of claim 1-45 or 49-50, wherein the PD-1 pathway inhibitor in (a) and/or (b) is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

52. The method of any one of claims 46-51, wherein the dose of the PD-1 pathway inhibitor in (a) and (b) is different.

53. The method of any one of claims 46-52, wherein the dose of the PD-1 pathway inhibitor in (a) and/or (b) is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

54. The method of any one of claims 1-53, wherein the CCRT comprises a platinum doublet chemotherapy (PDCT).

55. The method of claim 54, wherein the PDCT comprises a platinum agent in combination with a nucleoside analog, an antimetabolite, a taxane, a vinca alkaloid, or a topisomerase inhibitor.

56. The method of claim 55, wherein the platinum agent comprises cisplatin, carboplatin, oxaliplatin, satraplatin, picoplatin, nedaplatin, triplatin, lipoplatin, or phenanthriplatin.

57. The method of claim 55 or 56, wherein the platinum agent comprises cisplatin.

58. The method of claim 55 or 56, wherein the platinum agent comprises carboplatin.

59. The method of any one of claims 55-58, wherein the nucleoside analog comprises cytarabine, gemcitabine, lamivudine, entecavir, or telbivudine.

60. The method of claim 59, wherein the nucleoside analog comprises gemcitabine.

61. The method of any one of claims 55-58, wherein the antimetabolite comprises capecitabine, cladribine, clofarabine, cytarabine, floxuridine, fludarabine, fluorouracil, mercaptopurine, methotrexate, pemetrexed, pentostatin, pralatrexate, or thioguanine.

62. The method of claim 61, wherein the antimetabolite comprises pemetrexed.

63. The method of any one of claims 55-58, wherein the taxane comprises paclitaxel, albumin-bound paclitaxel, docetaxel, or cabazitaxel.

64. The method of any one of claims 55-58, wherein the vinca alkaloid comprises vinblastine, vincristine, vinorelbine, vindesine, vincaminol, vineridine, or vinburnine.

65. The method of claim 64, wherein the vinca alkaloid comprises vinorelbine or vinblastine.

66. The method of any one of claims 55-58, wherein the topoisomerase inhibitor comprises etoposide, mitoxantrone, doxorubicin, irinotecan, topotecan, or camptothecin.

67. The method of claim 66, wherein the topoisomerase inhibitor comprises etoposide.

68. The method of claim 66, wherein the topoisomerase inhibitor comprises irinotecan.

69. The method of any one of claims 54-69, wherein the PDCT comprises cisplatin or carboplatin in combination with gemcitabine, pemetrexed, paclitaxel, albumin-bound paclitaxel, docetaxel, vinorelbine, vinblastine, etoposide, or irinotecan.

70. The method of any one of claims 54-69, wherein the PDCT comprises cisplatin or carboplatin in combination with paclitaxel or albumin-bound paclitaxel.

71. The method of any one of claims 54-69, wherein the PDCT comprises cisplatin or carboplatin in combination with pemetrexed.

72. The method of any one of claims 54-69, wherein the PDCT comprises cisplatin or carboplatin in combination with etoposide.

73. The method of any one of claims 1-72, wherein the CCRT comprises thoracic radiotherapy and/or volumetric-modulated arc therapy (VMAT), intensity-modulated radiation therapy (IMRT), or 3-dimensional conformal radiation therapy (3DRT).

74. The method of any one of claims 1-73, wherein the LAG-3 antagonist comprises an anti-LAG-3 antibody.

75. The method of claim 74, wherein the anti-LAG-3 antibody comprises a full-length antibody.

76. The method of claim 74 or 75, wherein the anti-LAG-3 antibody comprises a monoclonal, human, humanized, chimeric, or multispecific antibody.

77. The method of claim 74, wherein the multispecific antibody comprises a DART, a DVD-Ig, or bispecific antibody.

78. The method of claim 74, wherein the anti-LAG-3 antibody comprises a F(ab′)₂ fragment, a Fab′ fragment, a Fab fragment, a Fv fragment, a scFv fragment, a dsFv fragment, a dAb fragment, or a single chain binding polypeptide.

79. The method of any one of claims 74-78, wherein the anti-LAG-3 antibody comprises BMS-986016 (relatlimab), IMP731 (H5L7BW), MK4280 (28G-10, favezelimab), REGN3767 (fianlimab), GSK2831781, humanized BAP050, IMP-701 (LAG525, ieramilimab), aLAG3 (0414), aLAG3 (0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGN02385, IBI-110, EMB-02, IBI-323, LBL-007, ABL501, or an antigen binding portion thereof.

80. The method of any one of claims 74-79, wherein the anti-LAG-3 antibody comprises CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4.

81. The method of any one of claims 74-80, wherein the anti-LAG-3 antibody comprises:
(a) a heavy chain variable region CDR1 comprising the sequence set forth in SEQ ID NO: 5;
(b) a heavy chain variable region CDR2 comprising the sequence set forth in SEQ ID NO: 6;
(c) a heavy chain variable region CDR3 comprising the sequence set forth in SEQ ID NO: 7;
(d) a light chain variable region CDR1 comprising the sequence set forth in SEQ ID NO: 8;
(e) a light chain variable region CDR2 comprising the sequence set forth in SEQ ID NO:9; and
(f) a light chain variable region CDR3 comprising the sequence set forth in SEQ ID NO:10.

82. The method of any one of claims 74-81, wherein the anti-LAG-3 antibody comprises heavy and light chain variable regions comprising the sequences set forth in SEQ ID NOs: 3 and 4, respectively.

83. The method of any one of claims 74-77 and 79-82, wherein the anti-LAG-3 antibody comprises heavy and light chains comprising the sequences set forth in SEQ ID NOs: 1 and 2, respectively.

84. The method of any one of claims 74-77 and 79-82, wherein the anti-LAG-3 antibody comprises heavy and light chains comprising the sequences set forth in SEQ ID NOs: 21 and 2, respectively.

85. The method of any one of claims 1-73, wherein the LAG-3 antagonist comprises a soluble LAG-3 polypeptide.

86. The method of claim 85, wherein the soluble LAG-3 polypeptide comprises a fusion polypeptide.

87. The method of claim 85 or 86, wherein the soluble LAG-3 polypeptide comprises a ligand binding fragment of the LAG-3 extracellular domain.

88. The method of claim 87, wherein the ligand binding fragment of the LAG-3 extracellular domain comprises an amino acid sequence with at least about 90%, at least about 95%, at least about 98%, at least about 99%, or about 100% sequence identity to SEQ ID NO:22.

89. The method of any one of claims 85-88, wherein the soluble LAG-3 polypeptide further comprises a half-life extending moiety.

90. The method of claim 89, wherein the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof.

91. The method of any one of claims 85-90, wherein the soluble LAG-3 polypeptide comprises IMP321 (eftilagimod alpha).

92. The method of any one of claims 1-91, wherein the LAG-3 antagonist is administered at a flat dose.

93. The method of any one of claims 1-92, wherein the LAG-3 antagonist is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

94. The method of any one of claims 1-93, wherein the LAG-3 antagonist is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

95. The method of any one of claims 1-91, wherein the LAG-3 antagonist is administered at a weight-based dose.

96. The method of any one of claim 1-91 or 95, wherein the LAG-3 antagonist is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

97. The method of any one of claim 1-91 or 95-96, wherein the LAG-3 antagonist is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

98. The method of any one of claims 92-97, wherein the dose of the LAG-3 antagonist is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

99. The method of any one of claims 1-98, wherein the PD-1 pathway inhibitor in (a), the CCRT, the PD-1 pathway inhibitor in (b), and/or the LAG-3 antagonist are formulated for intravenous administration.

100. The method of any one of claims 1-99, wherein the PD-1 pathway inhibitor in (b) and the LAG-3 antagonist are formulated separately.

101. The method of claim 100, wherein the PD-1 pathway inhibitor in (b) is administered before the LAG-3 antagonist.

102. The method of claim 100, wherein the LAG-3 antagonist is administered before the PD-1 pathway inhibitor in (b).

103. The method of claim 100, wherein the PD-1 pathway inhibitor in (b) and the LAG-3 antagonist are administered concurrently.

104. The method of any one of claims 1-99, wherein the PD-1 pathway inhibitor in (b) and the LAG-3 antagonist are formulated together.

105. The method of any one of claims 1-104, wherein the PD-1 pathway inhibitor in (b) and the LAG-3 antagonist are administered as a maintenance therapy.

106. The method of claim 105, wherein the maintenance therapy is administered for up to about 1 year.

107. A method of treating a human subject afflicted with a NSCLC that has a squamous or non-squamous histology, the method comprising:
(a) administering to the subject about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, and a CCRT comprising a PDCT and radiotherapy,
(b) providing the subject with a recovery period that begins upon completion of the administration in (a), followed by
(c) administering to the subject a maintenance therapy comprising about 480 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, and about 480 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO: 4.

108. The method of claim 107, wherein the PDCT comprises cisplatin and etoposide.

109. The method of claim 108, wherein (a) comprises three 21-day cycles,
wherein the anti-PD-1 antibody is administered on Day 1 of each cycle,
wherein about 100 mg/m² of the etoposide is administered on Days 1, 2, and 3 of each cycle, and after the anti-PD-1 antibody on Day 1 of each cycle, and
wherein about 80 mg/m² of the cisplatin is administered on Day 1 of each cycle after the etoposide.

110. The method of claim 108 or 109, wherein each of the cisplatin and the etoposide is administered intravenously over about 60 minutes.

111. The method of claim 107, wherein the PDCT comprises carboplatin and paclitaxel.

112. The method of claim 111, wherein (a) comprises three 21-day cycles,
wherein the anti-PD-1 antibody is administered on Day 1 of each cycle,
wherein about 175 mg/m² or about 200 mg/m² of the paclitaxel is administered on Day 1 of the first cycle, wherein about 45 mg/m² or about 50 mg/m² of the paclitaxel is administered on Days 1, 8, and 15 of the second and third cycles, and wherein the paclitaxel is administered after the anti-PD-1 antibody on Day 1 of each cycle, and
wherein the carboplatin at a target AUC of about 5 mg/mL·min or about 6 mg/mL·min is administered on Day 1 of the first cycle, wherein the carboplatin at a target AUC of about 2 mg/mL·min is administered on Days 1, 8, and 15 of the second and third cycles, and wherein the carboplatin is administered after the paclitaxel in each cycle.

113. The method of claim 111 or 112, wherein the carboplatin is administered intravenously over about 30 minutes, wherein the paclitaxel is administered intravenously over about 180 minutes in the first cycle and over about 60 minutes in the second and third cycles.

114. The method of claim 107, wherein the PDCT comprises cisplatin and pemetrexed.

115. The method of claim 114, wherein (a) comprises three 21-day cycles,
wherein the anti-PD-1 antibody is administered on Day 1 of each cycle,
wherein about 500 mg/m² of the pemetrexed is administered on Day 1 of each cycle, after the anti-PD-1 antibody, and
wherein about 75 mg/m² of the cisplatin is administered on Day 1 of each cycle after the pemetrexed.

116. The method of claim 114 or 115, wherein the cisplatin is administered intravenously over about 60 minutes and the pemetrexed is administered intravenously over about 10 minutes.

117. The method of any one of claim 108-110 or 114-116, wherein the cisplatin is replaced with carboplatin at a target area under the concentration time-curve (AUC) of about 5 mg/mL·min if the cisplatin is not tolerated by the subject.

118. The method of claim 117, wherein the carboplatin is administered intravenously over about 30 minutes.

119. The method of any one of claims 114-118, wherein the pemetrexed is replaced with etoposide if the pemetrexed is not tolerated by the subject.

120. The method of any one of claims 107-119, wherein the anti-PD-1 antibody in (a) is administered intravenously over about 30 minutes.

121. The method of any one of claims 107-120, wherein the radiotherapy is administered after the PDCT and comprises a dose of about 60 Gy to about 66 Gy.

122. The method of any one of claims 107-121, wherein the radiotherapy comprises thoracic radiotherapy and/or volumetric-modulated arc therapy (VMAT), intensity-modulated radiation therapy (IMRT), or 3-dimensional conformal radiation therapy (3DRT).

123. The method of any one of claims 107-122, wherein the radiotherapy begins on Day 1 of the second and third cycles and comprises about 30 to about 33 daily fractions of 2 Gy on a schedule of about 5 days on and 2 days off over about 6 weeks to about 7 weeks.

124. The method of any one of claims 107-123, wherein the recovery period is a duration sufficient for the subject to recover from toxicities associated with the CCRT other than fatigue, esophagitis, or alopecia.

125. The method of any one of claims 107-124, wherein the recovery period is from about 1 week to about 12 weeks, about 1 week to about 9 weeks, about 1 week to about 6 weeks, about 2 weeks to about 12 weeks, about 2 weeks to about 9 weeks, about 2 weeks to about 6 weeks, about 3 weeks to about 12 weeks, about 3 weeks to about 9 weeks, about 3 weeks to about 6 weeks, about 18 days from final administration of the anti-PD-1 antibody in (a) to about 12 weeks, about 18 days from final administration of the anti-PD-1 antibody in (a) to about 9 weeks, or about 18 days from final administration of the anti-PD-1 antibody in (a) to about 6 weeks.

126. The method of any one of claims 107-125, wherein the method is a first line therapy.

127. The method of any one of claims 107-126, wherein the subject has not received a prior local or systemic anticancer therapy given as primary therapy for locally advanced disease.

128. The method of any one of claims 107-127, wherein the subject has not received a prior systemic therapy for cancer, the subject has not received a prior systemic therapy for lung cancer, or the subject has not received a prior systemic therapy for advanced or metastatic lung cancer.

129. The method of any one of claims 107-128, wherein the subject is naïve to prior immuno-oncology therapy, the subject is naïve to prior immuno-oncology therapy for lung cancer, or the lung cancer is naïve to prior immuno-oncology therapy.

130. The method of any one of claims 107-125, wherein the method is a second line therapy.

131. The method of any one of claims 107-125, wherein the method is a third line therapy.

132. The method of claim 130 or 131, wherein the subject has progressed on a prior therapy.

133. The method of any one of claims 130-132, wherein the NSCLC is recurrent following multi-modal therapy for locally advanced NSCLC.

134. The method of any one of claims 107-133, wherein the NSCLC is unresectable, advanced, recurrent, and/or metastatic.

135. The method of any one of claims 107-134, wherein the NSCLC has a squamous or non-squamous histology.

136. The method of any one of claims 107-135, wherein the NSCLC comprises locally advanced Stage IIIA, IIIB, or IIIC NSCLC.

137. The method of any one of claims 107-136, wherein the subject is free of progressive NSCLC during the CCRT or the recovery period.

138. The method of any one of claims 107-137, wherein the anti-PD-1 and the anti-LAG-3 antibodies in (b) are administered once about every 4 weeks.

139. The method of any one of claims 107-138, wherein the anti-PD-1 and the anti-LAG-3 antibodies in (b) are formulated separately.

140. The method of claim 139, wherein the anti-PD-1 antibody in (b) is administered before the anti-LAG-3 antibody.

141. The method of claim 139, wherein the anti-LAG-3 antibody is administered before the anti-PD-1 antibody in (b).

142. The method of claim 139, wherein the anti-PD-1 and the anti-LAG-3 antibodies in (b) are administered concurrently.

143. The method of any one of claims 107-138, wherein the anti-PD-1 and the anti-LAG-3 antibodies in (b) are formulated together.

144. The method of any one of claims 107-143, wherein the anti-PD-1 and the anti-LAG-3 antibodies in (b) are administered intravenously over about 30 minutes.

145. The method of claim 107-144, wherein the anti-PD-1 antibody in (a) and/or (b) comprises a full-length antibody.

146. The method of any one of claims 107-145, wherein the anti-PD-1 antibody in (a) and/or (b) comprises a monoclonal, human, humanized, chimeric, or multispecific antibody.

147. The method of claim 146, wherein the multispecific antibody comprises a DART, a DVD-Ig, or bispecific antibody.

148. The method of claim 107-144, wherein the anti-PD-1 antibody in (a) and/or (b) comprises a F(ab′)₂ fragment, a Fab′ fragment, a Fab fragment, a Fv fragment, a scFv fragment, a dsFv fragment, a dAb fragment, or a single chain binding polypeptide.

149. The method of any one of claims 107-148, wherein the anti-PD-1 antibody in (a) and/or (b) comprises:
(a) a heavy chain variable region CDR1 comprising the sequence set forth in SEQ ID NO: 15;
(b) a heavy chain variable region CDR2 comprising the sequence set forth in SEQ ID NO: 16;
(c) a heavy chain variable region CDR3 comprising the sequence set forth in SEQ ID NO: 17;
(d) a light chain variable region CDR1 comprising the sequence set forth in SEQ ID NO: 18;
(e) a light chain variable region CDR2 comprising the sequence set forth in SEQ ID NO: 19; and
(f) a light chain variable region CDR3 comprising the sequence set forth in SEQ ID NO: 20.

150. The method of any one of claims 107-149, wherein the anti-PD-1 antibody in (a) and/or (b) comprises heavy and light chain variable regions comprising the sequences set forth in SEQ ID NOs: 13 and 14, respectively.

151. The method of any one of claims 107-147 and 149-150, wherein the anti-PD-1 antibody in (a) and/or (b) comprises heavy and light chains comprising the sequences as set forth in SEQ ID NOs: 11 and 12, respectively.

152. The method of claim 107-151, wherein the anti-LAG-3 antibody comprises a full-length antibody.

153. The method of any one of claims 107-152, wherein the anti-LAG-3 antibody comprises a monoclonal, human, humanized, chimeric, or multispecific antibody.

154. The method of claim 153, wherein the multispecific antibody comprises a DART, a DVD-Ig, or bispecific antibody.

155. The method of claim 107-151, wherein the anti-LAG-3 antibody comprises a F(ab′)₂ fragment, a Fab′ fragment, a Fab fragment, a Fv fragment, a scFv fragment, a dsFv fragment, a dAb fragment, or a single chain binding polypeptide.

156. The method of any one of claims 107-155, wherein the anti-LAG-3 antibody comprises:
(a) a heavy chain variable region CDR1 comprising the sequence set forth in SEQ ID NO: 5;
(b) a heavy chain variable region CDR2 comprising the sequence set forth in SEQ ID NO: 6;
(c) a heavy chain variable region CDR3 comprising the sequence set forth in SEQ ID NO: 7;
(d) a light chain variable region CDR1 comprising the sequence set forth in SEQ ID NO: 8;
(e) a light chain variable region CDR2 comprising the sequence set forth in SEQ ID NO: 9; and
(f) a light chain variable region CDR3 comprising the sequence set forth in SEQ ID NO: 10.

157. The method of any one of claims 107-156, wherein the anti-LAG-3 antibody comprises heavy and light chain variable regions comprising the sequences set forth in SEQ ID NOs: 3 and 4, respectively.

158. The method of any one of claims 107-154 and 156-157, wherein the anti-LAG-3 antibody comprises heavy and light chains comprising the sequences set forth in SEQ ID NOs: 1 and 2, respectively.

159. The method of any one of claims 107-154 and 156-157, wherein the anti-LAG-3 antibody comprises heavy and light chains comprising the sequences set forth in SEQ ID NOs:21 and 2, respectively.

160. The method of any one of claims 1-159, wherein one or more immune cells in tumor tissue from the subject express LAG-3.

161. The method of claim 160, wherein at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3.

162. The method of claim 160 or 161, wherein at least about 1% of the immune cells express LAG-3.

163. The method of any one of claims 160-162, wherein the immune cells comprise tumor-infiltrating lymphocytes.

164. The method of claim 163, wherein the tumor-infiltrating lymphocytes comprise CD8⁺ cells.

165. The method of any one of claims 1-159, wherein one or more nucleated cells in tumor tissue from the subject express LAG-3.

166. The method of claim 165, wherein at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the nucleated cells express LAG-3.

167. The method of claim 165 or 166, wherein at least about 1% of the nucleated cells express LAG-3.

168. The method of any one of claims 1-167, wherein one or more tumor cells in tumor tissue from the subject express PD-L1.

169. The method of claim 168, wherein at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1.

170. The method of claim 168 or 169, wherein at least about 1% of the tumor cells express PD-L1.

171. The method of any one of claims 1-170, further comprising administering to the subject an additional therapeutic agent.

172. The method of claim 171, wherein the additional therapeutic agent comprises an anti-cancer agent.

173. The method of claim 172, wherein the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

174. The method of claim 173, wherein the tyrosine kinase inhibitor comprises afatinib, erlotinib, dacomitinib, gefitinib, osimertinib, alectinib, brigatinib, ceritinib, crizotinib, lorlatinib, entrectinib, dabrafenib, trametinib, vemurafenib, larotrectinib, or any combination thereof.

175. The method of claim 173, wherein the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGF receptor (EGFR), or any combination thereof.

176. The method of claim 173 or 175, wherein the anti-angiogenesis agent comprises bevacizumab, ramucirumab, aflibercept, tanibirumab, olaratumab, nesvacumab, AMG780, MEDI3617, vanucizumab, rilotumumab, ficlatuzumab, TAK-701, onartuzumab, emibetuzumab, or any combination thereof.

177. The method of claim 173, wherein the checkpoint inhibitor comprises a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

178. The method of any one of claim 173 or 177, wherein the checkpoint inhibitor comprises a CTLA-4 inhibitor.

179. The method of claim 178, wherein the CTLA-4 inhibitor comprises an anti-CTLA-4 antibody.

180. The method of claim 179, wherein the anti-CTLA-4 antibody comprises a full-length antibody.

181. The method of any one of claim 179 or 180, wherein the anti-CTLA-4 antibody comprises a monoclonal, human, humanized, chimeric, or multispecific antibody.

182. The method of claim 181, wherein the multispecific antibody comprises a DART, a DVD-Ig, or bispecific antibody.

183. The method of claim 179, wherein the anti-CTLA-4 antibody comprises a F(ab′)₂ fragment, a Fab′ fragment, a Fab fragment, a Fv fragment, a scFv fragment, a dsFv fragment, a dAb fragment, or a single chain binding polypeptide.

184. The method of any one of claims 179-183, wherein the anti-CTLA-4 antibody comprises ipilimumab, tremelimumab, MK-1308, AGEN-1884, or comprises an antigen binding portion thereof.
